SEQUENCE LISTING (92 sequences)

SEQ ID NO: 1 - protein, 13 aa, synthetic construct
XXPRXSHKGPXPF

SEQ ID NO: 2 - protein, 13 aa, synthetic construct
XXPRLSXKGPXPF

SEQ ID NO: 3 - protein, 13 aa, synthetic construct
XXPAXSHKGPXPF

SEQ ID NO: 4 - protein, 13 aa, synthetic construct
XXPALXHKGPXPF

SEQ ID NO: 5 - protein, 13 aa, synthetic construct
XXPALSXKGPXPF

SEQ ID NO: 6 - protein, 13 aa, synthetic construct
XXPALSHXGPXPF

SEQ ID NO: 7 - protein, 13 aa, synthetic construct
XRPRLSHKXPXPX

SEQ ID NO: 8 - protein, 13 aa, synthetic construct
XRPRLSHKXPXPX

SEQ ID NO: 9 - protein, 13 aa, synthetic construct
XRPRLSHKGPXPX

SEQ ID NO: 10 - protein, 13 aa, synthetic construct
XRPRLSHKGPXPX

SEQ ID NO: 11 - protein, 12 aa, synthetic construct
XRXRLSXKGXPF

SEQ ID NO: 12 - protein, 13 aa, synthetic construct
XRXRLSDKGPXPF

SEQ ID NO: 13 - protein, 13 aa, synthetic construct
XRXRLSXKGPXPF

SEQ ID NO: 14 - protein, 13 aa, synthetic construct
XRXRLSXKGPXPF

SEQ ID NO: 15 - protein, 13 aa, synthetic construct
XRKRLSDKGPXPF

SEQ ID NO: 16 - protein, 13 aa, synthetic construct
XRXRLSXKGPXPF

SEQ ID NO: 17 - protein, 13 aa, unidentified
XRXRLSXKGPXPF

SEQ ID NO: 18 - protein, 11 aa, synthetic construct
XRLSXKGPXPF

SEQ ID NO: 19 - protein, 11 aa, synthetic construct
XRLSXKGPXPF

SEQ ID NO: 20 - protein, 11 aa, synthetic construct
XRLSXKGPXPF

SEQ ID NO: 21 - protein, 11 aa, synthetic construct
XRLSXKGPXPF

SEQ ID NO: 22 - protein, 11 aa, synthetic construct
XXLSXKGPXPF

SEQ ID NO: 23 - protein, 11 aa, synthetic construct
XRLSXKGPXPF

SEQ ID NO: 24 - protein, 11 aa, synthetic construct
XRLSXKGPXPF

SEQ ID NO: 25 - protein, 11 aa, synthetic construct
XRLSXKGPXPF

SEQ ID NO: 26 - protein, 11 aa, synthetic construct
XRLSXKGPXPF

SEQ ID NO: 27 - protein, 9 aa, synthetic construct
XRLSXKGPX

SEQ ID NO: 28 - protein, 6 aa, synthetic construct
XRLSXK

SEQ ID NO: 29 - protein, 8 aa, synthetic construct
XRLSXGPX

SEQ ID NO: 30 - protein, 8 aa, synthetic construct
XRLSXXPX

SEQ ID NO: 31 - protein, 7 aa, synthetic construct
XRLSXPX

SEQ ID NO: 32 - protein, 9 aa, synthetic construct
XRLSXKGPA

SEQ ID NO: 33 - protein, 9 aa, synthetic construct
XRLSXKGPX

SEQ ID NO: 34 - protein, 9 aa, synthetic construct
XRLSXKGPX

SEQ ID NO: 35 - protein, 9 aa, synthetic construct
XRLSXKGPX

SEQ ID NO: 36 - protein, 9 aa, synthetic construct
XRLSXKGPX

SEQ ID NO: 37 - protein, 9 aa, synthetic construct
XRLSXKGPX

SEQ ID NO: 38 - protein, 9 aa, synthetic construct
XRLSXKGPX

SEQ ID NO: 39 - protein, 9 aa, synthetic construct
XRLSXKGPX

SEQ ID NO: 40 - protein, 9 aa, synthetic construct
XRLSXKGPX

SEQ ID NO: 41 - protein, 9 aa, synthetic construct
XRLSXKGPX

SEQ ID NO: 42 - protein, 9 aa, synthetic construct
XRLSXKGPX

SEQ ID NO: 43 - protein, 9 aa, synthetic construct
XRLSXKGPY

SEQ ID NO: 44 - protein, 17 aa, synthetic construct
ENTNXRPRLKHKGPXPF

SEQ ID NO: 45 - protein, 17 aa, synthetic construct
DTDWXRPRLSHXGPXPF

SEQ ID NO: 46 - protein, 5 aa, synthetic construct
RKPEX

SEQ ID NO: 47 - protein, 13 aa, Homo sapiens
XRPRLSHKGPMPF

SEQ ID NO: 48 - protein, 17 aa, synthetic construct
XFRRQRPRLSHKGPMXF

SEQ ID NO: 49 - protein, 17 aa, synthetic construct
XFRRQRPRLEHAKPAPF

SEQ ID NO: 50 - protein, 17 aa, synthetic construct
XFRRQRPRLEHKKPXPX

SEQ ID NO: 51 - protein, 17 aa, synthetic construct
XFRRQRPRLEHKKPXPX

SEQ ID NO: 52 - protein, 17 aa, synthetic construct
XFRRQRPRLEHKKPXPX

SEQ ID NO: 53 - protein, 17 aa, synthetic construct
XFRRQRPRLEHKKPXPX

SEQ ID NO: 54 - protein, 17 aa, synthetic construct
XFRRQRPRLEHKKPXPX

SEQ ID NO: 55 - protein, 17 aa, synthetic construct
XFRRQRPRLEHKKPXPX

SEQ ID NO: 56 - protein, 17 aa, synthetic construct
XFRRQRPRLEHKKPXPX

SEQ ID NO: 57 - protein, 17 aa, Synthetic construct
XFRRQRPRLEHKKPXPX

SEQ ID NO: 58 - protein, 14 aa, synthetic construct
XRRCXPLHSRVPFP

SEQ ID NO: 59 - protein, 14 aa, synthetic construct
KRREXPLHSRVPFP

SEQ ID NO: 60 - protein, 14 aa, synthetic construct
KRREXPLHSRVXFP

SEQ ID NO: 61 - protein, 14 aa, synthetic construct
XRRKXPEHSRVPFP

SEQ ID NO: 62 - protein, 14 aa, synthetic construct
XRREXPKHSRVPFP

SEQ ID NO: 63 - protein, 14 aa, synthetic construct
XRRSKPLHERVPFP

SEQ ID NO: 64 - protein, 14 aa, synthetic construct
XRRSEPLHKRVPFP

SEQ ID NO: 65 - protein, 14 aa, synthetic construct
XRRSEPLHKRVXFP

SEQ ID NO: 66 - protein, 14 aa, synthetic construct
KRREXCLHCRVPFP

SEQ ID NO: 67 - protein, 11 aa, synthetic construct
KXPLESRVPFP

SEQ ID NO: 68 - protein, 11 aa, synthetic construct
EXPLKSRVPFP

SEQ ID NO: 69 - protein, 10 aa, synthetic construct
XELHKRVPFP

SEQ ID NO: 70 - protein, 10 aa, synthetic construct
XPKHSREPFP

SEQ ID NO: 71 - protein, 10 aa, synthetic construct
XPEHSRKPFP

SEQ ID NO: 72 - protein, 12 aa, synthetic construct
XPLEHSRKVPFP

SEQ ID NO: 73 - protein, 11 aa, Synthetic construct
XPLEHRKVPFP

SEQ ID NO: 74 - protein, 10 aa, synthetic construct
XPLHKRVEFP

SEQ ID NO: 75 - protein, 10 aa, synthetic construct
XPLHERVKFP

SEQ ID NO: 76 - protein, 14 aa, synthetic construct
XRRSEPLHKRVPFP

SEQ ID NO: 77 - protein, 14 aa, synthetic construct
XXRSEPLHKRVPFP

SEQ ID NO: 78 - protein, 14 aa, synthetic construct
XRXSEPLHKRVPFP

SEQ ID NO: 79 - protein, 14 aa, synthetic construct
XRRSEPLHKRVPXP

SEQ ID NO: 80 - protein, 14 aa, synthetic construct
XRRSEPLHKRVPFX

SEQ ID NO: 81 - protein, 14 aa, synthetic construct
XRRSEPLHKRVPXP

SEQ ID NO: 82 - protein, 13 aa, synthetic construct
XRPRLSHKGPXPF

SEQ ID NO: 83 - protein, 32 aa, Homo sapiens
XRPVNLTMRRKLRKHNCLQRRCMPLHSRVPFP

SEQ ID NO: 84 - protein, 13 aa, Synthetic construct
XRXRLSXKGPXPF

SEQ ID NO: 85 - protein, 14 aa, Homo sapiens
XRRCMPLHSRVPFP

SEQ ID NO: 86 - protein, 17 aa, Homo sapiens
KFRRQRPRLSHKGPMPF

SEQ ID NO: 87 - protein, 12 aa, synthetic construct
XXXLXXXGPXPF

SEQ ID NO: 88 - protein, 12 aa, synthetic construct
XXXLXXXGPXPF

SEQ ID NO: 89 - protein, 13 aa, synthetic construct
XXXXLXXXGPXPF

SEQ ID NO: 90 - protein, 12 aa, synthetic construct
XXXLXXXGPXPF

SEQ ID NO: 91 - protein, 12 aa, synthetic construct
XXXLXXXGPXPF

SEQ ID NO: 92 - protein, 13 aa, Synthetic construct
XXXXLXXXGPXPF

(19) United States
(12) Patent Application Publication
(10) Pub. No.: US 2024/0343761 A1
(43) Pub. Date: Oct. 17, 2024

(54) APELINERGIC MACROCYCLES AND USES THEREOF

(71) Applicant: SOCPRA SCIENCES SANTÉ ET HUMAINES S.E.C., Sherbrooke (CA)

(72) Inventors: Philippe SARRET, Sherbrooke (CA); Pierre-Luc BOUDREAULT, Sherbrooke (CA); Alexandre MURZA, Sherbrooke (CA); Kien TRAN, Krong Kmar (VN); Éric MARSAULT, Sherbrooke (CA); Jean-Michel LONGPRÉ, Magog (CA); Jérôme CÔTÉ, Sherbrooke (CA)

(21) Appl. No.: 18/744,292

(22) Filed: Jun. 14, 2024

Related U.S. Application Data
(63) Continuation of application No. PCT/CA2022/051838, filed on Dec. 15, 2022.
(60) Provisional application No. 63/290,394, filed on Dec. 16, 2021.

(51) Int. Cl. C07K 7/56; A61K 38/00; A61P 9/02

(52) U.S. Cl. CPC C07K 7/56; A61P 9/02; A61K 38/00

(57) ABSTRACT

Aperlinergic macrocyclic compounds are provided. In particular, a compound of formula (II):

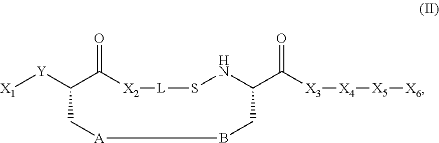

or a stereoisomer or a mixture thereof, or a pharmaceutically acceptable salt, ester or solvate thereof is provided. Also provided is a method of using aperlinergic macrocyclic compounds of the disclosure for treating a cardiovascular disease in a subject in need thereof, comprising administering an effective amount of the compound to the subject.

### CROSS REFERENCE TO RELATED APPLICATIONS

This application is a Continuation of PCT Application No. PCT/CA2022/051838 filed on Dec. 15, 2022, which claims benefit of U.S. Provisional Application No. 63/290,394, filed on Dec. 16, 2021. All documents above are incorporated herein in their entirety by reference.

### FIELD OF THE DISCLOSURE

The present disclosure relates to apelinergic macrocycles and uses thereof. More specifically, the present disclosure is concerned with apelinergic macrocycles derived from apelin-13, apelin-17 and Elabela.

### INCORPORATION BY REFERENCE OF SEQUENCE LISTING

The instant application contains a Sequence Listing which has been submitted electronically in XML format and is hereby incorporated by reference in its entirety. Said XML copy, created on Jun. 12, 2024, is named SVI-012US1_SL.xml and is 83,983 bytes in size.

### BACKGROUND OF THE DISCLOSURE

The APJ receptor is a regulator of the cardiovascular system and of metabolic activities. The two endogenous ligands, apelin and ELABELA, bind the APJ receptor with affinity in nanomolar order and they stimulate cardiac contraction, reducing resistance peripheral vascular disease, proliferation of endothelial cells and the formation of new blood vessels. Their protective effects have been demonstrated in pathological models of myocardial ischemia, cerebral ischemia, diabetes, pulmonary arterial hypertension, sepsis, and neuropathic pain. However, these endogenous peptides have poor plasma stability which limits their use. A majority of studies show that apelin and ELABELA must be administered by continuous infusion to maintain their therapeutic efficacy.

Adrenergic drugs analogues are used as standard treatments in heart dysfunction associated with sepsis. They are not always effective however and cause multiple side effects such as myocardial or peripheral ischemia. Resistance to treatment is a recurrent problem for pulmonary arterial hypertension. The available drugs have shown variable effectiveness and prognosis for this disease remains poor. Pain relievers other than opioids are also needed to avoid dose escalation and side effects.

There is need for improved apelinergic analogs.

The present description refers to a number of documents, the content of which is herein incorporated by reference in their entirety.

### SUMMARY OF THE DISCLOSURE

The present disclosure provides novel macrocyclic analogues of apelin 13, apelin 17 and Elabela (apelinergic macrocycles). Macrocyclization has been applied to apelin isoforms and Elabela, resulting in molecules with significantly improved stability (plasma half-life between 5 hours and 24 hours as compared to those of apelin-13 and Elabela 24-30 min). Affinity was measured by the displacement of the radiolabeled ligand (Nle75, Tyr77) [125I]]-Pyr-Apelin13, which indicates that some Macrocyclic peptides exhibit an affinity on APJ similar to apelin (Ki 0.2-5.7 nM).

In specific embodiments, macrocyclic analogues of the disclosure have reduced sizes (33% reduction in mass molecular vs. apelin-13), while keeping a good affinity with the receptor (Ki 0.8-5 nM vs apelin-13, Ki 0.8 nM).

In specific embodiments, macrocyclic apelinergic analogs produce cardiovascular effects comparable to endogenous ligands.

More specifically, in accordance with the present disclosure, there are provided the following items and items':

Item 1. A compound of any one of formula (I) to (VIII), or a stereoisomer or a mixture thereof, or a pharmaceutically acceptable salt, ester or solvate thereof.

Item 2. The compound of item 1, which is any one of compounds 3-4, 9-29, 35-46, 62-70, 72-79, 84, and 89-94 of Tables I to III.

Item 3. A pharmaceutical composition comprising the compound, stereoisomer, mixture, pharmaceutically acceptable salt, ester or solvate of item 1 or 2, and at least one pharmaceutically acceptable carrier or excipient.

Item 4. A method of using a compound of any one of formula (I) to (IV) for treating a cardiovascular disease in a subject in need thereof, comprising administering an effective amount of the compound to the subject.

Item 5. The method of item 4, wherein the compound is any one of compounds 3-4, 9-29, and 35-46.

Item 6. The method of item 5, wherein the compound is compound 42 or 43.

Item′ 1. A compound of formula (II):
(II)
wherein:
X1 is absent, or is X7-X8, wherein X7 is —(CH2)q-CH3 or —(CF2)q-CF3 wherein q is 0 to 11, a natural amino acid, a synthetic amino acid, the side chain of which is H, a —(C1-C12)alkyl, —(CF2)q-CF3 wherein q is 0 to 11, —(C3-C8)heteroalkyl, a —(CH2)p-(C3-C8)aryl, —(CH2)p-(C3-C8)heteroaryl, —(CH2)p-(C3-C8)cycloalkyl, or —(CH2)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S, —(C1-C6)alkyl, —O—(C1-C6)alkyl, —(CH2)p-(C3-C8)aryl, —O—(CH2)p-(C3-C8)aryl, —(C3-C8)cycloalkyl, or —O—(C3-C8)cycloalkyl, and wherein the heteroatom in the heteroalkyl, heteroaryl or heterocycloalkyl is a N, O or S; and
X8 is absent, or is a natural or synthetic amino acid, the side chain of which is —CH2—(CH2)p-NH2, —CH2—(CH2)p-guanidine, —(CH2)p-(C3-C8)cycloalkyl, —(CH2)p-(C3-C8)heterocycloalkyl, —(CH2)p-(C3-C8)aryl, or —(CH2)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S;
Y is absent, NH2-, Ac—NH—, guanidine, or H;
A is —(CH2)n-; —(CH2)nNH═C(NH2)N-CH2-CH═CH— (preferably allyl-glycine or Nα-allyl-arginine), wherein n is 2, 3 or 4; or —CH═CH—(CH2)m-, wherein m is 0, 1 or 2;
B is absent or
wherein R is O, P, m-alkyl, halogen or nitro and n is 1, 2, or 3; wherein R is H, C3-C7 alkyl, benzyl or arylalkyle and n is 1, 2 or 3; wherein n is 1, 2, 3 or 4 and m is 0 or 1; or wherein X9 is CH or N;
X2 and X3 are each independently absent, or a natural or synthetic amino acid, the side chain of which is —CH2—(CH2)p-NH2, CH2—(CH2)p-guanidine, —(CH2)p-(C3-C8)cycloalkyl, —(CH2)p-(C3-C8)heterocycloalkyl, —(CH2)p-(C3-C8)aryl, or —(CH2)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S;
X4 is a natural or non-natural amino acid having a positively charged or uncharged sidechain;
X5 is Gly, Phe, Leu, Ile, Ser, Aib, Pro, Sar, Oic, βAla, Hyp or Hyp(OBn); and
X6 is X10-X11-X12, wherein
X10 is any natural amino acid; or a synthetic amino acid, the side chain of which is H, —(CH2)p-(C3-C8)alkyl, —(CH2)p-(C3-C8)heteroalkyl, —(CH2)p-(C3-C8)cycloalkyl, —(CH2)p-(C3-C8)heterocycloalkyl, —(CH2)p-(C3-C8)aryl, —(CH2)p-(C3-C8)heteroaryl, —CH2—(CH2)p-NH2, —CH2—(CH2)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S, —(C1-C6)alkyl, —O—(C1-C6)alkyl, —(CH2)p′—(C3-C8)aryl, —O—(CH2)p′—(C3-C8)aryl, —(C3-C8)cycloalkyl, or —O—(C3-C8)cycloalkyl, wherein p′ is 0 to 5; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment, X10 is an amino acid, the side chain of which is —(CH2)p-(C3-C8)alkyl, or —(CH2)p-(C3-C8)aryl, wherein p is 0 to 5, wherein the aryl is optionally fused with one or two (C3-C8)aryl, and wherein the aryl is optionally substituted with one or more substituents, wherein each substituent is independently 0—(C1-C6)alkyl, —(CH2)p-(C3-C8)aryl, —O—(CH2)p-(C3-C8)aryl, —(C3-C8)cycloalkyl, or —O—(C3-C8)cycloalkyl, wherein p is 0 to 5. In a specific embodiment, it is not Ala. In a more specific embodiment, X10 is Nle, alpha-methylleucine, cycloleucine, tert-leucine, cyclohexylalanine (e.g., (3-cyclohexyl-L-alanine), alpha-methylphenylalanine, Phe, Tic ((S)—N-Fmoc-tetrahydroisoquinoline-3-carboxylic acid), Tyr, 1Nal, 2Nal, TyrOBn, cypTyr(OBn), dcypTyr(OBn), cypTyr(OCyp), cypTyr(OPr), D-1Nal, D-2Nal, D-TyrOBn, or D-Tyr;
X11 is absent or Gly, Phe, Leu, Ile, Ser, Aib, Pro, Sar, Oic, βAla, Hyp or Hyp(OBn). In a specific embodiment, it is absent or Pro; and
X12 is absent or Phe,
or a stereoisomer or a mixture thereof, or a pharmaceutically acceptable salt, ester or solvate thereof.

Item′2. The compound of item′ 1, wherein:
X2 and X3 are each independently an amino acid, the side chain of which is —CH2-(CH2)p-guanidine, —CH2—(CH2)p-NH2, or —(CH2)p-imidazole, preferably —CH2—(CH2)p-guanidine, or —CH2—(CH2)p-NH2, wherein p is 0 to 4; and/or
X10 is an amino acid, the side chain of which is —(CH2)p-(C3-C8)alkyl, or —(CH2)p-(C3-C8)aryl, wherein p is 0 to 5, wherein the aryl is optionally fused with one or two (C3-C8)aryl, and wherein the aryl is optionally substituted with one or more substituents, wherein each substituent is independently —OH, —O—(C1-C6)alkyl, —(CH2)p′—(C3-C8)aryl, —O—(CH2)p′—(C3-C8)aryl, —(C3-C8)cycloalkyl, or —O—(C3-C8)cycloalkyl, wherein p′ is 0 to 5, or a stereoisomer or a mixture thereof, or a pharmaceutically acceptable salt, ester or solvate thereof.

Item′3. The compound of item′ 1, wherein:
X2 and X3 are each independently Lys, Orn, Dab (2,4-diaminobutyric acid), Dap (2,3-diaminopropionic acid), Arg, hArg, His, Nle, alpha-methylleucine, cycloleucine, tert-leucine, cyclohexylalanine (e.g., (3-cyclohexyl-L-alanine) or alpha-methylphenylalanine;
X4 is Gly, Phe, Leu, Ile, Ser, Aib, Pro, Sar, Oic, βAla, Hyp or Hyp(OBn);
X5 is Gly, Phe, Leu, Ile, Ser, Aib, Pro, Sar, Oic, βAla, Hyp or Hyp(OBn); and/or
X10 is X10 is Nle, alpha-methylleucine, cycloleucine, tert-leucine, cyclohexylalanine (e.g., (3-cyclohexyl-L-alanine), alpha-methylphenylalanine, Phe, Tic ((S)—N-Fmoc-tetrahydroisoquinoline-3-carboxylic acid), Tyr, 1Nal, 2Nal, TyrOBn, cypTyr(OBn), dcypTyr(OBn), cypTyr(OCyp), cypTyr(OPr), D-1Nal, D-2Nal, D-TyrOBn, or D-Tyr, or a stereoisomer or a mixture thereof, or a pharmaceutically acceptable salt, ester or solvate thereof.

Item′4. The compound of item′ 3, wherein:
X2 and X3 are each independently Lys, Arg, hArg, Nle, Leu, Phe, or Cha;
X4 is Gly; and/or
X5 is Pro, or a stereoisomer or a mixture thereof, or a pharmaceutically acceptable salt, ester or solvate thereof.

Item′5. The compound of any one of item's 1 to 4, wherein:
X1 is absent;
Y is NH2, Ac—NH—, guanidine, or H;
A is —CH═CH—(CH2)m-, wherein m is 0, 1 or 2;
B is absent, wherein R is O, P, m-alkyl, halogen or nitro and n is 1, 2, or 3, , wherein X9 is CH or N; and/or
X10 is an amino acid, the side chain of which is —(CH2)p-(C3-C8)alkyl, or —(CH2)p-(C3-C8)aryl, wherein p is 0 to 5, wherein the aryl is optionally fused with one or two (C3-C8)aryl, and wherein the aryl is optionally substituted with one or more substituents, wherein each substituent is independently —OH, —O—(C1-C6)alkyl, —(CH2)p′—(C3-C8)aryl, —O—(CH2)p′—(C3-C8)aryl, —(C3-C8)cycloalkyl, or —O—(C3-C8)cycloalkyl, wherein p′ is 0 to 5,
   or a stereoisomer or a mixture thereof, or a pharmaceutically acceptable salt, ester or solvate thereof.

Item′6. The compound of item′ 5, wherein X10 is Ne or D-1Nal, or a stereoisomer or a mixture thereof, or a pharmaceutically acceptable salt, ester or solvate thereof.

2. The compound of any one of item's 1 to 4, wherein:
X1 is X7-X8; and/or
Y is absent,
   or a stereoisomer or a mixture thereof, or a pharmaceutically acceptable salt, ester or solvate thereof.

Item′7. The compound of item′ 7, wherein:
X1 is X7-X8 and X8 is an amino acid, the side chain of which is —CH2—(CH2)p-guanidine, —CH2—(CH2)p-NH2, or —(CH2)p-imidazole, preferably —CH2—(CH2)p-guanidine, or —CH2—(CH2)p-NH2, wherein p is 0 to 4,
   or a stereoisomer or a mixture thereof, or a pharmaceutically acceptable salt, ester or solvate thereof.

Item′8. The compound of item′ 7 or 8, wherein
A is —(CH2)n- or —CH═CH—(CH2)m-, wherein m is 0, 1 or 2,
   or a stereoisomer or a mixture thereof, or a pharmaceutically acceptable salt, ester or solvate thereof.

Item′9. A compound of any one of formula (I) to (VIII), or a stereoisomer or a mixture thereof, or a pharmaceutically acceptable salt, ester or solvate thereof.

Item′10. The compound of item′ 9, which is any one of compounds 3-4, 9-29, 35-46, 62-70, 72-79, 84, and 89-94, preferably any one of compounds 11, 13, 15-16, 18-20, and 42-44:

| Compound | | |
|---|---|---|
| No. | Name | Structure |
| 3 | KT01-16 | Pyr-c[X-P-R-X]c-S-H-K-G-P-Nle-P-F |
| 4 | KT01-17 | Pyr-c[X-P-R-L-S-X]c-K-G-P-Nle-P-F |
| 9 | KT02-98 | Pyr-R-P-R-L-S-H-K-[dX-P-Nle-P-X] |
| 10 | KT03-32 | Pyr-R-P-R-L-S-H-K-[X-P-Nle-P-dX] |
| 11 | KT02-136 | Pyr-R-P-R-L-S-H-K-G-P-[X-P-X] |
| 12 | KT02-137 | Pyr-R-P-R-L-S-H-K-G-P-[dX-P-X] |
| 13 | KT01-125 | Pyr-R-c*[X-R-L-S-X]c-K-G-P-Nle-P-F |
| 14 | KT01-105 | Pyr-R-c[Dap-R-L-S-Asp]c-K-G-P-Nle-P-F |
| 15 | KT01-98 | Pyr-R-c[X-R-L-S-Alh]c-K-G-P-Nle-P-F |
| 16 | KT01-123 | Pyr-R-c[X-R-L-S-Alnb]c-K-G-P-Nle-P-F |
| 17 | KT01-106 | Pyr-R-c[Lys-R-L-S-Asp]c-K-G-P-Nle-P-F |
| 18 | KT01-126 | Pyr-R-c[X-R-L-S-Alb]c-K-G-P-Nle-P-F |
| 19 | KT01-122 | Pyr-R-c[X-R-L-S-Almb]c-K-G-P-Nle-P-F |
| 20 | KT01-100 | NH2-c[X-R-L-S-X]c-K-G-P-Nle-P-F |
| 21 | KT01-118 | Ac-NH-c[X-R-L-S-X]c-K-G-P-Nle-P-F |
| 22 | KT01-110 | Ø-c[X-R-L-S-X]c-K-G-P-Nle-P-F |
| 23 | KT01-121 | Guanidine-c[X-R-L-S-X]c-K-G-P-Nle-P-F |
| 24 | KT01-133 | NH2-c[X-Nle-L-S-X]c-K-G-P-Nle-P-F |
| 25 | KT01-127 | NH2-c[X-R-L-S-Alh]c-K-G-P-Nle-P-F |
| 26 | KT01-120 | NH-c[Rx-R-L-S-AlH]-K-G-P-Nle-P-F |
| 27 | KT01-111 | Ø-c[X-R-L-S-AlH]-K-G-P-Nle-P-F |
| 28 | KT01-135 | NH2-c[X-R-L-S-Alnb]c-K-G-P-Nle-P-F |
| 29 | KT01-116 | NH2-c[X-R-L-S-X]c-K-G-P-Nle |
| 35 | KT03-57 | NH2-c[X-R-L-S-X]c-K-G-P-1Nal |
| 36 | KT03-58 | NH2-c[X-R-L-S-X]c-K-G-P-2Nal |
| 37 | KT03-51 | NH2-c[X-R-L-S-X]c-K-G-P-TyrOBn |
| 38 | KT03-67 | NH2-c[X-R-L-S-X]c-K-G-P-cypTyr(OBn) |
| 39 | KT03-68 | NH2-c[X-R-L-S-X]c-K-G-P-dcypTyr(OBn) |
| 40 | KT03-69 | NH2-c[X-R-L-S-X]c-K-G-P-cypTyr(OCyp) |
| 41 | KT03-70 | NH2-c[X-R-L-S-X]c-K-G-P-cypTyr(OPr) |
| 42 | KT04-43 | NH2-c[X-R-L-S-X]c-K-G-P-(D-1Nal) |
| 43 | KT04-44 | NH2-c[X-R-L-S-X]c-K-G-P-(D-2Nal) |
| 44 | KT04-42F1 | NH2-c[X-R-L-S-X]c-K-G-P-(D-TyrOBn) |
| 45 | KT04-42b | NH2-c[X-R-L-S-X]c-K-G-P-(D-Tyr) |
| 46 | KT01-145 | Ac-c[E-N-T-N-(8-aminooctanoic)-R-P-R-L-K]-H-K-G-P-Nle-P-F |
| 62 | KT02-62 | Ac-K-F-R-R-Q-R-P-R-L-[E-H-A-K]-P-A-P-F |
| 63 | KT02-76 | Ac-K-F-R-R-Q-R-P-R-L-[E-H-K-K]-P-Nle-P-cypTyrOBn |
| 64 | KT02-78 | Ac-K-F-R-R-Q-R-P-R-L-[E-H-K-K]-P-Nle-P-cypY |
| 65 | KT02-99 | Ac-K-F-R-R-Q-R-P-R-L-[E-H-K-K]-P-Nle-P-dcypTyrOBn |
| 66 | KT03-02 | Ac-K-F-R-R-Q-R-P-R-L-[E-H-K-K]-P-Nle-P-TyrOBn |
| 67 | KT02-18 | Ac-K-F-R-R-Q-R-P-R-L-[E-H-K-K]-P-Nle-P-B1 |
| 68 | KT02-19 | Ac-K-F-R-R-Q-R-P-R-L-[E-H-K-K]-P-Nle-P-B2 |
| 69 | KT02-20 | Ac-K-F-R-R-Q-R-P-R-L-[E-H-K-K]-P-Nle-P-B3 |
| 70 | KT02-21 | Ac-K-F-R-R-Q-R-P-R-L-[E-H-K-K]-P-Nle-P-B4 |
| 72 | AM03-37 | c[K-R-R-E]-Nle-P-L-H-S-R-V-P-F-P |
| 73 | AM03-66 | c[K-R-R-E]-Nle-P-L-H-S-R-V-Oic-F-P |
| 74 | AM03-38 | Pyr-R-R-c[K-Nle-P-E]-H-S-R-V-P-F-P |
| 75 | ABB01-105 | Pyr-R-R-c[E-Nle-P-K]-H-S-R-V-P-F-P |
| 76 | AM03-40 | Pyr-R-R-S-c[K-P-L-H-E]-R-V-P-F-P |
| 77 | ABB01-106 | Pyr-R-R-S-c[E-P-L-H-K]-R-V-P-F-P |
| 78 | AM03-67 | Pyr-R-R-S-c[E-P-L-H-K]-R-V-Oic-F-P |
| 79 | AM03-68 | c[K-R-R-E]-Nle-c[C-L-H-C]-R-V-P-F-P |
| 84 | ABB01-109 | Nle-P-c[E-H-S-R-K]-P-F-P |
| 89 | KT03-14 | 4BrBz-R-R-S-[E-P-L-H-K]-R-V-P-F-P |
| 90 | KT03-16 | Pyr-hR-R-S-[E-P-L-H-K]-R-V-P-F-P |
| 91 | KT03-17 | Pyr-R-hR-S-[E-P-L-H-K]-R-V-P-F-P |
| 92 | KT03-15 | Pyr-R-R-S-[E-P-L-H-K]-R-V-P-4BrF-P |
| 93 | KT03-19 | Pyr-R-R-S-[E-P-L-H-K]-R-V-P-F-Hyp(OBn) |
| 94 | KT04-16 | 4BrBz-R-R-S-[E-P-L-H-K]-R-V-P-4BrF-P |

or a stereoisomer or a mixture thereof, or a pharmaceutically acceptable salt, ester or solvate thereof.

Item′11. The compound of item′ 10, which is any one of compounds 13-25, 27-29, 35-37 and 42-45, or a stereoisomer or a mixture thereof, or a pharmaceutically acceptable salt, ester or solvate thereof.

Item′12. A pharmaceutical composition comprising the compound, stereoisomer, mixture, pharmaceutically acceptable salt, ester or solvate of any one of item's 1 to 11, and at least one pharmaceutically acceptable carrier or excipient.

Item′13. A method of using a compound of any one of formula (I) to (IV), or a stereoisomer or a mixture thereof, or a pharmaceutically acceptable salt, ester or solvate thereof, for treating a cardiovascular disease in a subject in need thereof, comprising administering an effective amount of the compound to the subject.

Item′14. The method of item′ 13, wherein the compound is any one of compounds 3-4, 9-29, and 35-46 as defined in item′ 10, or a stereoisomer or a mixture thereof, or a pharmaceutically acceptable salt, ester or solvate thereof.

Item′15. The method of item′ 13, wherein the compound is of formula (II) as defined in any one of item's 1 to 8.

Item′16. The method of item′ 15, wherein the compound is any one of compounds 13-25, 27-29, 35, 36-37 and 42-45, preferably any one of compounds 13, 15-16, 18-20, 23 and 42-44, as defined in item′ 10, or a stereoisomer or a mixture thereof, or a pharmaceutically acceptable salt, ester or solvate thereof.

Item′17. The method of item′ 16, wherein the compound is compound 42 or 43, or a stereoisomer or a mixture thereof, or a pharmaceutically acceptable salt, ester or solvate thereof.

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1: Structure of Ape13 and compound 97. The cyclisation positions, Pro3 and His7, were indicated and encircled.

FIGS. 2A-2B: Macrocyclic Ape13 analogs with various linkers (FIG. 2A) and various non-natural residues (FIG. 2B).

FIGS. 3A-3B: Macrocyclization by ring closing metathesis to produce precursors of 97, 16, 18, and 19. Linear and cyclic precursors of 97 (SEQ ID NOs: 87-89) (FIG. 3A) and 16, 18, and 19 (SEQ ID NOs: 90-92) (FIG. 3B).

FIG. 4: Synthesis of N^{a}-Fmoc-(N^{π}-allyl)-L-histidine-OH. (a) MeOH, HOBt, EDC, DCM, rt, ovn 73%; (b) i. Tf₂O (1.1 equiv.), DIPEA (1.2 equiv.), allylic alcohol (1.1 equiv.), −78° C. for 10 min and rt ovn; ii. TFA, TIPS, DCM, rt, 2 h, 70% for 2 steps; (c) HCl 2M, dioxane-water, reflux, ovn, 77%.

FIG. 5: Synthesis of Fmoc-Alnb-containing peptide. Linear precursor of compounds 15, 18 et 19.

FIG. 6: Synthesis of N^{a}-Fmoc-cypTyr(OR)—OH analogs. (a) phosphoric acid 85%, cyclopentanol, 100° C., ovn; (b) SOCl₂, MeOH, rt, ovn, 26% for 2 steps a and b; (c) Boc₂O, NaHCO₃, THF-water (1:1), rt, 1 h, 84%: (d) RBr, K₂CO₃, ACN, reflux, ovn, yield 17a (57%), 17b (54%), 17c (52%); (e) LiOH, THF-water (1:1), rt, 3 h, yield 113 (96%), 114 (100%), 115 (93%); (f) i. TFA-DCM (1:1), rt, 2 h; ii. Fmoc-Cl, NaHCO₃, THF-water (2:1), rt, 2 h, yield 116 (68%), 117 (41%), 118 (71%).

FIG. 7: N-terminal truncated analogs of 97, 15 and 16, namely compounds 20-23, and 24-28.

FIG. 8: Substitution of Nle11 (compound 29) by natural and unnatural amino acids to produce compounds 34-45.

FIGS. 9A-9B: Synthesis scheme for illustrative compounds of formula VI (e.g., compound 79, AM03-68 of Table III).

FIG. 10: Synthesis scheme for illustrative compounds of formula VII (e.g., compounds 75, 77-78 et 89-93 of Table III).

FIGS. 11A-IIB: Synthesis scheme for illustrative compounds of formula VIII (e.g., compounds 72-73 of Table III).

FIG. 12: Concentration-response curves of Ape13 macrocyclic analogs on the Gaᵢ₁, Ga₁₂ and b-arrestin2 pathways. Ligand-triggered engagement of the G protein Gαᵢₗ (A) ₘonitored using the BRET-based G protein dissociation assay (Galés et al., 2006). Ligand-induced recruitment of β-arrestin2 (B) using the BRET-based β-arrestin2 recruitment assay (Galés et al., 2006). Each set represents the mean of at least three independent experiments and expressed as the mean±SEM.

FIG. 13: In vivo pharmacokinetic profile of macrocycles 42 and 43 in male Sprague-Dawley rats (n=3). Compounds were administered intravenously at 3 mg/kg and their concentration in blood samples was quantified by LC/MS-MS.

FIG. 14: Hypotensive effects of compounds 15, 20, 29, 42 and 43 in anesthetized male Sprague-Dawley rats. Tracing depicted the change in blood pressure upon receipt of a bolus (i.v.) of compounds 15, 20, 29, 39, 42 and 43 at two doses (65 nmol/kg and 19.6 nmol/kg) or Ape13 via the jugular vein (n=4-6 per group).

FIGS. 15A-15B: Effect of macrocycles 42 and 43 on left ventricular fractional shortening (FIG. 15A) and cardiac output (FIG. 15B) (n=5 for all groups). *p<0.05, **p<0,01 vs. time-matched NS (not stimulated) using one way ANOVA test.

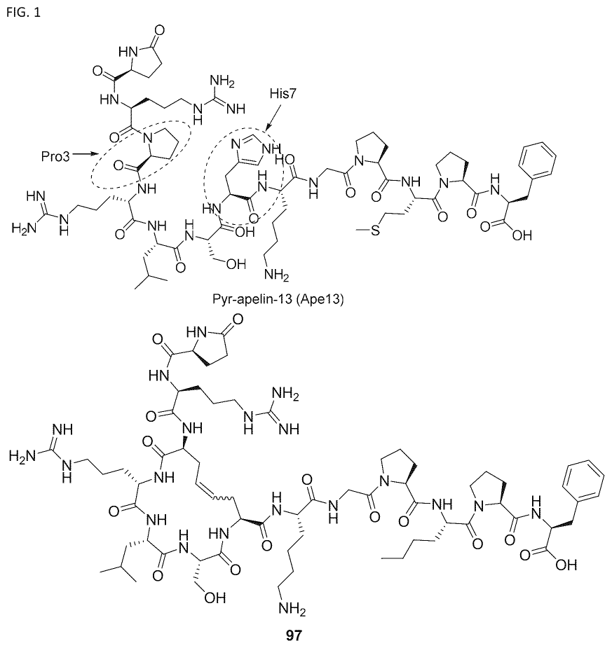

### DESCRIPTION OF ILLUSTRATIVE EMBODIMENTS

### Compounds of the Present Disclosure

The present disclosure provides novel apelinergic compounds including apelin 13 analogues, apelin 17 analogues and elabela analogues.

Apelin is a peptide hormone acting as the endogenous ligand of the class A G protein-coupled APJ receptor (Tatemoto et al., 1998; O'Dowd et al., 1993; and Read et al., 2019). As a GPCR target, the APJ receptor is known to couple to distinct G proteins, such as Gαᵢ, which primarily inhibits the cAMP-dependent pathway by inhibiting adenylate cyclase activity (Masri et al. 2006; and Habata et al., 1999). The APJ receptor also signals through the recruitment of β-arrestins, which has been associated with receptor desensitization (Besserer-Offroy et al., 2018; et Gurevich et al., 2019) The β-arrestin pathway is also known to couple with various effectors and to initiate downstream signaling on its own. (Gurevich et al., 2019; and Reiter et al., 2012).

Apelin and elabela are the two endogenous peptide ligands of APJ and possess similar binding potency and signaling profiles, despite very different primary sequences (Chng et al., 2013; Pauli et al., 2014; and Murza et al., 2016). Apelin exists in several isoforms: apelin-36, apelin-17, apelin-13, [Pyr¹]-apelin-13 and [Pyr¹]-apelin-13₍₁₋₁₂₎. Among them, [Pyr¹]-apelin-13 (Ape13) is the predominant isoform circulating in human plasma and heart tissue. (Tatemoto et al., 1998; Maguire et al., 2009; Yang et al., 2017; Nyimanu et al., 2019; Zhen et al., 2013).

### Compounds of the Present Disclosure

In specific embodiments, macrocyclic compounds of the present disclosure are developed from the cyclization of a synthetic peptide (generally made from natural and/or non-natural amino acids) derived from Apelin-13 (PyrRPRLSHKGPMPF (SEQ ID NO: 47)), Apelin-17 (KFRRQRPRLSHKGPMPF (SEQ ID NO: 86)) or a fragment of Elabela (PyrRRCMPLHSRVPFP (SEQ ID NO: 85)).

In specific embodiments, the cyclisation of the peptide is a side chain to side chain cyclisation. In specific embodiments, the cyclisation of the synthetic peptide is achieved through a reaction of ring-closing metathesis of alkene (—C═C—) (or alkyne (—C≡C—)) groups at the end of each of the side chains of the N- and a central amino acid (or acid) moieties, the cyclisation resulting in a single carbon-carbon double bond (or single carbon-carbon triple bond if alkyne groups are used). The macrocycle may then further be modified to replace the double bond by a single bond through palladium-catalyzed hydrogenation. (see e.g., compound 13).

In other specific embodiments, the cyclisation of the peptide is achieved through a macrolactamisation reaction between an amine at the end of the side chain of one of the N-terminal amino acids and a carboxylic acid at the end of the side chain of the amino acid residue used to close the cycle or the reverse.

In a specific embodiment, compounds of the present disclosure are of any one formula I to VIII, or are stereoisomers or a mixture thereof, or pharmaceutically acceptable salts, esters or solvates thereof. In case of discrepancies herein between the name (list of residues) and structure (formula) mentioned herein for compounds of the disclosure or parts thereof, the structure (formula) shall prevail. In case of discrepancies herein between the compounds as described in the sequence listing and the name (list of residues) and/or structure (formula) mentioned herein for compounds of the disclosure or parts thereof, the name (list of residues) and/or structure (formula) shall prevail.

References herein to amino acids or acids that are part of molecules of the present disclosure should be understood to designate amino acid or acid residues. At least one of their ends is linked to another amino acid or acid to form e.g., a peptide bond thereby losing a hydroxy group and/or one hydrogen of an amine group. Hence, for example, an amino acid or acid listed in any one of the definitions of X1, X2, X3, X4, X5 and X6 should be understood to be the corresponding amino acid or acid residue.

Compounds of the present disclosure have a binding affinity (Ki binding (nM)) to APJ of less than 1000 nM; in specific embodiments, less than 900, 800, 700, 600, 500, 400, 300, 200, 100, 90, 80, 70, 60, 50, 40, 35, 30, 35, 20, 15, 10, 9, 8, 7, 6, 5, 4, 3, or 2 nM or less than 1 nM. In specific embodiments, compounds of the present disclosure are compounds of any one Formula I to VIII, or of Tables I to III having a binding affinity (Ki binding (nM)) to APJ of less than 1000 nM; in specific embodiments, less than 900, 800, 700, 600, 500, 400, 300, 200, 100, 90, 80, 70, 60, 50, 40, 35, 30, 35, 20, 15, 10, 9, 8, 7, 6, 5, 4, 3, or 2 nM or less than 1 nM.

Unless indicated otherwise, definitions for residues provided herein include L and D configurations.

Apelin 13 analogues

The present disclosure encompasses apelin 13 cyclic analogues such as those described in formula (I)-(IV).

In a specific embodiment, the apelin 13 cyclic analogue comprises or consists in the following formula (I):

X1-X2-Y-[X3-X4-X5-X6-X7-X8-X9-X10-X11-X12]-X13-X14-X15-X16-X17-X18,

wherein
X1 is absent, —(CH₂)q-CH₃ or —(CF₂)q-CF₃, wherein q is 0 to 11, or is any natural amino acid; or any synthetic amino acid, the side chain of which is H, -—(C1-C12)alkyl, —(CF₂)q-CF₃ wherein q is 0 to 11, —(C3-C8)heteroalkyl, a —(CH₂)p-(C3-C8)aryl, —(CH₂)p-(C3-C8)heteroaryl, a —(CH₂)p-(C3-C8)cycloalkyl, or a —(CH₂)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S, —(C1-C6)alkyl, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl, and wherein the heteroatom in the heteroalkyl, heteroaryl or heterocycloalkyl is a N, O or S. In a specific embodiment, it is Pyr, or absent;
X2 and X7 are each independently absent, or a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a specific embodiment, X2 and X7 are each independently absent, or an amino acid, the side chain of which is —CH₂—(CH₂)p-guanidine, —CH₂—(CH₂)p-NH₂, or —(CH2)p-imidazole wherein p is 0 to 4. In a specific embodiment, X2 and X7 are each independently Lys, Orn, Dab (2,4-diaminobutyric acid), Dap (2,3-diaminopropionic acid), Arg, hArg, His or absent. In a more specific embodiment, X2 and X7 are each independently absent, —CH₂—(CH₂)p-guanidine, or —CH₂—(CH₂)p-NH₂, wherein p is 0 to 4; or are each independently Arg or Lys;
Y is H, Ac, Ac—NH, —NH₂, guanidine or absent,
X3 and X12 close the ring and are identical or different and are aliphatic residues, alkenyl residues, acid residues or a natural or non-natural amino acid, or a derivative thereof, these moieties being optionally substituted. In specific embodiments, they are residues bearing a terminal alkene or free carboxylic or amine function. In specific embodiments, they are each independently Lys, Orn, Dab (2,4-diaminobutyric acid), Dap (2,3-diaminopropionic acid), Asp, Glu, AllylGly, Nα-allyl-arginine, Nπ-allyl-histidine, Nγ-allyl-Nγ-nosyl-α,γ-diamino-butanoic acid, Nγ-allyl-α,γ-diamino-butanoic acid, or Nγ-allyl-Nγ-methyl-α,γ-diamino-butanoic acid whereby the cycle is closed by an amide bridge or an alkene In specific embodiments, at least one or both of X3 and X12 are allylglycine. In specific embodiments, if X3 is allylglycine, X12 is not allylglycine.
X4, X5 and X6 are each independently Ser, Thr, Asn, Gln, Asn-(8-aminooctanoic), Trp-(8-aminooctanoic) or absent. In a specific embodiment, X4, X5 and X6 are each independently Thr, Asn, Asn-(8-aminooctanoic), Trp-(8-aminooctanoic) or absent. In another specific embodiment, they are all absent.
X8 is absent or is Gly, Phe, Leu, Ile, Ser, Pro, Aib, Sar, Oic, βAla, Hyp or Hyp(OBn). In a specific embodiment, it is absent or Pro.
X9 is any natural amino acid; or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S, —(C1-C6)alkyl, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a more specific embodiment, X9 is absent, or any natural or synthetic amino acid, the side chain of which is —CH₂—(CH₂)p-NH₂, —CH₂—(CH₂)p-guanidine, —CH₂—(CH₂)p-NH₂, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a more specific embodiment, X9 is an amino acid, the side chain of which is —CH₂—(CH₂)p-guanidine, —CH₂—(CH₂)p-NH₂, or —(CH2)p-imidazole, preferably —CH₂—(CH₂)p-guanidine, or —CH₂—(CH₂)p-NH₂, wherein p is 0 to 4. In a more specific embodiment, X9 is Nle, Lys, Orn, Dab (2,4-diaminobutyric acid), Dap (2,3-diaminopropionic acid), Arg, or hArg. In a more specific embodiment, X9 is Arg;
X10 is any natural amino acid, or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S or a (C1-C6)alkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a more specific embodiment, X10 is Leu, Nle, alpha-methylleucine, cycloleucine, tert-leucine, cyclohexylalanine, alpha-methylphenylalanine, Ala, Val, Ile. In a more specific embodiment, it is Leu;
X11 is absent, or is any natural amino acid; or any synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH2)p-NH2, —CH2—(CH2)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S, —(C1-C6)alkyl, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a more specific embodiment, X11 is Ser or absent;
X13 is a natural or synthetic amino acid, the side chain of which is —CH₂—(CH₂)p-NH₂, CH₂—(CH2)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment X13 is Lys, Orn, Dab, Dap, Arg, —CH₂—(CH₂)p-guanidine, wherein p is 0 to 4, or His. In a specific embodiment X13 is Lys.
X14 is Gly, Phe, Leu, Ile, Ser, Pro, Aib, Sar, Oic, βAla, Hyp or Hyp(OBn). In a specific embodiment, it is Gly;
X15 and X17 are each independently Pro, Aib, Sar, Oic, βAla, Hyp or Hyp(OBn). In a specific embodiment, X15 and X17 are each independently absent or Pro.
X16 is any natural amino acid; or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S, —(C1-C6)alkyl, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment, it is an amino acid, the side chain of which is —(CH₂)p-(C3-C8)alkyl, or —(CH₂)p-(C3-C8)aryl, wherein the aryl is optionally fused with one or two (C3-C8)aryl, and wherein the aryl is optionally substituted with one or more substituents, wherein each substituent is independently O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl. In a more specific embodiment, it is Nle, alpha-methylleucine, cycloleucine, tert-leucine, cyclohexylalanine, alpha-methylphenylalanine, Phe, Tic ((S)—N-Fmoc-tetrahydroisoquinoline-3-carboxylic acid), Tyr, 1Nal, 2Nal, TyrOBn, cypTyr(OBn), dcypTyr(OBn), cypTyr(OCyp), cypTyr(OPr), D-1Nal, D-2Nal, D-TyrOBn, or D-Tyr;
X18 is absent; is any natural amino acid; or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S, —(C1-C6)alkyl, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment, it is absent or is an amino acid, the side chain of which is a —(CH₂)p-(C3-C8)aryl, wherein the aryl is optionally substituted with one or more substituents, wherein each substituent is independently an halogen, amine, —OH, S or a —(C1-C6)alkyl. In another specific embodiment, it is Phe or an halogen substituted Phe. In another specific embodiment, it is absent. In another embodiment, it is Phe.

In a specific embodiment of compounds of Formula (I), when X3 is allylglycine, X12 is not allylglycine. In a specific embodiment of compounds of Formula (I), when X17 and X18 are absent, X16 is not Ala.In specific embodiments, compounds of formula (I) are any one of compounds 13-29, and 35-46 of Table I. In other specific embodiments, compounds of formula (I) are any one of compounds 13, 15-16, 18-20, 28, and 42-44 of Table I.

The present disclosure comprises compounds of Formula (I), wherein each of X1 to X18 are independently defined using any of the more general or more specific definitions provided above for these residues in Formula (I).

In another specific embodiment, the apelin 13 cyclic analogue comprises or consists in the following formula (II):

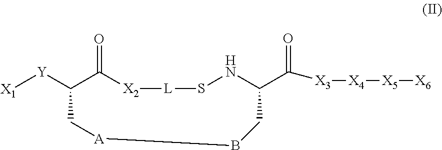

wherein X₁ is absent, or is X₇-X₈, wherein X₇ is —(CH₂)q-CH₃ or —(CF₂)q-CF₃ wherein q is 0 to 11, a natural amino acid, a synthetic amino acid, the side chain of which is H, a —(C1-C12)alkyl, —(CF₂)q-CF₃ wherein q is 0 to 11, —(C3-C8)heteroalkyl, a —(CH₂)p-(C3-C8)aryl, —(CH₂)p-(C3-C8)heteroaryl, —(CH₂)p-(C3-C8)cycloalkyl, or —(CH₂)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S, -(C1-C6)alkyl, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl, and wherein the heteroatom in the heteroalkyl, heteroaryl or heterocycloalkyl is a N, O or S. In a specific embodiment, X₇ is Pyr; and
X₈ is absent, or is a natural or synthetic amino acid, the side chain of which is —CH₂—(CH₂)p-NH₂, —CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a specific embodiment, X₈ is an amino acid, the side chain of which is —CH₂—(CH₂)p-guanidine, —CH₂—(CH₂)p-NH₂, or —(CH₂)p-imidazole, preferably —CH₂—(CH₂)p-guanidine, or —CH₂—(CH₂)p-NH₂, wherein p is 0 to 4. In a specific embodiment, X₈ is Lys, Orn, Dab (2,4-diaminobutyric acid), Dap (2,3-diaminopropionic acid), Arg, hArg, His or absent, preferably Lys, Orn, Dab (2,4-diaminobutyric acid), Dap (2,3-diaminopropionic acid), Arg, or hArg. In a more specific embodiment, X₈ is Arg.
Y is absent, NH₂—, Ac—NH—, guanidine, or H;
A is —(CH₂)n-; —(CH₂)nNH═C(NH₂)N—CH₂—CH═CH— (preferably Nα-allyl-arginine), wherein n is 2, 3 or 4; or —CH═CH—(CH₂)m, wherein m is 0, 1 or 2 (preferably allyl-glycine);
B is absent or

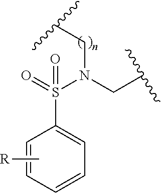

wherein R is O, P, m-alkyl, halogen or nitro and n is 1, 2, or 3;

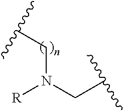

wherein R is H, C3-C7 alkyl, benzyl or arylalkyle and n is 1, 2 or 3;

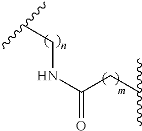

wherein n is 1, 2, 3 or 4 and m is 0 or 1; or

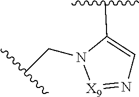

wherein X₉ is CH or N.

X₂ and X₃ are each independently absent, or a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a specific embodiment, X₂ and X₃ are each independently an amino acid, the side chain of which is —CH₂—(CH₂)p-guanidine, —CH₂—(CH₂)p-NH₂, or —(CH₂)p-imidazole, preferably —CH₂—(CH₂)p-guanidine, or —CH₂—(CH₂)p-NH₂, wherein p is 0 to 4. In a specific embodiment, X₂ and X₃ are each independently Lys, Orn, Dab (2,4-diaminobutyric acid), Dap (2,3-diaminopropionic acid), Arg, hArg, His, Nle, alpha-methylleucine, cycloleucine, tert-leucine, cyclohexylalanine (e.g., (3-cyclohexyl-L-alanine) or alpha-methylphenylalanine. In specific embodiments, X₂ and X₃ are each independently Lys, Arg, hArg, Nle, Leu, Phe, or Cha. In a more specific embodiment, X₂ and X₃ are each independently Arg or Lys.

X₄ is a natural or non-natural amino acid having a positively charged or uncharged sidechain. In specific embodiments, X₄ is Gly, Phe, Leu, Ile, Ser, Aib, Pro, Sar, Oic, βAla, Hyp or Hyp(OBn). In a specific embodiment, it is Gly;

X₈ is Gly, Phe, Leu, Ile, Ser, Aib, Pro, Sar, Oic, βAla, Hyp or Hyp(OBn). In a specific embodiment, it is Pro;

X₆ is X₁₀-X₁₁-X₁₂, wherein
X₁₀ is any natural amino acid; or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S, -(C1-C6)alkyl, —O—(C1-C6)alkyl, —(CH₂)p′—(C3-C8)aryl, —O—(CH₂)p′—(C3-C8)aryl, —(C3-C8)cycloalkyl, or —O—(C3-C8)cycloalkyl, wherein p′ is 0 to 5; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment, X₁₀ is an amino acid, the side chain of which is —(CH₂)p-(C3-C8)alkyl, or —(CH₂)p-(C3-C8)aryl, wherein p is 0 to 5, wherein the aryl is optionally fused with one or two (C3-C8)aryl, and wherein the aryl is optionally substituted with one or more substituents, wherein each substituent is independently, —OH, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl, wherein p is 0 to 5. In a specific embodiment, it is not Ala. In a more specific embodiment, X₁₀ is Nle, alpha-methylleucine, cycloleucine, tert-leucine, cyclohexylalanine (e.g., (3-cyclohexyl-L-alanine), alpha-methylphenylalanine, Phe, Tic ((S)—N-Fmoc-tetrahydroisoquinoline-3-carboxylic acid), Tyr, 1Nal, 2Nal, TyrOBn, cypTyr(OBn), dcypTyr(OBn), cypTyr(OCyp), cypTyr(OPr), D-1Nal, D-2Nal, D-TyrOBn, or D-Tyr;
X₁₁ is absent or Gly, Phe, Leu, Ile, Ser, Aib, Pro, Sar, Oic, βAla, Hyp or Hyp(OBn). In a specific embodiment, it is absent or Pro; and
X₁₂ is absent or Phe.

In specific embodiments, compounds of formula (II) are any one of compounds 13-25, 27-29, 35-37 and 42-45 of Table I. In other specific embodiments, compounds of formula (II) are any one of compounds 13, 15-16, 18-20, 28, and 42-44 of Table I.

In a specific embodiment, X₁ is Pyr-Arg, Y is —NH—, A is-CH₂—CH₂—, B is absent, X₂ is Arg, X₃ Lys, X₄ is Gly, X₈ is Pro, and X₆ is Nle-Pro-Phe.

In another specific embodiment, X₁ is Pyr-Arg, Y is —NH—, A is —CH═CH—, B is

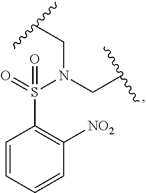 [SMILES: *CN(C*)S(=O)(=O)c1ccccc1[N+](=O)[O-]]

X₂ is Arg, X₃ is Lys, X₄ is Gly, X₅ is Pro, X₆ is Nle-Pro-Phe].

In another specific embodiment, X₁ is Pyr-Arg, Y is —NH—, A is —CH═CH—, B is

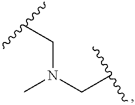 [SMILES: *CN(C)C*]

X₂ is Arg, X₃ is Lys, X₄ is Gly, X₈ is Pro, X₆ is Nle-Pro-Phe.

In another specific embodiment, X₁ is Pyr-Arg, Y is —NH—, A is —CH₂—CH₂—, B is

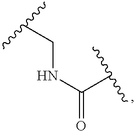 [SMILES: *CNC(*)=O]

### X2 is Arg, X3 is Lys, X4 is Gly, X5 is Pro, and X6 is Nle-Pro-Phe.

In another specific embodiment, X1 is Pyr-Arg, Y is —NH—, A is —CH═CH—, B is

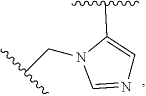 [SMILES: *Cn1cncc1*]

### X2 is Arg, X3 is Lys, X4 is Gly, X5 is Pro, X6 is Nle-Pro-Phe.

In another specific embodiment, X1 is H, Y is —NH—, A is —CH═CH—, B is absent, X2 is Arg, X3 is Lys, X4 is Gly, X5 is Pro, X6 is Nle-Pro-Phe.

In another specific embodiment, X1 is absent, Y is —H, A is —CH═CH—, B is absent, X2 is Arg, X3 is Lys, X4 is Gly, X5 is Pro, X6 is Nle.

In another specific embodiment, X1 is H, Y is —NH—, A is —CH═CH—, B is absent, X2 is Nle, X3 is Lys, X4 is Gly, X5 is Pro, X6 is Nle.

In another specific embodiment, X1 is H, Y is —NH—, A is —CH═CH—, B is absent, X2 is Arg, X3 is Lys, X4 is Gly, X5 is Pro, X6 is Nle.

In another specific embodiment, X1 is H, Y is —NH—, A is —CH═CH—, B is absent, X2 is Arg, X3 is Lys, X4 is Gly, X5 is Pro, X6 is D-1Nal.

In another specific embodiment, X1 is H, Y is —NH—, A is —CH═CH—, B is absent, X2 is Arg, X3 is Lys, X4 is Gly, X5 is Pro, X6 is D-2Nal.

In a specific embodiment of compounds of Formula (II), when A is allylglycine, B is not allylglycine.

The present disclosure comprises compounds of Formula (II), wherein each of the variables X1, X2, X3, X4, X5, X6, Y, A and B are independently defined using any of the more general or more specific definitions provided above for these residues in Formula (II).

In another specific embodiment, the apelin 13 cyclic analogue comprises or consists in the following formula (III):

X1-[X2-X3-X4-X5-X6-X7]-X8-X9-X10-X11-X12-X13-X14-X15,

wherein X1 is absent, —(CH₂)q-CH₃ or —(CF₂)q-CF₃ wherein q is 0 to 11, a natural amino acid, a synthetic amino acid, the side chain of which is H, a —(C1-C12)alkyl, —(CF₂)q-CF₃ wherein q is 0 to 11, —(C3-C8)heteroalkyl, a —(CH₂)p-(C3-C8)aryl, —(CH₂)p-(C3-C8)heteroaryl, —(CH₂)p-(C3-C8)cycloalkyl, or —(CH₂)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S, —(C1-C6)alkyl, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl, and wherein the heteroatom in the heteroalkyl, heteroaryl or heterocycloalkyl is a N, O or S. In a specific embodiment, X1 is Pyr, —(CH₂)q-CH₃ or —(CF2)q-CF₃ wherein q is 0 to 11;

X2 and X7 close the ring and are identical or different and are aliphatic residues, alkenyl residues, acid residues or a natural or non-natural amino acid, or a derivative thereof, these moieties being optionally substituted. In specific embodiments, they are residues bearing a terminal alkene or free carboxylic or amine function. In specific embodiments, they are each independently Lys, Orn, Dab (2,4-diaminobutyric acid), Dap (2,3-diaminopropionic acid), Asp, Glu, AllylGly, Nα-allyl-arginine, Nπ-allyl-histidine, Nγ-allyl-Nγ-nosyl-α,γ-diamino-butanoic acid, Nγ-allyl-α,γ-diamino-butanoic acid, or Nγ-allyl-Nγ-methyl-α,γ-diamino-butanoic acid whereby the cycle is closed by an amide bridge or an alkene. In specific embodiments, at least one or both of X2 and X7 are allylglycine;

X3 is Gly, Phe, Leu, Ile, Ser, Aib, Pro, Sar, Oic, βAla, Hyp or Hyp(OBn). In a specific embodiment, it is absent or Pro;

X4 is a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a specific embodiment, X4 is an amino acid, the side chain of which is —CH₂—(CH₂)p-guanidine, —CH₂—(CH₂)p-NH₂, or —(CH₂)p-imidazole, preferably —CH₂—(CH₂)p-guanidine or —CH₂—(CH₂)p-NH₂, wherein p is 0 to 4. In a specific embodiment, X4 is Lys, Orn, Dab (2,4-diaminobutyric acid), Dap (2,3-diaminopropionic acid), Arg, or His. In a more specific embodiment, X4 is Arg or Lys. In a more specific embodiment, X4 is Arg.

X5 and X6 are each independently absent or any natural amino acid, or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S or a (C1-C6)alkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment, X5 is Leu and/or X6 is Ser;

X8 is absent or is any natural amino acid, or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S or a (C1-C6)alkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment, X8 is Ser;

X9 is absent or is a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably. In a specific embodiment, X9 is absent, or an amino acid, the side chain of which is —CH₂—(CH₂)p-guanidine, —CH₂—(CH₂)p-NH₂, or —(CH₂)p-imidazole, preferably —(CH₂)p-imidazole, wherein p is 0 to 4. In a specific embodiment, X9 is absent, Lys, Orn, Dab (2,4-diaminobutyric acid), Dap (2,3-diaminopropionic acid), Arg, or His. In a more specific embodiment, X9 is absent or His;

X10 is a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a more specific embodiment, X10 is an amino acid, the side chain of which is —CH₂—(CH₂)p-guanidine, —CH₂—(CH₂)p-NH₂, or —(CH₂)p-imidazole, preferably —CH₂—(CH₂)p-guanidine, or —CH₂—(CH₂)p-NH₂, wherein p is 0 to 4. In a specific embodiment, X10 is Lys, Orn, Dab (2,4-diaminobutyric acid), Dap (2,3-diaminopropionic acid), Arg, hArg, or His. In a more specific embodiment, X10 is Lys;

X11, X12 and X14 are each independently Gly, Phe, Leu, Ile, Ser, Pro, Aib, Sar, Oic, βAla, Hyp or Hyp(OBn). In a specific embodiment, X11 is Gly. In a specific embodiment, X12 and/or X14 are Pro;

X13 is Nle, alpha-methylleucine, cycloleucine, tert-leucine, cyclohexylalanine (e.g., (3-cyclohexyl-L-alanine), alpha-methylphenylalanine, preferably Nle;

X15 is an amino acid, the side chain of which is —(CH₂)p-(C3-C8)aryl, wherein the aryl is optionally substituted with one or more substituents, wherein each substituent is independently an halogen, amine, —OH, S or a (C1-C6)alkyl. In specific embodiments, X15 is a Phe or a halogen substituted Phe. In specific embodiments, X15 is a Phe.

In specific embodiments, compounds of Formula (III) are any one of compounds 3 and 4 of Table I.

The present disclosure comprises compounds of Formula (III), wherein each of X1 to X15 are independently defined using any of the more general or more specific definitions provided above for these residues in Formula (III).

In another specific embodiment, the apelin 13 cyclic analogue comprises or consists in the following formula (IV):

X1-X2-X3-X4-X5-X6-X7-X8-X9-X10-[X11-X12-X13-X14-X15],

wherein Xaa1 is —(CH₂)q-CH₃ or —(CF₂)q-CF₃ wherein q is 0 to 11, a natural amino acid, a synthetic amino acid, the side chain of which is H, a —(C1-C12)alkyl, —(CF₂)q-CF₃ wherein q is 0 to 11, —(C3-C8)heteroalkyl, a —(CH₂)p-(C3-C8)aryl, —(CH₂)p-(C3-C8)heteroaryl, —(CH₂)p-(C3-C8)cycloalkyl, or —(CH₂)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S, —(C1-C6)alkyl, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl, and wherein the heteroatom in the heteroalkyl, heteroaryl or heterocycloalkyl is a N, O or S. In a specific embodiment, X1 is Pyr, —(CH₂)q-CH₃ or —(CF₂)q-CF₃ wherein q is 0 to 11. In a more specific embodiment, it is Pyr;

X2, X4, X7 and X8 are each independently a natural or synthetic amino acid, the side chain of which is —CH₂—(CH₂)p-NH₂, —CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a specific embodiment, X2, X4, X7 and X8 are each independently an amino acid, the side chain of which is —CH₂—(CH₂)p-guanidine, —CH₂—(CH₂)p-NH₂, or —(CH₂)p-imidazole wherein p is 0 to 4. In a specific embodiment, X2, X4, X7 and X8 are each independently Lys, Orn, Dab (2,4-diaminobutyric acid), Dap (2,3-diaminopropionic acid), Arg, hArg, or His. In a more specific embodiment, X2, X4, X7 and X8 are each independently Arg, His or Lys. In a more specific embodiment, X2, X4, and X8 are each independently an amino acid, the side chain of which is —CH₂—(CH₂)p-guanidine, or —CH₂—(CH₂)p-NH₂, wherein p is 0 to 4; or are each independently Arg or Lys; and/or X7 is an amino acid, the side chain of which is-(CH₂)p-imidazole wherein p is 0 to 4; or is His;

X3 is Gly, Phe, Leu, Ile, Ser, Pro, Aib, Sar, Oic, βAla, Hyp or Hyp(OBn). In a specific embodiment, X3 is Pro;

X5 and X6 are each independently any natural amino acid, or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S or a (C1-C6)alkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment X5 is Leu; and/or X6 is Ser;

X9 is absent, or is a natural or non-natural amino acid having a positively charged or uncharged sidechain. In specific embodiments, X4 is Gly, Phe, Leu, Ile, Ser, Aib, Pro, Sar, Oic, βAla, Hyp or Hyp(OBn). In a specific embodiment, it is Gly;

X10 and X12 are each independently absent or Gly, Phe, Leu, Ile, Ser, Pro, Aib, Sar, Oic, βAla, Hyp or Hyp(OBn). In a more specific embodiment, X10 is not absent. In a specific embodiment, X10 and X12 are each independently absent or Pro;

X11 and X15 close the ring and are identical or different and are aliphatic residues, alkenyl residues, acid residues or a natural or non-natural amino acid, or a derivative thereof, these moieties being optionally substituted. In specific embodiments, they are residues bearing a terminal alkene or free carboxylic or amine function. In specific embodiments, they are each independently Lys, Orn, Dab (2,4-diaminobutyric acid), Dap (2,3-diaminopropionic acid), Asp, Glu, AllylGly, Nα-allyl-arginine, Nπ-allyl-histidine, Nγ-allyl-NT-nosyl-α,γ-diamino-butanoic acid, Nγ-allyl-α,γ-diamino-butanoic acid, or Nγ-allyl-Nγ-methyl-α,γ-diamino-butanoic acid whereby the cycle is closed by an amide bridge or an alkene. In specific embodiments, X12 and X15 are independently allylglycine or D-allylglycine;

X13 is absent or is any natural amino acid, or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S or a (C1-C6)alkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In specific embodiments, X13 is Nle, alpha-methylleucine, cycloleucine, tert-leucine, cyclohexylalanine (e.g., (3-cyclohexyl-L-alanine), alpha-methylphenylalanine, preferably Nle.

X14 is Gly, Phe, Leu, Ile, Ser, Pro, Aib, Sar, Oic, βAla, Hyp or Hyp(OBn). In a specific embodiment, X14 is Pro.

In a specific embodiment of compounds of formula IV, X9 and X10 are not absent. In a specific embodiment, if X11 is allylglycine, X15 is not allylglycine. In specific embodiments, X11 is not B1 or B2.

In specific embodiments, compounds of formula (IV) are any one of compounds 9-12 of Table I.

In other specific embodiments, the compound of formula (I) is compound 12 of Table I.

The present disclosure comprises compounds of Formula (IV), wherein each of X1 to X15 are independently defined using any of the more general or more specific definitions provided above for these residues in Formula (IV).

In specific embodiments of formula (I) to (IV), at least 4 (or at least 5, 6, 7, 8, 9, or 10) of the residues (e.g., residues a positions Xn defined above, wherein n is 1 to 18 for formula (I), 1 to 12 for formula (II) or 1 to 18 for formula (III) and (IV)), other than residues closing the cycle which differ from the corresponding residues in AP-13 (SEQ ID NO: 47), correspond to those in AP-13 (SEQ ID NO: 47). For example, compound 3 has at least 10 residues corresponding to those in AP-13 (SEQ ID NO: 47).

In other specific embodiments, compounds of the present disclosure correspond to macrocyclic analogs of Ap13 PyrRPRLSHKGPMPF (SEQ ID NO: 47), wherein the compounds vary from Ap13 by at least two substitutions at the positions closing the cycle, and by at least one (or 2, 3, 4, 5, 6, 7 or 8) further substitution(s), deletion(s) and/or insertion(s). These substitutions, deletions and/or insertions are defined in the various Xn of formula (I) to (IV) above. The correspondence between these Xn and Ap13 is shown in Table A below, wherein the “[” and “]” symbols are used to denote the positions of the ring closure residues in formula (I) to (IV) and compounds of the disclosure satisfying these formula.

TABLE A

| | Ap13 | | | | | | | | | | | | | | | | | | | | | | |
|---|---|---|---|---|---|---|---|---|---|---|---|---|---|---|---|---|---|---|---|---|---|---|---|
| | Pyr | R | | P | | | | | | R | L | S | H | | | K | | | G | P | M | P | F |
| | SEQ ID NO: 47 | | | | | | | | | | | | | | | | | | | | | | |
| | 1 | 2 | | 3 | | | | | | 4 | 5 | 6 | 7 | | | 8 | | | 9 | 10 | 11 | 12 | 13 |
| Formula | X1 | X2 | Y | [X3 | X4 | X5 | X6 | X7 | X8 | X9 | X10 | X11 | X12] | | | X13 | | | X14 | X15 | X16 | X17 | X18 |
| (I) | | | | | | | | | | | | | | | | | | | | | | | |
| Formula | X7 | X8 | Y | | | | | | | X2 | L | S | | | | X3 | | | X4 | X5 | X10 | X11 | X12 |
| (II) | | | | | | | | | | | | | | | | | | | | | | | |
| Formula | X1 | | [X2 | X3 | | | | | | X4 | X5 | X6 | X7] | X8 | X9 | X10 | | | X11 | X12 | X13 | X14 | X15 |
| (III) | | | | | | | | | | | | | | | | | | | | | | | |
| Formula | X1 | X2 | | X3 | | | | | | X4 | X5 | X6 | X7 | | | X8 | X9 | X10 | [X11 | X12 | X13 | X14 | X15] |
| (IV) | | | | | | | | | | | | | | | | | | | | | | | |

TABLE B

| | Ap-17 | | | | | | | | | | | | | | | | |
|---|---|---|---|---|---|---|---|---|---|---|---|---|---|---|---|---|---|
| | K | F | R | R | Q | R | P | R | L | S | H | K | G | P | M | P | F |
| | SEQ ID NO: 86 | | | | | | | | | | | | | | | | |
| | 1 | 2 | 3 | 4 | 5 | 6 | 7 | 8 | 9 | 10 | 11 | 12 | 13 | 14 | 15 | 16 | 17 |
| | Ap13 | | | | | | | | | | | | | | | | |
| | | | | | Pyr | R | P | R | L | S | H | K | G | P | M | P | F |
| | SEQ ID NO: 47 | | | | | | | | | | | | | | | | |
| | | | | | 1 | 2 | 3 | 4 | 5 | 6 | 7 | 8 | 9 | 10 | 11 | 12 | 13 |
| Formula (V) | X1 | X2 | X3 | X4 | X5 | X6 | X7 | X8 | X9 | [X10 | X11 | X12 | X13] | X14 | X15 | X16 | X17 |

TABLE C

| | ELA(19-32) | | | | | | | | | | | | | | | | | | |
|---|---|---|---|---|---|---|---|---|---|---|---|---|---|---|---|---|---|---|---|
| | Pyr | R | R | C | | M | | P | L | | H | | | S | R | V | P | F | P |
| | SEQ ID NO: 85 | | | | | | | | | | | | | | | | | | |
| | 1 | 2 | 3 | 4 | | 5 | | 6 | 7 | | 8 | | | 9 | 10 | 11 | 12 | 13 | 14 |
| Formula (VI) | [X1′ | X2′ | X3′ | X4′] | | X5′ | X6′ | [C | X7′ | X8′ | X9′ | | | C] | X10′ | X11′ | X12′ | X13′ | X14′ |
| Formula (VII) | X1′ | X2′ | X3′ | X4′ | [X5′ | X6′ | | X7′ | X8′ | | X9′ | X10′] | X11′ | X12′ | X13′ | X14′ | X15′ | X16′ | X17′ |
| Formula (VIII) | [X1′ | X2′ | X3′ | X4′] | | X5′ | | X6′ | X7′ | | X8′ | | | X9′ | X10′ | X11′ | X12′ | X13′ | X14′ |

### Apelin 17 Analogues

In a specific embodiment, the apelin 17 cyclic analogues comprise or consist in the following formula (V):

X1-X2-X3-X4-X5-X6-X7-X8-X9-[X10-X11-X12-X13]-X14-X15-X16-X17,

wherein X1 is a natural or synthetic amino acid, the side chain of which is —R—CH₂—(CH₂)p-NH₂, —R—CH₂—(CH₂)p-guanidine, —R—(CH₂)p-(C3-C8)cycloalkyl, —R—(CH₂)p-(C3-C8)heterocycloalkyl, —R—(CH₂)p-(C3-C8)aryl, or —R—(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N, wherein R is absent or is an acetyl. In a specific embodiment, X1 is an amino acid, the side chain of which is —CH₂—(CH₂)p-guanidine, —CH₂—(CH₂)p-NH₂, or —(CH₂)p-imidazole, preferably —CH₂—(CH₂)p-guanidine, or —CH₂—(CH₂)p-NH₂, wherein p is 0 to 4. In a specific embodiment, X1 is R-Lys, R-Orn, R-Dab (2,4-diaminobutyric acid), R-Dap (2,3-diaminopropionic acid), R-Arg, R-hArg, of R-His, wherein R is absent or acetyl. In a specific embodiment, X1 is Ac-Lys;
X2 is Phe;
X3, X4, X6, X8, X11 and X12 are each independently a natural or synthetic amino acid, the side chain of which is —(CH₂)p-(C3-C8)alkyl, —CH₂—(CH₂)p-NH₂, —CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a specific embodiment, X2, X4, X7, X8, X11 and X12 are each independently an amino acid, the side chain of which is —CH₂—(CH₂)p-guanidine, —CH₂—(CH₂)p-NH₂, or —(CH₂)p-imidazole, preferably —CH₂—(CH₂)p-guanidine, or —CH₂—(CH₂)p-NH₂, wherein p is 0 to 4. In a specific embodiment, X3, X4, X6, X8, X11 and X12 are each independently Lys, Orn, Dab (2,4-diaminobutyric acid), Dap (2,3-diaminopropionic acid), Arg, hArg, or His. In a specific embodiment, X3, X4, X6, X8, X11 and X12 are each independently an amino acid, the side chain of which is —CH₂—(CH₂)p-guanidine, or —CH₂—(CH₂)p-NH₂, wherein p is 0 to 4; or are each independently Arg, His or Lys. In a specific embodiment, X3, X4, X6 and X8 are each are each independently an amino acid, the side chain of which is —CH₂—(CH₂)p-guanidine wherein p is 0 to 4; or are Arg; and/or X11 is an amino acid, the side chain of which is-(CH₂)p-imidazole wherein p is 0 to 4; or is His; and/or X12 is an amino acid, the side chain of which is —CH₂—(CH₂)p-NH₂, wherein p is 0 to 4; or X12 is Lys;
X5 is Gln;
X7, X14 and X16 are each independently Gly, Phe, Leu, Ile, Ser, Pro, Aib, Sar, Oic, βAla, Hyp or Hyp(OBn). In a specific embodiment, at least one, 2 or all 3 of X7, X14 and X16 are Pro;
X9 and X15 are each independently any natural amino acid, or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S or a (C1-C6)alkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a more specific embodiment, X9 and X15 are each independently a natural or synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5. In a more specific embodiment, X9 and X15 are each independently a natural or synthetic amino acid, the side chain of which is —(C3-C6)alkyl. In another specific embodiment, X9 and X15 are each independently Nle, Leu, Ala, alpha-methylleucine, cycloleucine, tert-leucine, cyclohexylalanine (e.g., (3-cyclohexyl-L-alanine), alpha-methylphenylalanine, In a more specific embodiment, X9 is Leu; and/or X15 is Ala or Nle;
X10 and X13 close the ring and are identical or different and are aliphatic residues, alkenyl residues, acid residues or a natural or non-natural amino acid, or a derivative thereof, these moieties being optionally substituted. In specific embodiments, they are residues bearing a terminal alkene or free carboxylic or amine function. In specific embodiments, they are each independently Lys, Orn, Dab (2,4-diaminobutyric acid), Dap (2,3-diaminopropionic acid), Asp, Glu, AllylGly, Nα-allyl-arginine, Nπ-allyl-histidine, Nγ-allyl-Nγ-nosyl-α,γ-diamino-butanoic acid, Nγ-allyl-α,γ-diamino-butanoic acid, or Nγ-allyl-Nγ-methyl-α,γ-diamino-butanoic acid whereby the cycle is closed by an amide bridge or an alkene. In specific embodiments, one of X10 and X13 is Glu and the other is Lys;
X17 is any natural amino acid or any synthetic amino acid, the side chain of which is a —(CH₂)p-(C3-C8)aryl, —(CH₂)p-(C3-C8)heteroaryl, a —(CH₂)p-(C3-C8)cycloalkyl, a —(CH₂)p-(C3-C8)heterocycloalkyl or a —(CH₂)p-CONH-aryl; or is —(CH₂)p-CON(aryl)(alkylaryl), wherein p is 0 to 5, and wherein the heteroaryl, aryl, cycloalkyl and heterocycloalkyl is optionally substituted with one or more substituents; wherein each substituent is independently e.g., an halogen, amine, —(C3-C8)aryl, —(C3-C8)cycloalkyl, —O—(C3-C8)aryl, —O—(C3-C8)cycloalkyl, —OH, S, a —(C1-C6)alkyl, —O—(C1-C6)alkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; wherein the heteroatom in the heteroalkyl, heteroaryl or heterocycloalkyl is a N, O or S; In a specific embodiment, X17 is any natural amino acid or any synthetic amino acid, the side chain of which is a —(CH₂)p-(C3-C8)aryl wherein p is 0 to 5, substituted or not. In a specific embodiment, X17 is Phe, cypTyrOBn, cypTyr, dcypTyrOBn, TyrOBn, B1, B2, B3 or B4.

In specific embodiments, compounds of formula (V) are any one of compounds 62-70 of Table II.

The present disclosure comprises compounds of Formula (V), wherein each of X1 to X17 are independently defined using any of the more general or more specific definitions provided above for these residues in formula (V).

In specific embodiments of formula (V), at least 4 (or at least 5, 6, 7, 8, 9, 10, 11 or 12) of the residues at positions Xn defined above, wherein n is 1 to 12 and 14 to 17 (i.e. other than residues X10 and X13 closing the cycle which differ from the corresponding residues in AP-17 (SEQ ID NO: 86)), correspond to those in AP-17 (SEQ ID NO: 86). For example, compound 62 has 11 residues corresponding to those in AP-17 (SEQ ID NO: 86). In other specific embodiments of formula (V), except for residues closing the cycle which differ from the corresponding residues in AP-13 (SEQ ID NO: 47), at least 4 (or at least 5, 6, 7, 8, or 9) of the residues correspond to those in AP-13 (SEQ ID NO: 47). For example, compound 62 has 8 residues corresponding to those in AP-13 (SEQ ID NO: 47).

In other specific embodiments, compounds of the present disclosure correspond to macrocyclic analogs of Ap17 KFRRQRPRLSHKGPMPF (SEQ ID NO: 86), wherein the compounds vary from Ap17 by at least two substitutions at positions closing the cycle, and at least one (or 2, 3, 4, 5, 6, 7, 8, 9 or 10) further substitution(s), deletion(s) and/or insertion(s). These substitutions, deletions and/or insertions are defined in the various Xn of formula (V) above. The correspondence between these Xn and Ap13 and A17 is shown in Table B above, wherein the “[” and “]” symbols are used to denote the positions of the ring closure residues in formula (V) and in compounds of the disclosure satisfying this formula.

### Elabela Cyclic Analogues

The present disclosure also encompasses Elabela cyclic analogues such as those described in any one of formula (VI) to (VIII).

In a specific embodiment, the elabela cyclic analogue comprises or consists in the following formula (VI):

c[X1′-X2′-X3′-X4′]c-X5′-X6′-c[C-X7′-X8′-X9′—C]c-X10′-X11′-X12′-X13′-X14′,

wherein:
X1′ and X4′ close the ring and are identical or different and are aliphatic residues, alkenyl residues, acid residues or a natural or non-natural amino acid, or a derivative thereof, these moieties being optionally substituted. In specific embodiments, they are residues bearing a terminal alkene or free carboxylic or amine function. In specific embodiments, they are each independently Lys, Orn, Dab (2,4-diaminobutyric acid), Dap (2,3-diaminopropionic acid), Asp, Glu, AllylGly, Nα-allyl-arginine, Nπ-allyl-histidine, Nγ-allyl-Nγ-nosyl-α,γ-diamino-butanoic acid, Nγ-allyl-α,γ-diamino-butanoic acid, or Nγ-allyl-Nγ-methyl-α,γ-diamino-butanoic acid whereby the cycle is closed by an amide bridge or an alkene. In specific embodiments, one of X1′ and X4′ is Glu and the other is Lys. In a specific embodiment, they are Lys and Glu or Glu and Lys;
X2′, X3′ and X9′ and X10′ are each independently a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment, X2′, X3′, X9′ and X10′ are each independently absent or an amino acid, the side chain of which is —CH₂—(CH₂)p-guanidine, —CH₂—(CH₂)p-NH₂, or —(CH₂)p-imidazole, preferably —CH₂—(CH₂)p-guanidine, or —CH₂—(CH₂)p-NH₂, wherein p is 0 to 4. In a specific embodiment, X2′, X3′, X9′ and X10′ are each independently Lys, Orn, Dab (2,4-diaminobutyric acid), Dap (2,3-diaminopropionic acid), Arg, His or absent. In a more specific embodiment, X2′ and X3′ are each independently —CH₂—(CH₂)p-guanidine, wherein p is 0 to 4; or Arg; and/or X9′ is —(CH₂)p-imidazole, wherein p is 0 to 4 or His; and/or X10′—(CH₂)p-(C3-C8)aryl. —CH₂—(CH₂)p-guanidine, or —CH₂—(CH₂)p-NH₂, wherein p is 0 to 4 or is Arg, Orn, Lys, or 4-aminomethyl-phenylalanine;
X12′ and X14′ are each independently Gly, Phe, Leu, Ile, Ser, Pro, Aib, Sar, Oic, βAla, Hyp or Hyp(OBn). In a specific embodiment, X12′ and/or X14′ is/are Pro;
X5′, X7′, and X11′, are each independently any natural amino acid; or any synthetic amino acid, the side chain of which is H, a —(CH₂)p-(C3-C8)alkyl, —(CH₂)p-(C3-C8)heteroalkyl, a —(CH₂)p-(C3-C8)aryl, —(CH₂)p-(C3-C8)heteroaryl, a —(CH₂)p-(C3-C8)cycloalkyl, or a —(CH₂)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S or a (C1-C6)alkyl, and wherein the heteroatom in the heteroalkyl, heteroaryl or heterocycloalkyl is a N, O or S. In other specific embodiments X5′, X7′, and X11′ are Nle, Leu, Ala, Val, alpha-methylleucine, cycloleucine, tert-leucine, cyclohexylalanine, alpha-methylphenylalanine. Trp, thiazol-5-yl-alanine, 3-(2-pyridyl)-alanine, 3-(3-pyridyl)-alanine, or 3-(4-pyridyl)-alanine. In specific embodiments, X5′, X7′, and X11′ are each independently —(CH₂)p-(C3-C8)alkyl or —(CH₂)p-(C3-C8)hydroxyalkyl wherein p is 0 to 5. In another specific embodiment, X5′, X7′, and X11′ are each independently Nle, Leu, Ala, Val, alpha-methylleucine, cycloleucine, tert-leucine, cyclohexylalanine (e.g., (3-cyclohexyl-L-alanine) or alpha-methylphenylalanine. In a more specific embodiment, X5′ is Nle; and/or X7′ is Leu; and X11′ is Val; and
X6′ and X8′ are each independently absent or are each independently any natural amino acid; or any synthetic amino acid, the side chain of which is H, a —(CH₂)p-(C3-C8)alkyl, —(CH₂)p-(C3-C8)heteroalkyl, a —(CH₂)p-(C3-C8)aryl, —(CH₂)p-(C3-C8)heteroaryl, a —(CH₂)p-(C3-C8)cycloalkyl, or a —(CH₂)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S or a (C1-C6)alkyl, and wherein the heteroatom in the heteroalkyl, heteroaryl or heterocycloalkyl is a N, O or S. In a more specific embodiment, X6′ and/or X8′ are absent.

X13′ is an amino acid, the side chain of which is —(CH₂)p-(C3-C8)aryl, wherein the aryl is optionally substituted with one or more substituents, wherein each substituent is independently an halogen, amine, —OH, S or a (C1-C6)alkyl. In specific embodiments, X14′ is a Phe or an halogen substituted Phe such as a bromophenyl.

In specific embodiments, the compound of Formula (VI) is compound 79 in Table III below.

The present disclosure comprises compounds of Formula (VI), wherein each of X1′ to X14′ are independently defined using any of the more general or more specific definitions provided above for these residues Formula (VI).

In another specific embodiment, the compound comprises or consists in the following formula (VII):

X1′-X2′-X3′-X4′-c[X5′-X6′-X7′-X8′-X9′-X10′]c-X11′-X12′-X13′-X14′-X15′-X16′- X17′,

wherein:
X1′ is absent, —(CH₂)q-CH₃ or —(CF₂)q-CF₃ wherein q is 0 to 11, a natural amino acid, a synthetic amino acid, the side chain of which is H, a —(C1-C12)alkyl, —(CF₂)q-CF₃ wherein q is 0 to 11, —(C3-C8)heteroalkyl, a —(CH₂)p-(C3-C8)aryl, —C(O)—(CH₂)p-(C3-C8)aryl, —(CH₂)p-(C3-C8)heteroaryl, —(CH₂)p-(C3-C8)cycloalkyl, or —(CH₂)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S, —(C1-C6)alkyl, —O—(C1-C6)alkyl, —(CH₂)p-(C3-C8)aryl, wherein p is 0 to 5, —O—(CH₂)p-(C3-C8)aryl, wherein p is 0 to 3, —(C3-C8)cycloalkyl, or —O—(C3-C8)cycloalkyl, and wherein the heteroatom in the heteroalkyl, heteroaryl or heterocycloalkyl is a N, O or S. In a specific embodiment, X1 is Pyr, —(CH₂)q-CH₃ or —(CF₂)q-CF₃ wherein q is 0 to 11. In a more specific embodiment, it is Pyr.

X2′, X3′ and X13′ are each independently a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a specific embodiment, X2′, X3′ and X13′ are each independently an amino acid, the side chain of which is —CH₂—(CH₂)p-guanidine, —CH₂—(CH₂)p-NH₂, or —(CH₂)p-imidazole, preferably —CH₂—(CH₂)p-guanidine, wherein p is 0 to 4, optionally substituted with e.g., an aryl. In a specific embodiment, X2′, X3′ and X13′ are each independently Lys, Orn, Dab (2,4-diaminobutyric acid), Dap (2,3-diaminopropionic acid), Arg, hArg, or His. In a more specific embodiment, X2′, X3′ and X13′ are each independently —CH₂—(CH₂)p-guanidine, wherein p is 0 to 4, optionally substituted with e.g., an aryl. In a more specific embodiment, X2′ and X3′ are each independently Arg, aryl-substituted Arg (e.g., —C(O)—(C3-C8)aryl such as 4bromobenzoyl), hArg, Nle, Leu, Ala, Val, alpha-methylleucine, cycloleucine, tert-leucine, cyclohexylalanine (e.g., (3-cyclohexyl-L-alanine) or alpha-methylphenylalanine. In a more specific embodiment, X2′, and/or X3′ are each independently Arg or hArg (substituted or not (e.g., Arg substituted with 4bromobenzoyl); and X13′ is Arg.

X4′, X6′, X8′ and X12′ are each independently absent or is any natural amino acid, or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S or a (C1-C6)alkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a more specific embodiment, X4′, X6′, X8′ and X12′ are each independently a —(CH₂)p-(C3-C8)alkyl or —(CH₂)p-(C3-C8)hydroxyalkyl wherein p is 0 to 5. In a more specific embodiment, X4′, X6′, X8′ and X12′ are each independently Leu, Nle, alpha-methylleucine, cycloleucine, tert-leucine, cyclohexylalanine (e.g., (3-cyclohexyl-L-alanine), alpha-methylphenylalanine, Ala, Val, Ile, Ser or Thr. In a more specific embodiment, X4′, X6′, X8′ and X12′ are each independently Ser, Nle or Leu. In a more specific embodiment, X4′ and/or X12′ are Ser; and/or X6′ is Nle; and/or X8′ is Leu.

X5′ and X10′ close the ring and are identical or different and are aliphatic residues, alkenyl residues, acid residues or a natural or non-natural amino acid, or a derivative thereof, these moieties being optionally substituted. In specific embodiments, they are residues bearing a terminal alkene or free carboxylic or amine function. In specific embodiments, they are each independently Lys, Orn, Dab (2,4-diaminobutyric acid), Dap (2,3-diaminopropionic acid), Asp, Glu, AllylGly, Nα-allyl-arginine, Nπ-allyl-histidine, Nγ-allyl-Nγ-nosyl-α,γ-diamino-butanoic acid, Nγ-allyl-α,γ-diamino-butanoic acid, or Nγ-allyl-Nγ-methyl-α,γ-diamino-butanoic acid whereby the cycle is closed by an amide bridge or an alkene. In a specific embodiment, there are each independently Lys, Orn, Dab, Dap, Asp, Glu, or AllylGly, wherein the cycle is closed by an amide bridge or an alkene. In specific embodiments, one of X1′ and X4′ is Glu and the other is Lys. In a specific embodiment, they are Lys and Glu or Glu and Lys;

X7′, X15′ and X17′ are each independently Gly, Phe, Leu, Ile, Ser, Pro, Aib, Sar, Oic, βAla, Hyp or Hyp(OBn). In a specific embodiment, X7′, X15′ and X17′ are each Pro.

X9′ and X11′ are each independently absent or a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a specific embodiment, X9′ and X11′ are each independently absent or is an amino acid, the side chain of which is —CH₂—(CH₂)p-guanidine, —CH₂—(CH₂)p-NH₂, or —(CH₂)p-imidazole wherein p is 0 to 4. In a specific embodiment, X9′ is Lys, Orn, Dab (2,4-diaminobutyric acid), Dap (2,3-diaminopropionic acid), Arg, hArg, His or absent. In a more specific embodiment, X9′ and X11′ are each independently absent, are —(CH₂)p-imidazole wherein p is 0 to 4 or are His.

X14′ is any natural amino acid; or any synthetic amino acid, the side chain of which is H, a —(CH₂)p-(C3-C8)alkyl, —(CH₂)p-(C3-C8)heteroalkyl, a —(CH₂)p-(C3-C8)aryl, —(CH₂)p-(C3-C8)heteroaryl, a —(CH₂)p-(C3-C8)cycloalkyl, or a —(CH₂)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S or a (C1-C6)alkyl, and wherein the heteroatom in the heteroalkyl, heteroaryl or heterocycloalkyl is a N, O or S. In another specific embodiment, X14′ is Nle, Leu, Ala, Ile, Val, alpha-methylleucine, cycloleucine, tert-leucine, cyclohexylalanine (e.g., (3-cyclohexyl-L-alanine) or alpha-methylphenylalanine, In specific embodiments, X14′ is a —(CH₂)p-(C3-C8)alkyl wherein p is 0 to 5, or is Ala, Val, Ile, Nle, or Leu; and

X16′ is an amino acid, the side chain of which is —(CH₂)p-(C3-C8)aryl, wherein the aryl is optionally substituted with one or more substituents, wherein each substituent is independently an halogen, amine, —OH, S or a (C1-C6)alkyl. In specific embodiments, X14′ is a Phe or a halogen substituted Phe such as a bromophenyl.

In specific embodiments, the compound of formula (VII) is any one of compounds 74-78 and 89-94 in Table III below. In another specific embodiments, the compound of formula (VII) is any one of compounds 77, 89, 91, and 94 in Table III below.

The present disclosure comprises compounds of Formula (VII), wherein each of X1′ to X16′ are independently defined using any of the more general or more specific definitions provided above for these residues in Formula (VII).In another specific embodiment, the compound comprises or consists in the following formula (VIII):

c[X1′-X2′-X3′-X4′]c-X5′-X6′-X7′-X8′-X9′-X10′-X11′-X12′-X13′-X14′

wherein:
X1′ and X4′ close the ring and are identical or different and are aliphatic residues, alkenyl residues, acid residues or a natural or non-natural amino acid, or a derivative thereof, these moieties being optionally substituted. In specific embodiments, they are residues bearing a terminal alkene or free carboxylic or amine function. In specific embodiments, they are each independently Lys, Orn, Dab (2,4-diaminobutyric acid), Dap (2,3-diaminopropionic acid), Asp, Glu, AllylGly, Nα-allyl-arginine, Nπ-allyl-histidine, Nγ-allyl-NT-nosyl-α,γ-diamino-butanoic acid, Nγ-allyl-α,γ-diamino-butanoic acid, or Nγ-allyl-Nγ-methyl-α,γ-diamino-butanoic acid whereby the cycle is closed by an amide bridge or an alkene. In a specific embodiment, there are each independently Lys, Orn, Dab, Dap, Asp, Glu, or AllylGly. where the cycle is closed by an amide bridge or an alkene In specific embodiments, one of X1′ and X4′ is Glu and the other is Lys. In a specific embodiment, they are Lys and Glu or Glu and Lys;
X2′, X3′, X8′ and X10′ are each independently a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S, preferably N. In a specific embodiment, X2′, X3′, X8′ and X10′ are each independently an amino acid, the side chain of which is —CH₂—(CH₂)p-guanidine, —CH₂—(CH₂)p-NH₂, or —(CH₂)p-imidazole, wherein p is 0 to 4. In a specific embodiment, X2′, X3′, X8′ and X10′ are each independently Lys, Orn, Dab (2,4-diaminobutyric acid), Dap (2,3-diaminopropionic acid), Arg, hArg, or His. In a more specific embodiment, X2′, X3′, X8′ and X10′ are each independently —CH₂—(CH₂)p-guanidine, or —(CH₂)p-imidazole, wherein p is 0 to 4. In another specific embodiment, X2′, X3′ and X10′ are each independently —CH₂—(CH₂)p-guanidine, wherein p is 0 to 4. In another specific embodiment, X2′, X3′ and X10′ are each independently Arg, or hArg; and/or X8′ is His.
X5′, X7′, X9′, and X11′ are each independently a natural amino acid; or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S or a (C1-C6)alkyl; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In another specific embodiment, X5′, X7′, X9′, and X11′ are each independently a —(CH₂)p-(C3-C8)alkyl wherein p is 0 to 5. In another specific embodiment, X5′, X7′, X9′, and X11′ are each independently Leu, Nle, alpha-methylleucine, cycloleucine, tert-leucine, cyclohexylalanine (e.g., (3-cyclohexyl-L-alanine), alpha-methylphenylalanine, Ala, Val, Ile, Ser or Thr. In a more specific embodiment, X5′, X7′, X9′, and X11′ are each independently Ser, Nle, Len or Val. In a more specific embodiment, X5′ is Nle; and/or X7′ is Leu; and/or X9′ is Ser; and/or X11′ is Val.

X6′, X12′ and X14′ are each independently Gly, Phe, Leu, Ile, Ser, Pro, Aib, Sar, Oic, βAla, Hyp or Hyp(OBn), preferably Pro or Oic. In a specific embodiment, X6′, X12′ and X14′ are each Pro.

X13′ is an amino acid, the side chain of which is a —(CH₂)p-(C3-C8)aryl, wherein the aryl is optionally substituted with one or more substituents, wherein each substituent is independently an halogen, amine, —OH, S or a —(C1-C6)alkyl. In another specific embodiment, it is Phe or an halogen substituted Phe. In another embodiment, it is Phe.

In specific embodiments, the compound of formula (VIII) is any one of compounds 72 and 73 in Table III below. In a more specific embodiment, it is compound 72.

In specific embodiments of formula (VI) to (VIII), at least 4 (or at least 5, 6, 7, 8, 9, 10, 11 or 12) of the residues (e.g., at positions Xn defined above, wherein n is 1 to 14 in formula (VI) and (VIII), or 1 to 17 in formula (VII)), other than residues closing the cycle which differ from the corresponding residues in ELA(19-32) (SEQ ID NO: 85), correspond to those in ELA (19-32) (SEQ ID NO: 85). For example, compound 79 has 8 residues corresponding to those in ELA (19-32) (SEQ ID NO: 85).

In other specific embodiments, compounds of the present disclosure correspond to macrocyclic analogs of Ela(19-32) PyrRRCMPLHSRVPFP (SEQ ID NO: 85), wherein the compounds vary from Ela(19-32) by at least two substitutions at positions closing the cycle, and at least one (or 2, 3, 4, 5, 6, 7 or 8) further substitution(s), deletion(s) and/or insertion(s). These substitutions, deletions and/or insertions are defined in the various Xn of formula (VI) to (VIII) above. The correspondence between these Xn and ELA(19-32) is shown in Table C above, wherein the “[” and “]” symbols are used to denote the positions of the ring closure residues in formula (VI) to (VIII) and compounds of the disclosure satisfying these formula.

In formula (I) to (VIII) unless specific otherwise, the link between two amino acid residues (natural or non-natural) are peptide bonds.

In another specific embodiment of formula (I) to (VIII), one of the ring closing residues' is Lys, Dap, Dab, Orn, and the other is Glu or Asp.

In an embodiment, one of the end terminal (natural or non-natural) amino acid residue used for closing the is, before ring-closure, an (natural or non-natural) amino acid having an amine on its lateral chain, and the other end terminal is, before ring-closure, an (natural or non-natural) amino acid having a carboxylic acid on its lateral chain, so that the amine and the carboxylic acid react to form an amide through a macrolactamisation. More specifically, one of the end terminals (natural or non-natural) amino acid residue can be substituted before ring-closure. After closure, the lateral chain carboxylic acid, activated by a coupling agent, has reacted with the amino group of the lateral chain of the other residue to form a peptide bond using, for example, a macrolactamisation reaction.

As used herein, the term “substituted” in reference to above listed natural or unnatural amino acid or acid residues in the structures refers to a substitution by an halogen (e.g., Cl, F, Br, I), —OH, (C1-C6)alkyl, hydroxy(C1-C6)alkyl, (C3-C6)aryl, (C3-C6)aryl(C1-C6)alkyl, (C3-C6)cycloalkyl, hetero(C3-C6)aryl, hetero(C3-C6)aryl(C1-C6)alkyl, hetero(C3-C6)cyclo(C1-C6)alkyl, amino(C1-C6)alkyl, amino(C3-C6)aryl, amino(C3-C6)aryl(C1-C6)alkyl, amino(C3-C6)cycloalkyl, aminohetero(C3-C6)aryl, aminohetero(C3-C6)aryl(C1-C6)alkyl, or amino hetero(C3-C6)cyclo(C1-C6)alkyl.

In specific embodiments, the size of the macrocycle can be of 14 to 24-ring atoms (or 15 to 23, 16 to 22, 17-20). In specific embodiments, the size of the macrocycle can be of 17- to 20-ring atoms.

In all the foregoing compounds, the residues (e.g., X1 to Xn) may be in L or D configurations. In all the foregoing combinations of two residues, they may be in the L, L; L-D; D, L; or D; D configurations.

Without being so limited, specific compounds of the present disclosure encompass those satisfying formula (I) to (VIII). In a more specific embodiment, compounds of the present disclosure encompass compounds listed in Tables I to III.

### Chemical Groups

As used herein, the term “alkyl” refers to a monovalent straight or branched chain, saturated or unsaturated aliphatic hydrocarbon radical having a number of carbon atoms in the specified range. Thus, for example, “(C1-12)alkyl” (or “C1-12 alkyl”) refers to any alkyl of up to 12 carbon atom, including of the hexyl alkyl and pentyl alkyl isomers as well as n-, iso-, sec- and t-butyl, n- and iso- propyl, ethyl, and methyl. As another example, “(C1-4)alkyl” refers to n-, iso-, sec- and t-butyl, n- and isopropyl, ethyl, and methyl. As another example, “C1-3 alkyl” refers to n-propyl, isopropyl, ethyl, and methyl. Alkyl includes unsaturated aliphatic hydrocarbon including alkyne (R—C≡C—R); and/or alkene (R—C═C—R).

The term “halogen” (or “halo”) refers to fluorine, chlorine, bromine and iodine (alternatively referred to as fluoro, chloro, bromo, and iodo). The term “haloalkyl” refers to an alkyl group as defined above in which one or more of the hydrogen atoms have been replaced with a halogen (i.e., F, Cl, Br and/or I). Thus, for example, “C1-10 haloalkyl” (or “C1-C6 haloalkyl”) refers to a C1 to C10 linear or branched alkyl group as defined above with one or more halogen substituents. The term “fluoroalkyl” has an analogous meaning except that the halogen substituents are restricted to fluoro. Suitable fluoroalkyls include the series (CH₂)o-₄CF₃ (i.e., trifluoromethyl, 2,2,2-trifluoroethyl, 3,3,3-trifluoro-n-propyl, etc.).

The term “heteroalkyl” is given its ordinary meaning in the art and refers to alkyl groups as described herein in which one or more carbon atoms is replaced with a heteroatom (e.g., oxygen, nitrogen, sulfur, or derivatives thereof, and the like). Examples of heteroalkyl groups include, but are not limited to, alkoxy, alkyl-substituted amino, thiol such as methionine side group. Up to two heteroatoms may be consecutive. When a prefix such as C₂-6 is used to refer to a heteroalkyl group, the number of carbons (2-6, in this example) is meant to include the heteroatoms as well.

The term “aminoalkyl” refers to an alkyl group as defined above in which one or more of the hydrogen or carbon atoms has been replaced with a nitrogen or an amino derivative such as but not limited to guanidine. Thus, for example, “C₁₋₆ aminoalkyl” (or “C₁-C₆ aminoalkyl”) refers to a C₁ to C₆ linear or branched alkyl group as defined above with one or more amino derivatives (e.g., NH, amide, diazirin, azide, etc.).

The term “thioalkyl” refers to an alkyl group as defined above in which one or more of the hydrogen or carbon atoms has been replaced with a sulfur atom or thiol derivative. Thus, for example, “C₁-6 thioalkyl” (or “C₁-C₆ thioalkyl”) refers to a C₁ to C₆ linear or branched alkyl group as defined above with one or more sulfur atoms or thiol derivatives (e.g., S, SH, etc.).

Aminoalkyl and thioalkyls are specific embodiments of and encompassed by the term “heteroalkyl” or substituted alkyl depending on the heteroatom replaces a carbon atom or an hydrogen atom.

The term “cycloalkyl” refers to saturated alicyclic hydrocarbon consisting of saturated 3-8 membered rings optionally fused with additional (1-3) aliphatic (cycloalkyl) or aromatic ring systems, each additional ring consisting of a 3-8 membered ring. It includes without being so limited cyclopropyl, cyclobutyl, cyclopentyl (cyp) (e.g., compounds 38-41 and 63-65), cyclohexyl and cycloheptane.

The term “heterocyclyl” refers to (i) a 4- to 7-membered saturated heterocyclic ring containing from 1 to 3 heteroatoms independently selected from N, O and S, or (ii) is a heterobicyclic ring (e.g., benzocyclopentyl, octahydroindol (e.g., compound 166)). Examples of 4- to 7-membered, saturated heterocyclic rings within the scope of this disclosure include, for example, azetidinyl, piperidinyl, morpholinyl, thiomorpholinyl, thiazolidinyl, isothiazolidinyl, oxazolidinyl, isoxazolidinyl, pyrrolidinyl, pyridine, imidazolidinyl, piperazinyl, tetrahydrofuranyl, tetrahydrothienyl, pyrazolidinyl, hexahydropyrimidinyl, thiazinanyl, thiazepanyl, azepanyl, diazepanyl, tetrahydropyranyl, tetrahydrothiopyranyl, and dioxanyl. Examples of 4- to 7-membered, unsaturated heterocyclic rings within the scope of this disclosure include mono-unsaturated heterocyclic rings corresponding to the saturated heterocyclic rings listed in the preceding sentence in which a single bond is replaced with a double bond (e.g., a carbon-carbon single bond is replaced with a carbon-carbon double bond).

The term “C(O)” refers to carbonyl. The terms “S(O)₂” and “SO₂” each refer to sulfonyl. The term “S(O)” refers to sulfinyl.

The term “aryl” refers to aromatic (unsaturated) compounds consisting of 3-8 membered rings, optionally fused with additional (1-3) aliphatic (cycloalkyl) or aromatic ring systems, each additional ring consisting of 3-8 membered ring (such as anthracene, indane, Tic, 3-benzothienylalanine, or dihydroindol. In a specific embodiment, it refers to phenyl, benzocyclopentyl, or naphthyl.

The term “heteroaryl” refers to (i) a 3-, 4-, 5- , 6-, 7- or 8-membered heteroaromatic ring (more specifically 3-7 or 3-6 membered ring) containing from 1 to 4 heteroatoms independently selected from N, O and S, such as thiophenyl, thienyl, pyridine, or (ii) is a heterobicyclic ring selected from indolyl, quinolinyl, isoquinolinyl, Tic, dihydroindolylglycine and quinoxalinyl.

Suitable 3-, 4-, 5- and 6-membered heteroaromatic rings include, for example, diazirin, pyridyl (also referred to as pyridinyl), pyrrolyl, diazine (e.g., pyrazinyl, pyrimidinyl, pyridazinyl), triazinyl, thienyl, furanyl, imidazolyl, pyrazolyl, triazolyl (e.g., 1, 2, 3 triazolyl), tetrazolyl (e.g., 1, 2, 3, 4 tetrazolyl), oxazolyl, iso-oxazolyl, oxadiazolyl, oxatriazolyl, thiazolyl, isothiazolyl, and thiadiazolyl. Heteroaryls of particular interest are pyrrolyl, imidazolyl, pyridyl, pyrazinyl, quinolinyl (or quinolyl), isoquinolinyl (or isoquinolyl), and quinoxalinyl. Suitable heterobicyclic rings include indolyl.

The term “aralkyl” and more specifically “(C4-C14)aralkyl” or “C4-14 aralkyl” refers herein to compounds comprising a 3-7 ring-member aryl substituted by a 1 to 7 alkyl. In specific embodiments, it refers to a benzyl or a phenetyl.

As used herein, and unless otherwise specified, the terms “alkyl”, “haloalkyl”, “aminoalkyl”, “cycloalkyl”, “heterocyclyl”, “aryl”, “heteroalkyl” and “heteroaryl” and the terms designating their specific embodiments (e.g., butyl, fluoropropyl, aminobutyl, cyclopropane, morpholine, phenyl, pyrazole, etc.) encompass the substituted (i.e., in the case of haloalkyl and aminoalkyl, in addition to their halogen and nitrogen substituents, respectively) and unsubstituted embodiments of these groups. Hence for example, the term “phenyl” encompasses unsubstituted phenyl as well as fluorophenyl, hydroxyphenyl, methylsulfonyl phenyl (or biphenyl), diphenyl, trifluoromethyl-diazirin-phenyl, isopropyl-phenyl, trifluorohydroxy-phenyl. Similarly, the term pyrazole, encompass unsubstituted pyrazole as well as methylpyrazole. The one or more substituents may be an amine, halogen, hydroxyl, C1-6 aminoalkyl, C1-6 heteroalkyl, C1-6 alkyl, C3-8 cycloalkyl, C1-6 haloalkyl, aryl, heteroaryl and heterocyclyl groups (etc.).

It is understood that the specific rings listed above are not a limitation on the rings which can be used in the present disclosure. These rings are merely representative.

Unless expressly stated to the contrary in a particular context, any of the various cyclic rings and ring systems described herein may be attached to the rest of the compound at any ring atom (i.e., any carbon atom or any heteroatom) provided that a stable compound results therefrom.

Isomers, tautomers and polymorphs

As used herein, the term “isomers” refers to stereoisomers including optical isomers (enantiomers), diastereoisomers as well as the other known types of isomers.

The compounds of the disclosure have at least 5 asymmetric carbon atoms and can therefore exist in the form of optically pure enantiomers (optical isomers), and as mixtures thereof (racemates).

It is to be understood, that, unless otherwise specified, the present disclosure embraces the racemates, the enantiomers and/or the diastereoisomers of the compounds of the disclosure as well as mixtures thereof. Furthermore, certain macrocyclic compounds of the present invention comprise an alkene closing the cycle. Such compounds have Z and E isomers.

For further clarity, (S)—H or (S)—CH₃ indicates that the stereogenic center bearing the H or CH₃ substituent is of (S) stereochemistry.

In addition, the present disclosure embraces all geometric isomers. For example, when a compound of the disclosure incorporates a double bond or a fused ring, both the cis- and trans-forms, as well as mixtures, are embraced within the scope of the disclosure.

Within the present disclosure, it is to be understood that a compound of the disclosure may exhibit the phenomenon of tautomerism and that the formula drawings within this specification can represent only one of the possible tautomeric forms. It is to be understood that the disclosure encompasses any tautomeric form and is not to be limited merely to any one tautomeric form utilized within the formula drawings.

It is also to be understood that certain compounds of the disclosure may exhibit polymorphism, and that the present disclosure encompasses all such forms.

### Salts

The present disclosure relates to the compounds of the disclosure as hereinbefore defined as well as to salts thereof. The term “salt(s)”, as employed herein, denotes basic salts formed with inorganic and/or organic bases. Salts for use in pharmaceutical compositions will be pharmaceutically acceptable salts, but other salts may be useful in the production of the compounds of the disclosure. The term “pharmaceutically acceptable salts” refers to salts of compounds of the present disclosure that are pharmacologically acceptable and substantially non-toxic to the subject to which they are administered. More specifically, these salts retain the biological effectiveness and properties of the anti-atherosclerosis compounds of the disclosure and are formed from suitable non-toxic organic or inorganic acids or bases.

For example, where the compounds of the disclosure are sufficiently acidic, the salts of the disclosure include base salts formed with an inorganic or organic base. Such salts include alkali metal salts such as sodium, lithium, and potassium salts; alkaline earth metal salts such as calcium and magnesium salts; metal salts such as aluminum salts, iron salts, zinc salts, copper salts, nickel salts and a cobalt salts; inorganic amine salts such as ammonium or substituted ammonium salts, such as e.g., trimethylammonium salts; and salts with organic bases (for example, organic amines) such as chloroprocaine salts, dibenzylamine salts, dicyclohexylamine salts, dicyclohexylamines, diethanolamine salts, ethylamine salts (including diethylamine salts and triethylamine salts), ethylenediamine salts, glucosamine salts, guanidine salts, methylamine salts (including dimethylamine salts and trimethylamine salts), morpholine salts, morpholine salts, N,N′-dibenzylethylenediamine salts, N-benzyl-phenethylamine salts, N-methylglucamine salts, phenylglycine alkyl ester salts, piperazine salts, piperidine salts, procaine salts, t-butyl amines salts, tetramethylammonium salts, t-octylamine salts, tris-(2-hydroxyethyl)amine salts, and tris(hydroxymethyl)aminomethane salts. Preferred salts include those formed with sodium, lithium, potassium, calcium and magnesium.

Such salts can be formed routinely by those skilled in the art using standard techniques. Indeed, the chemical modification of a pharmaceutical compound (i.e., drug) into a salt is a technique well known to pharmaceutical chemists, (See, e.g., H. Ansel et. al., Pharmaceutical Dosage Forms and Drug Delivery Systems (6th Ed. 1995) at pp. 196 and 1456-1457, incorporated herein by reference). Salts of the compounds of the disclosure may be formed, for example, by reacting a compound of the disclosure with an amount of acid or base, such as an equivalent amount, in a medium such as one in which the salt precipitates or in an aqueous medium followed by lyophilization.

### Esters

The present disclosure relates to the compounds of the disclosure as hereinbefore defined as well as to the esters thereof. The term “ester(s)”, as employed herein, refers to compounds of the disclosure or salts thereof in which a carboxylic acid has been hydroxy groups have been converted to the corresponding esters using an alcohol and a coupling reagent. Esters for use in pharmaceutical compositions will be pharmaceutically acceptable esters, but other esters may be useful in the production of the compounds of the disclosure.

The term “pharmaceutically acceptable esters” refers to esters of the compounds of the present disclosure that are pharmacologically acceptable and substantially non-toxic to the subject to which they are administered. More specifically, these esters retain the biological effectiveness and properties of the anti-atherosclerosis compounds of the disclosure and act as prodrugs which, when absorbed into the bloodstream of a warm-blooded animal, cleave in such a manner as to produce the parent alcohol compounds.

Esters of the compounds of the present disclosure include among others the following groups (1) carboxylic acid esters obtained by esterification, in which the non-carbonyl moiety of the carboxylic acid portion of the ester grouping is selected from straight or branched chain alkyl (for example, ethyl, n-propyl, t-butyl, n-butyl, methyl, propyl, isopropyl, butyl, isobutyl, or pentyl), n-hexyl, alkoxyalkyl (for example, methoxymethyl, acetoxymethyl, and 2,2-dimethylpropionyloxymethyl), aralkyl (for example, benzyl), aryloxyalkyl (for example, phenoxymethyl), aryl (for example, phenyl optionally substituted with, for example, halogen, C1-4 alkyl, or C1-4 alkoxy, or amino).

Further information concerning examples of and the use of esters for the delivery of pharmaceutical compounds is available in Design of Prodrugs. Bundgaard H ed. (Elsevier, 1985) incorporated herein by reference. See also, H. Ansel et. al., 1995 at pp. 108-109; Krogsgaard-Larsen, 1996 at pp. 152-191; Jarkko Rautio, 2008; and Pen-Wei Hsieh, 2009, all incorporated herein by reference.

The compounds of this disclosure may be esterified by a variety of conventional procedures including the esters are formed from the acid of the molecule by reacting with a coupling agent such as DIC (diisopropyl carbodiimide) and a base, such as NN-dimethylaminopyridine (DMAP), and an alcohol, such as methanol (methyl ester), ethanol, longer chain alcohols or benzyl alcohol (benzyl ester). One skilled in the art would readily know how to successfully carry out these as well as other known methods of esterification of acid.

Esters of the compounds of the disclosure may form salts. Where this is the case, this is achieved by conventional techniques as described above.

### Solvates

The compounds of the disclosure may exist in unsolvated as well as solvated forms with solvents such as water, ethanol, and the like, and it is intended that the disclosure embrace both solvated and unsolvated forms.

“Solvate” means a physical association of a compounds of this disclosure with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Solvates for use in pharmaceutical compositions will be pharmaceutically acceptable solvates, but other solvates may be useful in the production of the compounds of the disclosure.

As used herein, the term “pharmaceutically acceptable solvates” means solvates of compounds of the present disclosure that are pharmacologically acceptable and substantially non-toxic to the subject to which they are administered. More specifically, these solvates retain the biological effectiveness and properties of the anti-atherosclerosis compounds of the disclosure and are formed from suitable non-toxic solvents.

Non-limiting examples of suitable solvates include ethanolates, methanolates, and the like, as well as hydrates, which are solvates wherein the solvent molecules are H₂O.

Preparation of solvates is generally known. Thus, for example, Caira, 2004, incorporated herein by reference, describe the preparation of the solvates of the antifungal fluconazole in ethyl acetate as well as from water. Similar preparations of solvates, hemisolvate, hydrates and the like are described by van Tonder, 2004; Bingham, 2001, both incorporated herein by reference.

A typical, non-limiting, process for preparing a solvate involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example IR spectroscopy, can be used to show the presence of the solvent (or water) in the crystals as a solvate (or hydrate).

### Compositions, Combination and Kits

### Compositions

The present disclosure also relates to pharmaceutical compositions comprising the above-mentioned compounds of the disclosure or their pharmaceutically acceptable salts, esters and solvates thereof and optionally a pharmaceutically acceptable carrier.

As used herein, the terms “pharmaceutically acceptable” refer to molecular entities and compositions that are physiologically tolerable and do not typically produce an allergic or similar untoward reaction, such as gastric upset, dizziness and the like, when administered to subjects (e.g., humans). Preferably, as used herein, the term “pharmaceutically acceptable” means approved by regulatory agency of the federal or state government or listed in the U.S. Pharmacopeia or other generally recognized pharmacopeia for use in animals, and more particularly in humans.

The term “carrier” refers to a diluent, adjuvant, excipient, or vehicle with which the compounds of the present disclosure may be administered. Sterile water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin. The pharmaceutical compositions of the present disclosure may also contain excipients/carriers such as preserving agents, solubilizing agents, stabilizing agents, wetting agents, emulsifiers, sweeteners, colorants, odorants, salts for the variation of osmotic pressure, buffers, coating agents or antioxidants.

In some embodiments, compositions provided herein are administered by one or more routes of administration using one or more of a variety of suitable methods. As will be appreciated by the skilled artisan, the route and/or mode of administration will vary depending upon the desired results. Routes of administration for compounds of the present disclosure for uses disclosed herein include, but are not limited to, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion. Alternatively, compounds of the present disclosure provided herein are administered by a non-parenteral route, such as oral (see e.g., U.S. Pat. No. 7,875,648 B2 to Meier), a topical, epidermal or mucosal route of administration, for example, intranasally, orally, vaginally, rectally, sublingually or topically.

Without being so limited, when the compound/pharmaceutical compositions of the disclosure is administered orally, it may take the form of tablets, coated tablets, dragees, hard or soft gelatin capsules, solutions, emulsions or suspensions for example; rectally using for example of suppositories; locally, topically, or percutaneously, for example using ointments, creams, gels or solutions; or parenterally, e.g., intravenously, intramuscularly, subcutaneously, intrathecally or transdermally, using for example injectable solutions. Furthermore, administration can be carried out sublingually, nasally, or as ophthalmological preparations or an aerosol, for example in the form of a spray, such as a nasal spray.

The compounds of the disclosure may be incorporated into dosage forms in conjunction with any of the vehicles which are commonly employed in pharmaceutical preparations. Methods for preparing appropriate formulations are well known in the art (see e.g., Remington's Pharmaceutical Sciences, 16th Ed., 1980, A. Oslo Ed., Easton, Pa. incorporated herein by reference). Common pharmaceutically acceptable carriers include, without limitation, sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions. Pharmaceutically acceptable carriers also can include physiologically acceptable aqueous vehicles (e.g., physiological saline) or other known carriers appropriate to specific routes of administration.

For the preparation of tablets, coated tablets, dragees or hard gelatin capsules, the compounds of the present disclosure may be admixed with any known pharmaceutically inert, inorganic or organic excipient and/or carrier. Examples of suitable excipients/carriers include lactose, maize starch or derivatives thereof, talc or stearic acid or salts thereof. Suitable excipients for use with soft gelatin capsules include for example vegetable oils, waxes, fats, semi-solid or liquid polyols etc. According to the nature of the active ingredients it may however be the case that no excipient is needed at all for soft gelatin capsules. For the preparation of solutions and syrups, excipients which may be used include for example water, polyols, saccharose, invert sugar and glucose.

For suppositories, and local or percutaneous application, excipients which may be used include for example natural or hardened oils, waxes, fats and semi-solid or liquid polyols.

In cases where parenteral administration is elected as the route of administration, preparations containing the compounds of the disclosure may be provided to patients in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles may include fluid and nutrient replenishers, electrolyte replenishers, such as those based upon Ringer's dextrose, and the like.

The medicaments/pharmaceutical compositions may also contain preserving agents, solubilizing agents, stabilizing agents, wetting agents, emulsifiers, sweeteners, colorants, odorants, salts for the variation of osmotic pressure, buffers, coating agents or antioxidants. They may also contain other therapeutically active agents.

The active compounds, in some embodiments, are prepared with carriers that will protect the compound against rapid release, such as a controlled release formulation, including implants, transdermal patches, and microencapsulated delivery systems. Biodegradable, biocompatible polymers used in some embodiments, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. In some embodiments, therapeutic compositions are administered with medical devices known in the art. For example, in one embodiment, therapeutic compositions provided herein are administered with a needleless hypodermic injection device.

Any amount of a pharmaceutical composition can be administered to a subject. The dosages will depend on many factors including the age and the requirements of the patient and the mode of application. Typically, the amount of the compound of the disclosure contained within a single dose will be an amount that effectively prevent, delay or treat the disease or condition to be treated, delayed or prevented without inducing significant toxicity. Hence a “therapeutically effective amount” or “effective amount” or “therapeutically effective dosage” of a specific compound of the disclosure or composition thereof can result in a reduction of pain and/or body temperature in a subject. Intravenous, or oral administrations are preferred forms of use.

The effective amount of the compounds of the disclosure may also be measured directly. The effective amount may be given daily or weekly or fractions thereof. Typically, a pharmaceutical composition of the disclosure can be administered in an amount from about 0.001 mg up to about 500 mg per kg of body weight per day (e.g., 10 mg, 50 mg, 100 mg, or 250 mg). Dosages may be provided in either a single or multiple dosage regimen. For example, in some embodiments the effective amount may range from about 1 mg to about 25 grams of the composition per day, about 50 mg to about 10 grams of the composition per day, from about 100 mg to about 5 grams of the composition per day, about 1 gram of the composition per day, about 1 mg to about 25 grams of the composition per week, about 50 mg to about 10 grams of the composition per week, about 100 mg to about 5 grams of the composition every other day, and about 1 gram of the composition once a week.

These are simply guidelines since the actual dose must be carefully selected and titrated by the attending physician based upon clinical factors unique to each patient. The optimal daily dose will be determined by methods known in the art and will be influenced by factors such as the age of the patient and other clinically relevant factors. In addition, patients may be taking medications for other diseases or conditions. The other medications may be continued during the time that the pharmaceutical composition of the disclosure is given to the patient, but it is particularly advisable in such cases to begin with low doses to determine if adverse side effects are experienced.

### Combinations

In accordance with another aspect, there is provided a combination of at least one of the compounds described herein with another of the compounds described herein and/or with another drug.

### Kits

In accordance with another aspect of the present disclosure, there is provided a kit comprising the compound defined herein or the above-mentioned composition, and instructions to use same in the prevention or treatment of a cardiovascular disease.

In a specific embodiment of the kit, the kit comprises: (i) at least one of the compounds described herein; (ii) another drug for the prevention or treatment of a cardiovascular disease; (iii) instructions to use same in the prevention or treatment of a cardiovascular disease; or (iv) a combination of at least two of (i) to (iii).

### Methods

The present disclosure also relates to a method of preventing or treating a cardiovascular disease or a symptom thereof in a subject in need thereof comprising administering an effective amount a compound of any one of formula (I) and (II) to the subject.

As used herein the term “cardiovascular disease” refers to, without being so limited, heart failure, pulmonary arterial hypertension, cardiac dysfunction in sepsis, cardiac ischemia, and cerebral ischemia.

As used herein the terms “subject” refers to an animal such as, but not limited to a human or a pet or other animal (e.g., pets such as cats, dogs, horses, etc.; and cattle, fishes, swine, poultry, etc.).

As used herein the terms “subject in need thereof” refer to a subject who would benefit from receiving an effective amount of the compound or composition of the present disclosure. In the context of the method of preventing or treating pain, it refers to a subject experiencing or at risk to experience a cardiovascular disease.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the disclosure and does not pose a limitation on the scope of the disclosure unless otherwise claimed.

No language in the specification should be construed as indicating any non-claimed element as essential to the practice of the disclosure.

Herein, the term “about” has its ordinary meaning. In embodiments, it may mean plus or minus 10% of the numerical value qualified.

Unless otherwise defined, all technical and scientific terms used herein have the same meaning as commonly understood by one of ordinary skill in the art to which this disclosure belongs.

Other objects, advantages and features of the present disclosure will become more apparent upon reading of the following non-restrictive description of specific embodiments thereof, given by way of example only with reference to the accompanying drawings.

The present disclosure is illustrated in further details by the following non-limiting examples.

### Example 1: Material and Method

### Reagents and Instruments

All amino acids, HATU, DEPBT, DIPEA, piperidine, DIAD and triphenylphosphine were purchased from Chem-Impex (Illinois, USA). Wang resin (capacity 0.4-0.7 mmol/g) was ordered from Rapp-polymer (Tuebingen, Germany). The Hoveyda-Grubbs catalyst 2nd generation was obtained from Sigma-Aldrich (Ontario, Canada). Other solvents and reagents were purchased from Fischer Scientific (Ontario, Canada) and used as received.

Thin-layer chromatography (TLC) was performed on glass plates precoated with silica gel 60F254 (Merck, Darmstadt, Germany) and visualized with UV light (254 nm) and KMnO₄ spray. Purification of organic molecules was carried out by flash chromatography using a Biotage Isolera One system (Charlotte, North Carolina, US). High-resolution electrospray mass spectroscopy (HRMS) data were recorded with maXis ESI-Q-Tof apparatus (Billerica, USA). Analytical LC was performed using UPLC-MS system from Waters (Milford, USA) (column Acquity UPLC® CSH™ C18 (2.1×50 mm) packed with 1.7 μm particles). ¹H and ¹³C NMR spectra (298 K) were recorded at 400 MHz and 100 MHz respectively on Bruker Ascend 400 or at 600 MHz on a Bruker 600 MHz Varian INOVA spectrometer. Chemical shifts are in parts per million (ppm). Residual solvent signals were used as internal standard.

### Fmoc-Based Amino Acid Synthesis

Synthesis of N^{α}-Fmoc-(N^{π}-allyl)-L-histidine-OH.

Methyl N^{α}-(((9H-fluoren-9-yl)methoxy)carbonyl)-N^{π}-trityl-L-histidinate (Fmoc-L-His(Trt)—OMe 103). To a suspension of Fmoc-L-His(Trt)—OH (1 equiv., 3.1 g, 5 mmol) and 1-hydroxy-benzotriazol monohydrate (HOBt·H₂O, 1.1 equiv., 1.05 g, 5.5 mmol) in 30 mL DCM, was added 1-ethyl-2-diaminopropyl-carbodiimine hydrochloride (EDC- HCl, 1.1 equiv., 743 mg, 5.5 mmol). A small amount of MgSO4 (-100 mg) was added to absorb water and the mixture was stirred for 10 min at room temperature. Methanol (1 mL) was added and the reaction was run overnight at rt. The organic phase was washed twice with water, once with brine, dried with MgSO4 and filtered. Organic solvent was removed under vacuum and the product was purified by flash chromatography using DCM-MeOH (95:5) as eluents. The product was obtained as a white solid, 2.33 g, yield 73%. The characterization of product is identical to that reported (Ahn et al., 2013).

Methyl N^{α}-(((9H-fluoren-9-yl)methoxy)carbonyl)-N^{π}-allyl-L-histidinate (N^{α}-Fmoc-(N^{π}-allyl)-L-histidine-OMe 104). To a solution of freshly distilled Tf₂O (1.1 equiv., 686 μL, 4.0 mmol) in 26 mL DCM pre-cooled at −78° C., was added a mixture of allylic alcohol (1.1 equiv., 278 μL, 4.0 mmol) and diisopropylethylamine (DIPEA, 1.2 equiv., 855 μL, 4.4 mmol) in 18 mL DCM (dropwise for 15 min). The reaction mixture was stirred at −78° C. for 30 min and transferred slowly into a solution of Fmoc-His(Trt)—OMe (1 equiv., 2.32 g, 3.7 mmol) dissolved in 30 mL DCM pre-cooled at −78° C.). After 10 min, the reaction mixture was warmed up to room temperature and stirred overnight at rt. The residual acid was neutralized by mixing vigorously with 10 mL saturated NaHCO₃. The organic phase was washed twice with saturated NaHCO₃, dried with MgSO4, filtered and evaporated to dryness. The trityl group was cleaved by treating the crude with a solution of TFA/TIPS (2 mL/0.5 mL) in 20 mL DCM for 2 h at rt. The mixture was evaporated to dryness, the residual acid was neutralized with 40 mL saturated NaHCO₃ and the product was extracted with EtOAc. The purification was carried out using flash chromatography with DCM-MeOH (gradient 0-410% MeOH during 10 CV). Product was obtained as a white foam, 1.1 g, yield 70%. ¹H NMR (400 MHz, CDCl₃) δ (ppm) 8.58 (s, 1H), 7.74 (d, J=7.5 Hz, 2H), 7.55 (d, J=7.3 Hz, 2H), 7.38 (t, J=7.4 Hz, 2H), 7.29 (t, J=7.4 Hz, 2H), 7.18 (s, 1H), 6.09 (d, J=7.2 Hz, 1H, proton amide), 5.96-5.76 (m, 1H), 5.36 (d, J=10.2 Hz, 1H), 5.16 (d, J=17.0 Hz, 1H), 4.76-4.60 (m, 2H), 4.59-4.49 (m, 1H), 4.37 (d, J=5.5 Hz, 2H), 4.16 (t, J=6.5 Hz, 1H), 3.75 (s, 3H), 3.28-3.05 (m, 2H). 13C NMR (101 MHz, CDCl₃) δ (ppm) 170.64, 156.16, 143.66, 143.59, 141.41, 135.31, 129.86, 129.78, 127.95, 127.25, 125.09, 121.37, 120.15, 119.42, 67.27, 53.21, 52.85, 49.13, 47.09, 26.35. HRMS [M+Na+]: 454.1750 (calculated 454.1737).

N^{α}-(((9H-fluoren-9-yl)methoxy)carbonyl)-N^{π}-allyl-L-histidine (N^{α}—Fmoc-(N^{π}-allyl)-L-histidine-OH 105). To a solution of Nα-Fmoc-(Nπ-allyl)-L-histidine-OMe (602 mg, 1.4 mmol) in 27 mL dioxane, 27 mL HCl 2 M was added and the mixture was refluxed for 10 h. The solution was neutralized to pH 6 using NaOH 1 M. This mixture was extracted twice with DCM. TLC was used to monitor the completion of the extraction process. The combined organic phases were washed with brine, dried over MgSO4 and filtered. Organic solvents were removed under vacuum and the crude product was purified by flash chromatography using DCM-MeOH as eluents (gradient 5% →20% MeOH+1% AcOH during 10 CV). Obtained 450 mg of product, yield 77%. ¹H NMR (400 MHz, MeOD-d4) δ(ppm) 8.91 (s, 1H), 7.78 (d, J=7.5 Hz, 2H), 7.62 (t, J=6.8 Hz, 2H), 7.38 (t, J=7.4 Hz, 2H), 7.35-7.25 (m, 3H), 6.04 (ddd, J=22.4, 10.8, 5.6 Hz, 1H), 5.40 (d, J=10.3 Hz, 1H), 5.24 (d, J=17.1 Hz, 1H), 4.87 (d, J=4.5 Hz, 2H), 4.53 (dd, J=9.4, 4.6 Hz, 1H), 4.37 (d, J=6.7 Hz, 2H), 4.18 (t, J=6.6 Hz, 1H), 3.38-3.30 (m, 1H), 3.10 (dd, J=15.9, 9.6 Hz, 1H). ¹³C NMR (101 MHz, MeOD-d4) δ(ppm) 173.27, 158.40, 145.16, 145.07, 142.60, 136.54, 132.85, 132.08, 128.83, 128.16, 126.12, 120.96, 119.36, 67.88, 53.63, 50.24, 48.36, 26.82. HRMS [M+Na⁺]: 418.1759 (calculated 418.1759).

### Synthesis and Characterization of Tyr(OBn) Analogs

(S)-2-amino-3-(3-cyclopentyl-4-hydroxyphenyl)-propanoic (cypTyr 107). To a 250 mL round bottom flask containing L-Tyrosine (1 equiv., 4.0 g, 22 mmol), was added H₃PO₄ 85% (7.8 equiv., 20 mL, 173 mmol) and 3 mL cyclopentanol (1.5 equiv., 2.9 g, 33 mmol) and the suspension was stirred and heated at 100° C. overnight. After 16 h, the reaction mixture was cooled down, diluted in 100 mL ice water and the acid was neutralized with KOH (15.6 equiv., 19.3 g, 345 mmol). NaHCO₃ was added until pH 5-7 to precipitate the product. The precipitate was filtered and washed with cold water. The solid was dried under the fume hood for 1 day, to deliver 4.96 g crude product (off-white solid) as a mixture of mono- and dialkylated tyrosine which was used as such for the next step.

Methyl (S)-2-amino-3-(3-cyclopentyl-4-hydroxyphenyl)-propanoate (cypTyr-OMe, 108). The crude mixture of compound 107 (4.0 g) was dissolved in anhydrous methanol (73 equiv., 45 mL, 1.1 mol) in a 250 mL round-bottom flask under inert atmosphere. The solution was cooled down to 0° C. then thionyl chloride (3 equiv., 3.3 mL, 46 mmol) was added slowly to the flask. The mixture was stirred overnight at room temperature. After 15 h, methanol was evaporated under reduced pressure and the mixture was diluted with cold saturated NaHCO₃ (15 mL). The products were extracted with three portions of ethyl acetate (EtOAc, 20 mL each). The combined organic phases were washed with one portion of brine (20 mL), dried over anhydrous MgSO₄ and filtered. The EtOAc was evaporated under reduce pressure to obtain 3.85 g brown-orange oil. The obtained product was purified using flash chromatography with a gradient elution 0→7% MeOH in DCM on a normal phase silica gel cartridge. The mono- and dialkylated products were entirely separated and the monoalkylated product 15 was obtained as a clear, sticky oil (1.2 g), yield 26% (2 steps). ¹H NMR (400 MHz, MeOD-d4) δ(ppm) 6.93 (d, J=2.0 Hz, 1H), 6.79 (dd, J₁=8.0, 2.0 Hz, 1H), 6.67 (d, J=8.4 Hz, 1H), 3.67 (s, 3H), 3.64 (t, J=6.4 Hz, 1H), 3.32-3.22 (m, 1H), 2.92-2.81 (m, 2H), 2.03-1.94 (m, 2H), 1.86-1.50 (m, 6H). ¹³C NMR (101 MHz, MeOD-d4) δ(ppm) 176.6, 155.4, 133.8, 128.9, 128.6, 128.4, 116.1, 56.9, 52.5, 41.2, 40.6, 34.2, 34.1, 26.6.

Methyl (S)-2-((tert-butoxycarbonyl)amino)-3-(3-cyclopentyl-4-hydroxyphenyl)-propanoate (Boc-cypTyr-OMe 109). Compound 108 (1 equiv., 1.2 g, 0.7 mmol) was dissolved in a 30 mL mixture of THF/H₂O (1:1) in a 250 mL round-bottom flask. NaHCO₃ (2 equiv., 114 mg, 1.4 mmol) was added to the flask and stirred until complete dissolution. Di-tert-butyl-dicarbonate (1.5 equiv., 223 mg, 1.0 mmol) was added and the reaction was run for 1 h at room temperature. To neutralize the solution, aqueous HCl 1 M (1.4 mL, 1.4 mmol) was added and THF was evaporated in vacuo. The final product was extracted from the aqueous phase with three portions of EtOAc (20 mL each). The combined organic phases were washed with one portion of brine (20 mL) and dried with anhydrous magnesium sulfate. The solution was filtered and the solvent was removed in vacuo. The product was purified by flash chromatography using a gradient of 20→30% EtOAc in hexane to provide 16 as a pale-yellow oil (1.64 g), yield 84%. ¹H NMR (400 MHz, CDCl₃) δ (ppm) 6.90 (d, J=2.0 Hz, 1H), 6.77 (dd, J=8.1, 2.2 Hz, 1H), 6.64 (d, J=8.0 Hz, 1H), 4.99 (d, J=8.2 Hz, 1H), 4.54 (dd, J=13.7, 5.7 Hz, 1H), 3.71 (s, 3H), 3.28-3.14 (m, 1H), 3.00 (d, J=5.2 Hz, 2H), 2.10-1.93 (m, 2H), 1.86-1.73 (m, 2H), 1.73-1.63 (m, 2H), 1.61-1.51 (m, 2H), 1.42 (s, 9H). ¹³C NMR (101 MHz, CDCl₃) δ (ppm) 172.76, 155.33, 152.92, 132.27, 128.07, 127.56, 127.38, 115.41, 80.14, 54.64, 52.35, 38.94, 37.74, 33.07, 33.02, 28.43, 25.49.

Methyl (S)-3-(4-(benzyloxy)-3-cyclopentylphenyl)-2-((tert-butoxycarbonyl)amino)-propanoate (Boc-cypTyr(OBn)—OMe 110). Compound 109 (1 equiv., 203 mg, 559 μmol) was dissolved in dry acetonitrile (5 mL) and K₂CO₃ (1.2 equiv., 92.6 mg, 670 μmol) was added to the mixture, followed by benzyl bromide (1.3 equiv., 130 mg, 760 μmol). The mixture was refluxed for 18 h. After the reaction was complete, acetonitrile was evaporated in vacuo. The crude was redissolved in 15 mL EtOAc, washed with 3 portions of water (15 mL each) and 20 mL brine. The organic phase was collected, dried with MgSO₄, filtered and the solvent was removed in vacuo. The crude was purified by flash chromatography using hexane-EtOAc (8:2) as isocratic eluent to obtain a clear sticky oil (158 mg), yield 58%. ¹H NMR (400 MHz, CDCl₃) δ (ppm) 7.42-7.24 (m, 5H), 6.95 (d, J=1.8 Hz, 1H), 6.86 (dd, J=7.8, 1.5 Hz, 1H), 6.80 (d, J=8.4 Hz, 1H), 5.04 (s, 2H), 4.95 (d, J=8.1 Hz, 1H), 4.54 (q, J=7.7 Hz, 1H), 3.70 (s, 3H), 3.38 (quint, J=8.4 Hz, 1H), 3.02 (m, 2H), 2.01 (m, 2H), 1.79-1.50 (m, 6H), 1.42 (s, 9H). ¹³C NMR (101 MHz, CDCl₃) δ (ppm) 172.7, 155.8, 155.3, 137.7, 135.3, 128.7, 128.1, 128.0, 127.9, 127.8, 127.3, 111.9, 80.0, 70.3, 54.7, 52.4, 39.1, 37.8, 33.2, 28.5, 25.7.

Methyl (S)-2-((tert-butoxycarbonyl)amino)-3-(3-cyclopentyl-4-(cyclopentyloxy)phenyl)-propanoate (Boc-cypTyr(OCyp)—OMe 111). From 109 (1.00 g, 2.77 mmol), used similar protocol as synthesis of 110 to provide 111 (640 mg, 1.48 mmol, yield 54%). ¹H NMR (400 MHz, CDCl₃) δ (ppm) 6.89 (s, 1H), 6.83 (d, J=7.6 Hz, 1H), 6.71 (d, J=8.0 Hz, 1H), 5.05-4.84 (m, 1H), 4.82-4.64 (m, 1H), 4.60-4.41 (m, 1H), 3.69 (s, 3H), 3.21 (quint, J=7.6 Hz, 1H), 2.99 (d, J=4.4 Hz, 2H) 2.08-1.28 (m, 25H). ¹³C NMR (101 MHz, CDCl₃) δ (ppm) 172.7, 155.2, 155.0, 135.3, 128.1, 127.1, 126.9, 112.4, 79.9, 79.1, 54.6, 52.3, 39.5, 37.7, 33.0, 32.9, 28.5, 25.7, 24.2.

Methyl (S)-2-((tert-butoxycarbonyl)amino)-3-(3-cyclopentyl-4-propoxyphenyl)-propanoate (Boc-cypTyr(OPr)—OMe 112). From 109 (900 mg, 2.48 mmol), used similar protocol as synthesis of 110 to obtain 112 (521 mg), yield 52%. 1H NMR (600 MHz, CDCl₃) δ (ppm) 6.90 (d, J=1.2 Hz, 1H), 6.84 (dd, J=8.4 Hz, 1.8 Hz, 1H), 6.71 (d, J=8.4 Hz, 1H), 4.92 (d, J=7.8 Hz, 1H), 4.51 (q, J=7.8 Hz, 1H), 3.87 (t, J=5.4 Hz, 2H), 3.69 (s, 3H), 3.28 (quint, J=8.4 Hz, 1H), 2.99 (d, J=6.6 Hz, 2H), 2.01-1.93 (m, 2H), 1.79 (sext, J=6.0 Hz, 2H) 1.76-1.70 (m, 2H), 1.68-1.48 (m, 4H), 1.40 (s, 9H), 1.02 (t, J=7.2 Hz, 3H). ¹³C NMR (101 MHz, CDCl₃) δ (ppm) 172.7, 156.2, 155.3, 135.0, 128.0, 127.4, 127.3, 111.4, 80.0, 69.8, 54.7, 52.4, 39.3, 37.8, 33.1, 28.5, 25.8, 23.0, 11.0.

(S)-3-(4-(benzyloxy)-3-cyclopentylphenyl)-2-((tert-butoxycarbonyl)amino)-propanoic (Boc-cypTyr(OBn)—OH 113). To a solution of compound 110 (921 mg, 2.03 mmol) in 10 mL THF, was added LiOH (583 mg, 24.4 mmol, pre-dissolved in 10 mL water). The mixture was stirred at room temperature for 3 h. Upon completed conversion (as followed by TLC), pH was adjusted to 5 with HCl 1 M and THF was removed in vacuo. The pH of aqueous phase was adjusted to 2 and the product was extracted with three portions of ethyl acetate (20 mL each). The combined organic phase was dried with MgSO4, filtered and the solvent was removed in vacuo to obtain a white solid (860 mg), yield 96%. The product is pure enough to continue to the next step without purification. ¹H NMR (400 MHz, CDCl₃), δ(ppm) 7.46-7.36 (m, 4H), 7.36-7.29 (m, 1H), 6.95 (d, J=7.8 Hz, 1H), 6.83 (d, J=8.3 Hz, 1H), 5.06 (s, 2H), 4.95 (d, J=7.9 Hz, 1H), 4.60 (dd, J=12.4, 5.6 Hz, 1H), 3.50-3.31 (m, 1H), 3.22-2.96 (m, 2H), 2.09-1.95 (m, 2H), 1.84-1.72 (m, 2H), 1.71-1.55 (m, 4H), 1.49-1.28 (m, 10H). ¹³C NMR (101 MHz, CDCl₃) δ (ppm) 177.05, 155.80, 155.46, 137.61, 135.32, 128.63, 128.14, 127.84, 127.77, 127.34, 127.26, 111.92, 80.29, 77.48, 77.16, 76.84, 70.23, 54.46, 39.05, 37.23, 33.10, 28.42, 25.61.

(S)-2-((tert-butoxycarbonyl)amino)-3-(3-cyclopentyl-4-(cyclopentyloxy)phenyl)-propanoic acid (Boc-cypTyr(OCyp)—OH 114). From 111 (640 mg, 1.48 mmol), used the same protocol as synthesis of 113 to provide 114 (656 mg, 1.57 mmol, 100%). ¹H NMR (400 MHz, CDCl₃) δ (ppm) 6.98 (s, 1H), 6.92 (d, J=7.9 Hz, 1H), 6.75 (d, J=8.4 Hz, 1H), 4.91 (d, J=7.9 Hz, 1H), 4.74 (p, J=4.0 Hz, 1H), 4.56 (d, J=6.6 Hz, 1H), 3.32-3.17 (m, 1H), 3.16-2.91 (m, 2H), 1.95 (dd, J=10.3, 5.3 Hz, 2H), 1.90-1.82 (m, 4H), 1.81-1.71 (m, 4H), 1.70-1.59 (m, 4H), 1.59-1.50 (m, 2H), 1.48-1.28 (m, 9H). ¹³C NMR (101 MHz, CDCl₃) δ (ppm) 177.03, 155.52, 155.03, 135.42, 128.20, 127.13, 126.71, 112.48, 80.29, 79.11, 54.49, 39.56, 37.16, 33.03, 32.85, 28.42, 25.71, 24.16.

(S)-2-((tert-butoxycarbonyl)amino)-3-(3-cyclopentyl-4-propoxyphenyl)-propanoic acid (Boc-cypTyr(OPr)—OH 115). From 112 (521 mg, 1.28 mmol), used the same protocol as synthesis of 113 to obtain 115 (467 mg, 1.19 mmol, yield 93%). ¹H NMR (400 MHz, CDCl₃) δ (ppm) 6.97 (s, 1H), 6.91 (d, J=8.0 Hz, 1H), 6.72 (d, J=8 Hz, 1H), 4.94-4.79 (m, 1H), 4.57-4.45 (m,1H), 3.88 (t, J=6 Hz, 2H), 3.29 (quint, J=8 Hz, 1H), 3.13-2.91 (m, 2H), 2.05-1.89 (m, 2H), 1.85-1.46 (m, 8H), 1.40 (s, 9H), 1.02 (t, J=7.6 Hz, 3H). ¹³C NMR (101 MHz, CDCl₃), δ(ppm) 177.0, 156.3, 155.6, 135.1, 128.2, 127.4, 127.2, 111.5, 80.4, 69.8, 54.6, 39.4, 37.3, 33.1, 28.5, 25.8, 23.0, 11.0.

(S)-2-((((9H-fluoren-9-yl)methoxy)carbonyl)amino)-3-(4-(benzyloxy)-3-cyclopentylphenyl)-propanoic acid (Fmoc-cypTyr(OBn)—OH 116). Compound 113 (1 equiv., 860 mg, 1.96 mmol) was dissolved in 20 mL mixture TFA-DCM (1:1) and stirred for 2 h at rt. TFA and DCM were evaporated in vacuo to provide a green-brown solid. The residual acid was neutralized with saturated NaHCO₃ solution and 20 mL THF was added to solubilize the solid residue. NaHCO₃ (3 equiv., 493 mg, 5.87 mmol) was dissolved in 10 mL water and added to the mixture, followed by Fmoc-Cl (1.1 equiv., 607 mg, 2.07 mmol). The reaction was run for 2 h at rt. THF was evaporated in vacuo and the aqueous phase was acidified with aqueous HCl 1 N until pH 3, extracted with 3 portions of EtOAc (15 mL). The combined organic phases were washed with brine and dried with MgSO₄. Organics were evaporated in vacuo to deliver the crude product.

The product was purified by flash chromatography using a gradient 0-30% EtOAc+0.25% AcOH in hexanes to provide a pale-yellow oil (752 mg), yield (68%). ¹H NMR (400 MHz, CDCl₃), δ(ppm) 7.76 (d, J=7.5 Hz, 2H), 7.55 (t, J=7.9 Hz, 2H), 7.46-7.27 (m, 10H), 7.06 (s, 1H), 6.91 (d, J=8.2 Hz, 1H), 6.81 (d, J=8.3 Hz, 1H), 5.20 (d, J=8.2 Hz, 1H), 5.03 (s, 2H), 4.69 (dd, J=13.5, 5.8 Hz, 1H), 4.46-4.32 (m, 2H), 4.20 (t, J=7.0 Hz, 1H), 3.45-3.31 (m, 1H), 3.17 (dd, J=14.1, 5.3 Hz, 1H), 3.09 (dd, J=14.0, 6.1 Hz, 1H), 2.01 (s, 2H), 1.82-1.69 (m, 2H), 1.68-1.51 (m, 5H). ¹³C NMR (101 MHz, CDCl₃), δ(ppm) 176.67, 155.94, 143.89, 143.85, 141.42, 137.53, 135.49, 128.65, 127.99, 127.88, 127.42, 127.39, 127.29, 127.24, 127.20, 125.23, 120.13, 111.99, 70.22, 67.33, 54.81, 47.24, 39.13, 37.27, 33.13, 25.62, 25.60. HRMS [M+Na+]584.2405 (calculated 584.2407).

(S)-2-((((9H-fluoren-9-yl)methoxy)carbonyl)amino)-3-(3-cyclopentyl-4-(cyclopentyloxy) phenyl)-propanoic acid (Fmoc-cypTyr(OCyp)—OH 117). From 114 (600 mg, 1.44 mmol), used similar protocol as synthesis of 116 to provide 117 (321 mg, 593 μmol, yield 41%). ¹H NMR (400 MHz, CDCl₃) δ (ppm) 7.76 (d, J=7.5 Hz, 2H), 7.56 (t, J=7.9 Hz, 2H), 7.40 (t, J=7.4 Hz, 2H), 7.30 (t, J=7.4 Hz, 2H), 7.01 (s, 1H), 6.90 (d, J=8.1 Hz, 1H), 6.74 (d, J=8.3 Hz, 1H), 5.22 (d, J=8.2 Hz, 1H), 4.79-4.64 (m, 2H), 4.38 (t, J=6.9 Hz, 2H), 4.21 (t, J=7.0 Hz, 1H), 3.30-2.93 (m, 3H), 2.03-1.91 (m, 2H), 1.85 (d, J=4.6 Hz, 4H), 1.82-1.71 (m, 4H), 1.70-1.59 (m, 4H), 1.60-1.48 (m, 2H). ¹³C NMR (101 MHz, CDCl₃) δ (ppm) 176.79, 155.97, 155.15, 143.91, 141.41, 135.54, 128.06, 127.84, 127.19, 126.40, 125.25, 120.10, 112.52, 79.11, 67.33, 54.82, 47.24, 39.63, 37.23, 33.02, 32.86, 25.71, 25.68, 24.17. HRMS [M+H⁺] 540.2762 (calculated 540.2745).

(S)-2-((((9H-fluoren-9-yl)methoxy)carbonyl)amino)-3-(3-cyclopentyl-4-propoxyphenyl)-propanoic acid (Fmoc-cypTyr(OPr)—OH 118). From 115 (467 mg, 1.19 mmol), used similar protocol as synthesis of 116 to provide 118 (438 mg, 853 μmol, yield 71%). ¹H NMR (400 MHz, CDCl₃) δ (ppm) 7.76 (d, J=7.6 Hz, 2H), 7.55 (t, J=7.8 Hz, 2H), 7.40 (t, J=7.4 Hz, 2H), 7.30 (t, J=7.4 Hz, 2H), 7.02 (s, 1H), 6.91 (d, J=8.2 Hz, 1H), 6.73 (d, J=8.3 Hz, 1H), 5.19 (d, J=8.2 Hz, 1H), 4.68 (dd, J=13.6, 5.8 Hz, 1H), 4.43-4.33 (m, 2H), 4.20 (t, J=7.0 Hz, 1H), 3.88 (t, J=6.3 Hz, 2H), 3.39-3.24 (m, 1H), 3.16 (dd, J=14.1, 5.3 Hz, 1H), 3.08 (dd, J=14.0, 6.2 Hz, 1H), 2.09-1.90 (m, 2H), 1.87-1.71 (m, 4H), 1.70-1.61 (m, 2H), 1.60-1.50 (m, 2H), 1.04 (t, J=7.4 Hz, 3H). ¹³C NMR (101 MHz, CDCl₃) δ (ppm) 176.65, 156.32, 155.97, 143.90, 141.41, 135.11, 127.91, 127.85, 127.34, 127.20, 126.78, 125.24, 120.11, 111.42, 69.65, 67.34, 54.83, 47.24, 39.33, 37.26, 33.02, 25.66, 25.63, 22.89, 10.94. HRMS [M+H⁺] 514.2602 (calculated 514.2588).

### General Protocol for Solid Phase Synthesis

Subject to more specific protocols provided in Examples 2-4, the following provides a general protocol for solid phase synthesis of analogs disclosed herein.

Peptides were synthesized on solid phase at 0.1 mmol scale using Fmoc-based chemistry. The first amino acid was loaded into the resin using Mitsunobu reaction. In short, amino acid (0.3 equiv., 0.3 mmol), triphenylphosphine (3 equiv., 0.3 mmol, 79 mg) and 300 mg Wang resin were mixed together in 4 mL DCM for 5 min. Diisopropyl azodicarboxylate (DIAD, 3 equiv., 0.3 mmol, 59 μL) was added dropwise and the mixture was shaken overnight. Excess reagents were removed by washing twice with 5 mL DCM. The amino acid loading was quantified by measuring UV absorbance of dibenzofulvene-piperidine adduct resulting from Fmoc deprotection. The loading was usually 0.25-0.35 mmol/g. When the desired loading was achieved, the resin was capped with 4 mL of a solution of DCM-acetic anhydride-DIPEA (4:1:0.2) during 1 h. The resin was washed with DMF-DCM-iPrOH-DCM-iPrOH-DCM, 3 min with 5 mL of each solvent (aka the washing protocol). The next amino acids were added to sequence by 2 steps: 1/Fmoc deprotection and 2/amide coupling. Resin was always washed using the aforementioned washing protocol between the two steps. Fmoc deprotection was achieved by treating the resin with 5 mL of piperidine 20%/DMF for 10 min. For the coupling steps, HATU (5 eq., 0.5 mmol, 190 mg) and the amino acid (5 equiv, 0.5 mmol) were dissolved in 5 mL DMF, transferred to the resin, then DIPEA (5 equiv., 0.5 mmol, 87 μL) was added to start the coupling reaction. The reaction was run for 30 min and the excess reagents were removed by filtration. The deprotection and coupling steps were repeated to synthesize the linear precursor peptide. The resin was washed using the washing protocol, washed again with diethyl ether and dried overnight in vacuo prior to the cyclization step.

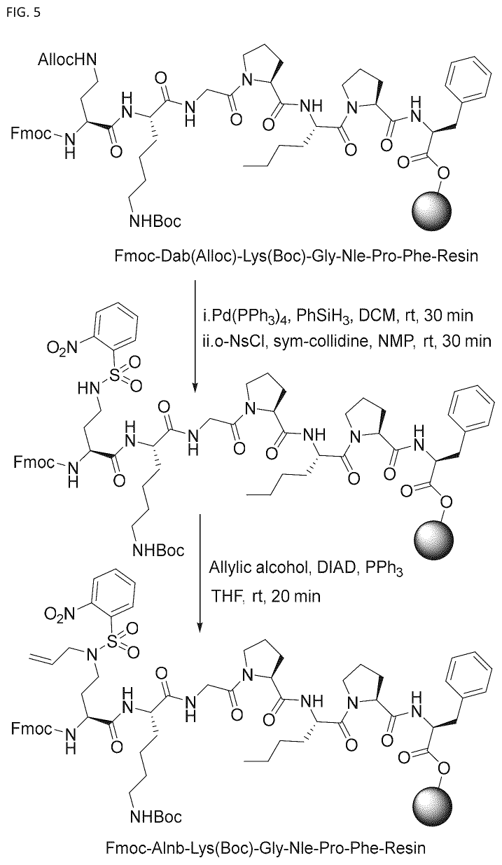

Synthesis of Fmoc-N-allyl-N-nosyl-a, y-diamino-butanoic acid (Fmoc-Alnb-OH) on the resin. The Fmoc-L-Dab(Alloc)—OH was introduced at the His7 position by SPPS and served as the starting residue for synthesis of Fmoc-Alnb-containing peptide (FIG. 5). The Alloc group was removed by treating the resin with a solution of Pd(PPh₃)₄(0.25 equiv., 29 mg, 0.025 mmol), phenylsilane (25 equiv, 311 μL, 2.5 mmol) in DCM under inert atmosphere for 30 min. The resin was washed with 5 mL DCM then 5 mL DMF, 5 min each. In a 20 mL vial, o-nosyl chloride (NsCl, 86 mg, 0.4 mmol) was dissolved in 5 mL of NMP and sym-collidine (55 μL, 0.4 mmol) was added. This mixture was transferred on the resin and agitated for 30 min at rt. The resin was filtered and the nosylation reaction was repeated once. The resin was washed with the washing sequence and dried in vacuo prior to the allylation step. The allylation step was carried out by Fukuyama-Mitsunobu reaction. To a 12 mL cartridge, well-dried resin was swelled with 4 mL anhydrous THE for 5 min and filtered. A mixture of allylic alcohol (68 μL, 1 mmol), PPh₃ (106 mg, 0.4 mmol) in 3 mL of anhydrous THF was poured on the resin and mixed for 5 min prior to adding DIAD dropwise (0.079 mL, 0.4 mmol, diluted in 1 mL anhydrous THF. The mixture was agitated for 20 min at rt. The resin was filtered and the allylation reaction was repeated once. The conversion was monitored by UPLC-MS. The resin was washed with the washing protocol before peptide elongation by SPPS.

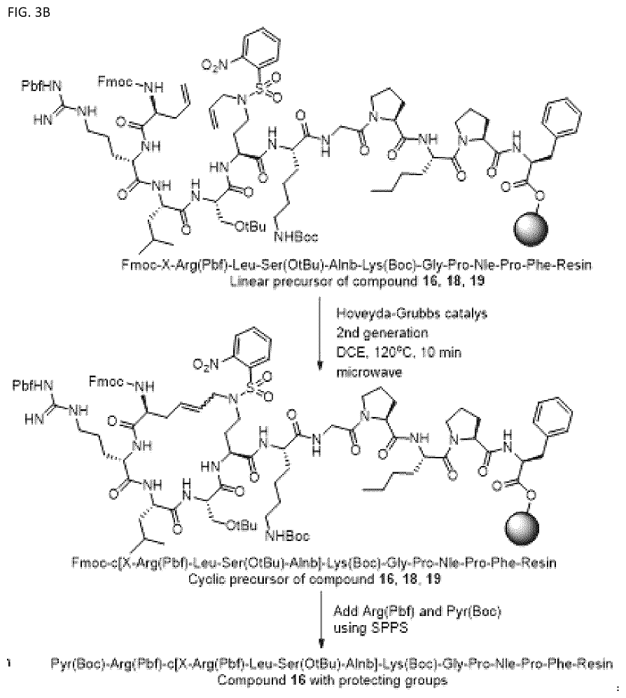

Macrocyclization by ring-closing metathesis. Prior to metathesis, Fmoc-L-allylglycine-OH or Boc-L-Allylglycine (for truncated analogs) was incorporated at the Pro3 position whereas an allyl-containing residue AA (L-allylglycine, N^{π}-allyl-L-histidine or N^{γ}-allyl-N^{γ}-nosyl-α,γ-L-diaminobutyric acid) was introduced at the His7 position during solid phase peptide synthesis (FIG. 3A). The general sequence should be Fmoc/Boc-Allylglycine-Arg(Pbf)-Leu-Ser(OtBu)-AA-Lys(Boc)-Gly-Pro-(C-terminus)-Resin. The dried resin (0.1 mmol peptide) and Hoveyda-Grubbs catalyst 2nd generation (0.023 mmol, 15 mg) were added into a dried 10-mL microwave tube. The teflon cap was added and the tube was filled with argon through a needle by vacuum and backfill cycle in a small vacuum chamber. After argon purge, 4 mL dichloroethane was added and the reaction mixture was heated at 120° C. for 10 min, 300W in a Discover SP microwave oven (CEM, Matthews, USA). The solvent and reagents were removed by filtration. The resin was washed with the washing protocol. The reaction progress was monitored by LC-MS. If the ratio between cyclic and linear peptide was less than 6, the metathesis step was repeated until the cyclic peptide was enriched up to a desirable amount (ratio cyclic/linear peptide >6). The resin was used in the next step (cleavage for final product or adding Pyr-Arg) after being washed using the washing protocol.

Macrolactamization. Prior to lactamization, Lys(Alloc) or Dap(Alloc) was introduced to the Pro3 position and Fmoc-Asp(OAll)—OH was incorporated to the His7 position. On the dried resin (0.1 mmol peptide), the Allyl and Alloc protecting groups were removed by treating the resin with Pd(PPh₃)₄(0.25 equiv., 29 mg, 0.025 mmol) and phenylsilane (25 equiv, 311 μL, 2.5 mmol) in DCM under argon atmosphere for 30 min. The resin was washed with 5 mL DCM and 5 mL DMF, 5 min each. Macrocyclization was carried out using DEPBT (5 equiv., 150 mg, 0.5 mmol) and DIPEA (5 equiv., 0.5 mmol, 87 μL). The coupling reagent and DIPEA were dissolved in 5 mL DMF before transferring to the resin and the cyclization reaction was run overnight. The resin was washed with the washing protocol before proceeding to the next step (Fmoc deprotection/peptide elongation).

Modifications post-cyclization. Macrocycles 7 and 8 were synthesized from macrocycle 5 on the resin. After the metathesis step and addition of the Pyr(Boc)-Arg(Pbf)- at the N-terminus to provide 5 (protecting groups were still on), macrocycle 7 was obtained by deprotection of the o-Nosyl group using a mixture of mercapthoethanol (8 equiv., 57 μL, 0.8 mmol), DBU (5 equiv., 0.5 mmol, 76 μL) for 15 min (repeated one more time to ensure full deprotection). The resin was washed with the washing protocol before final cleavage. Macrocycle 8 was synthesized from 7 (with protecting groups on) on the resin by reductive amination using formaldehyde 37% in water (40 equiv., 324 μL, 4 mmol), NaBH(OAc)₃ (20 equiv, 423 mg, 2 mmol) in a mixture of THF-TMOF (1:1) during overnight. The excess NaBH(OAc)₃ was quenched with 3 mL MeOH. After gas evolution ceased, the resin was washed with MeOH, followed by the washing protocol. It should be noted that a portion of the peptide was cleaved during reductive amination, which reduced the yield.

Final cleavage and purification. The final cleavage from resin and simultaneous protecting groups removal were done using a cocktail of trifluoroacetic acid (TFA)/triisopropylsilane (TIPS)/water (95:2.5:2.5). The cleavage reaction was run for 2 h (if the peptide had 0-1 arginine) or 4 h (if peptide had 2 arginines). The mixture was filtered through a glass wool plug to remove solid particles and the solution was dropped slowly into 30 mL methyl tert-butyl ether (pre-cooled at 0° C.) to precipitate the product. The crude peptide was isolated by centrifugation (3000 rpm, 10 min), resuspended in 1 mL of acetic acid (AcOH) 10% and let stand for 10 min. Two layers were separated: residual ether layer (top) vs aqueous layer (bottom). The aqueous layer was isolated and 1 mL AcOH 10% was added to extract the residual peptide from the ether layer. This workup helped to further clean up the mixture and ease purification. The aqueous extracts were combined and filtered before purification. Macrocyclic peptides were purified on HPLC-MS system from Waters (Milford, USA) (column XSELECT™ CSH™ Prep C18 (19×100 mm) packed with 5 μm particles, UV detector 2998, MS SQ Detector 2, Sample manager 2767 and a binary gradient module) using a binary solvent system (acetonitrile/water+0.1% formic acid). Pure fragments (confirmed by UPLC-MS) were combined and lyophilized to give a white solid. The purity of peptides was evaluated using UPLC/MS system from Waters (Milford, USA), using an Acquity UPLC® CSH™ C18 column (2.1×50 mm) packed with 1.7 μm particles with the following gradient: acetonitrile and water with 0.1% HCOOH (0→0.2 min: 5% acetonitrile; 0.2→1.5 min: 5%→95%; 1.5→1.8 min: 95%; 1.8→2.0 min: 95%→5%; 2.0→2.5 min: 5%). All peptides had purity >95%, except 21 (91%), 24 (93%), 32 (93%) and 33 (90%). HRMS spectra were obtained with maXis ESI-Q-Tof instrument from Bruker (Billerica, USA) using electrospray infusion.

### Cell Culture

HEK293 cells stably expressing YFP-tagged human APJ were cultured in DMEM medium supplemented with 10% FBS, at 37° C. under a humid atmosphere maintaining 5% CO₂. Antibiotic G418 (400 μg/mL) was added to maintain a selection pressure for APJ-expressing cells while penicillin/streptomycin (0.1%) were used to prevent bacterial contamination.

### Binding Experiments

Binding experiments were performed on cell membranes of HEK293 stably expressing the YFP-tagged human APJ receptor. Cells were frozen at −80° C. for storage and quickly thawed right before the experiments (1 min at 37° C.). The thawed cells were re-suspended in 5 mL EDTA solution (1 mM EDTA, 50 mM Tris-HCl, pH 7.4), transferred to a 10-mL falcon tube and centrifuged at 3500 g for 15 min at 4° C. to extract cell membranes. The precipitate (cell membranes) was suspended in binding buffer (50 mM Tris-HCl, 0.2% BSA, pH 7.4). Binding assays were run in 96-well plates. Fifteen μg of membrane proteins were incubated with 0.2 nM of radiolabeled [¹²⁵I][Nle75, Tyr77][Pyr¹]-apelin-13 (820 Ci/mmol)³ and test ligand with a range of concentrations from 10⁻¹ to 10⁻¹¹ M in a total volume of 200 μL for 1 h at room temperature.

The incubation mixtures were filtered through a glass fiber filter (Millipore, pre-absorbed of PEI 0.5% for 2 h at 4° C.) to remove unbound ligands, and the filter membranes were washed three times with 170 μL cold binding buffer (4° C.). The γ emission was measured using a 1470 Wizard γ-counter from PerkinElmer (Waltham, USA) (80% efficiency). Non-specific binding did not exceed 5% of total signal (determined by incubation with 10⁻⁵ M of unlabeled Ape13). IC₅₀ values, which represent the concentration of tested ligand displacing 50% of radiolabeled ligand from the receptor, were determined from those results using GraphPad Prism 8. The K_{D} of [Pyr¹]-apelin-13 is 1.8 nM, determined by saturation binding assay. Dissociation constant Kᵢ value was calculated from the IC₅₀ using the Cheng-Prusoff equation and results were displayed as mean±SEM of two to three independent experiments, each done in duplicate (Yung-Chi et al., 1973).

### BRET Assays for Gaᵢ₁ Activation and β-Arrestin2 Recruitment

HEK293 cells were cultivated in high glucose DMEM medium having 10% FBS, 100 U/mL penicillin/streptomycin, 2 mM glutamine, and 20 mM HEPES at 37° C. in T175 flasks under humidified chamber at 5% CO₂. After 24 h, cells were transfected with the plasmids coding for human APJ, Gαᵢ₁-RlucII(91), GFP10-G_{γ2}, and G_{β1} (for BRET-based Gαᵢ₁ activation assay) or coding for APJ-GFP10 and RlucII-β-arrestin2 (for BRET-based β-arrestin2 recruitment assay) using PEI (Murza et al., 2015; Galés et al., 2006; Zimmerman et al., 2012). Before the assays, cells were transferred into white 96-well plates BD Bioscience (Mississauga, Canada) at a concentration of 50,000 cells/well 24 h and incubated at 37° C. overnight. Cells were then washed with phosphate-buffered saline (PBS) and 90 μL Hanks' balanced salt solution was added in each well. Then, cells were stimulated with analogs at concentrations ranging from 10⁻⁵ M to 10⁻¹¹ M for 5 min at 37° C. (Gαᵢ₁) or for 30 min at room temperature (β-arrestin2). After stimulation, 5 μM of coelanterazine 400A was added to each well and the plate was read using the BRET² filter set of a GeniosPro plate reader (Tecan, Austria). The BRET ratio was determined as GFP10ₑₘ/RlucIIₑₘ. Data were plotted and EC₅₀ values were determined using GraphPad Prism 8. Each data point represents the mean±SEM of at least three different experiments each done in triplicate.

### Rat Plasma Stability

Plasma was obtained from male Sprague-Dawley rats by collecting blood in heparin tube and centrifugating at 13,000 rpm to remove blood cells. The isolated plasma was stored at −80° C. and thawed right before the test. In 96-well plate, 6 μL of peptide solution at 1 mM was incubated with 27 μL of plasma at 37° C. in an oven equipped with orbital shaker. Tightly fitted caps were used to seal the wells to avoid water evaporation during incubation. At 0, 1, 2, 4, 6 and 24 h, plasma was inactivated with 140 μL solution ACN-EtOH (1:1) containing 0.25 mM N,N-dimethylbenzamide (internal standard) and the well was sealed again with the tight fitted cap. At 24 h, when all the plasma was inactivated, the caps were removed and the mixtures were transferred into a 96-well filtered plate Impact™ Protein Precipitation (Phenomenex, California, US). A 96-well UPLC plate was put at the bottom to collect the samples. Both plates were centrifuged at 500 g for 10 min at 4° C. to accelerate the filtration. The collected filtrates were diluted with 80 μL water and analyzed in an Acquity UPLC-MS system class H (column Acquity UPLC® protein BEH C4 (2.1×50 mm), 1.7 μm particles with pore 300 Å). The quantity of remaining peptide was plotted into an exponential one-phase decay curve using GraphPad Prism 8 which allowed to calculate peptide half-life. The results were presented as mean±SEM of at least 3 independent experiments, each done in simplicate.

### In Vivo Pharmacokinetics

Male Sprague-Dawley rats of 8-10 weeks were used in this study. Twenty-four hours before the experiments, a jugular vein catheter (Silastic® Laboratory tubing; 0.02 in I.D.×0.037 in O.D.) was surgically inserted for intravenous injections (i.v., 3 mg/kg for analog 42, 43 or Ape13 in saline solution 0.9%, ≈350 μL) and for collecting blood. Animals were placed in a containment chamber prior to i.v. injection to facilitate blood sampling. Blood samples (0.2 mL, corresponding to 0.1 mL plasma after centrifugation) were collected in K2-EDTA microtainer tubes (Sarstedt, Nümbrecht, Germany) at 5, 10, 30, 60, 120 and 240 min (1, 2, 5, 10, 15 min for [Pyr¹]-apelin-13) following i.v. administration. Those samples were immediately stored on crushed ice before being centrifuged at 13000 rpm for 5 min at 4° C. to isolate plasma (upper layer). The resultant plasma was transferred to polypropylene tubes, and immediately frozen at −80° C.

### Sample Preparation

A combination of a protein precipitation and a solid phase extraction step were used to extract the peptides. Plasma sample was defrosted on ice. After vortex agitation (60 s), 100 μL sample was withdraw and 300 μL cold acetonitrile was added to precipitate the plasma proteins. The sample was then vortex (60 s) and centrifuged at 4500 rpm at 4° C. during 10 min. The supernatant was then isolated and directly pass through an HLB prime for additional clean up. The filtrate was diluted 10 times in 0.1% formic acid/water and filtered through a 0.22 μm syringe filter before LC/MS/MS analysis.

### Mass Spectrometry Analysis

Samples were analyzed on a Sciex Qtrap 6500+ equipped with a microflow liquid chromatography (Eksigient M3 microflow) and a UPLC HSS-T3 column (1 mm×100 mm, 1.8 μm, equipped with a 0.2 μm fritted pre-filter). The solvent flow rate was set to 50 μL/min, the column temperature was kept at 40° C. and the injection volume was 3 μL. The mobile phase was 0.1% formic acid/water (A) and 0.1% formic acid/acetonitrile (B). The elution gradient starts with 2% of eluent B, increasing to 95% in 8 min, maintaining at 95% for 2 min and then back to initial conditions in 2 min for a total run time of 13 min. Optimized parameters for peptide fragmentation were obtained by direct infusion of Ape13, 42 and 43 analytical standard solutions at 100 ng/mL. Analysis used two daughter traces (transitions), among them, the most abundant was for quantification and the second most abundant for confirmation.

### In Vivo Blood Pressure Measurement

### Animals

Adult male Sprague-Dawley rats, 8-10 weeks of age (Charles River Laboratories, St-Constant, Quebec, Canada) were kept on a 12 h light/12 h dark cycle with access to food and water ad libitum. The animal experimental protocols were approved by the Animal Care Committee of Université de Sherbrooke and complied with policies and directives of the Canadian Council on Animal Care.

### Blood Pressure Test

Male Sprague-Dawley rats (8-10 weeks of age) were anesthetized with ketamine/xylazine injection (87/13 mg/kg i.m.) and placed in supine position on a thermostatic pad. Their right carotid artery was catheterized with PE 50 (filled with heparinized saline), connected to a Micro-Med transducer (model TDX-300, Calabasas, USA) and Micro-Med blood pressure analyzer (model BPA-100c). Vehicle (isotonic saline) was administered by i.v. bolus, followed 5 min later by the injection of either Ape13, compounds 9, 20, 29, 42 or 43 (given at 19.5 and 65 nmol/kg; volume of 0.25 mL over 10 s) though another catheter (PE10) inserted into the left jugular vein. This i.v. catheter was flushed with saline (0.2 mL) immediately after each injection.

### Echocardiography

Transthoracic echocardiography was performed with a Vevo 3100 ultrasound apparatus using a MX250 transducer (FUJIFILM, VisualSonic, ON, Canada) in Sprague-Dawley rats under isoflurane-anaesthetized (2%; 1.5 mL/min; Baxter), prior (baseline) and 3, 6, and 24 h after subcutaneous injection of peptides (0.2 and 2 μmol/kg). A two-dimensional short axis view of the LV was obtained at the level of the papillary muscle and the M-mode tracing was recorded. From these images, Heart Rate (HR) was calculated and LV End Diastolic (LVEDd) as well as LV End Systolic diameters (LVESd) were measured by the leading-edge method according to the American Society of Echocardiography guidelines. Fractional Shortening (FS) was calculated by the following formula: FS=([LVEDd-LVESd/LVEDd]×100%). Cardiac Output (CO) was assessed from a LV long axis view. Stroke Volume (SV) was calculated according to the Simpson method by tracing the endocardial border in end-systole and end-diastole and CO was obtained as CO=SV×HR.

### Example 2: Synthesis of Apelin 13 Analogs of Table I

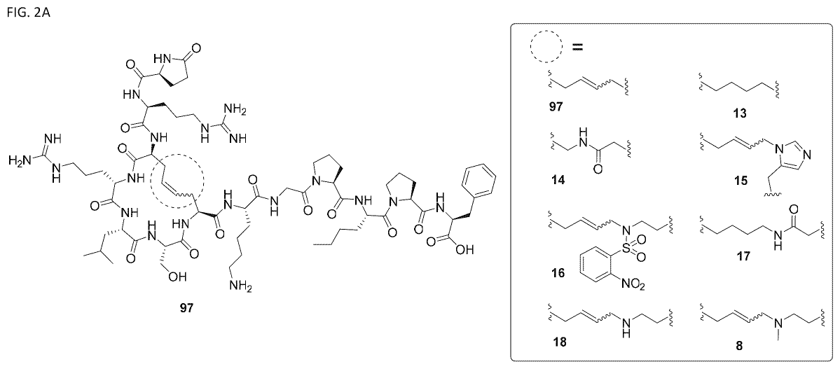

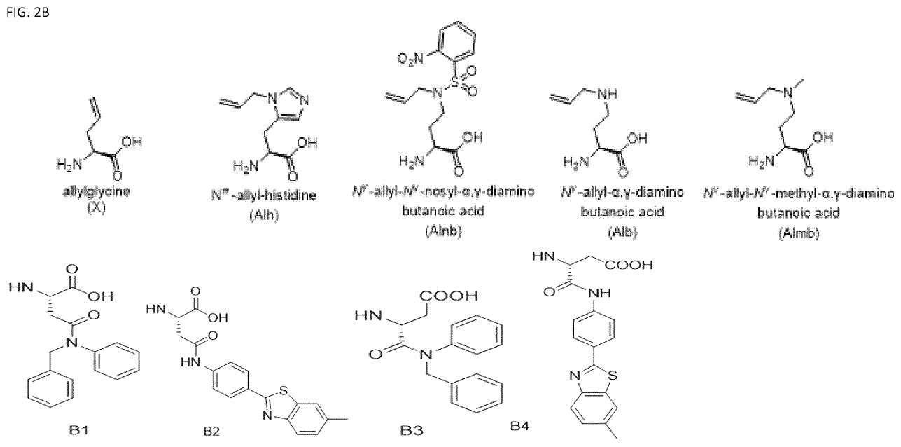

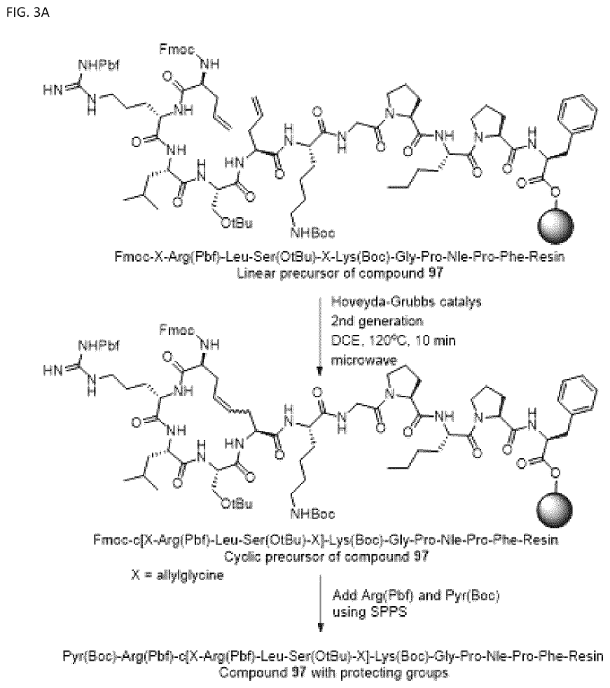

Analogs of 97 were designed and synthesized with various types of macrocyclic linkers, such as saturated hydrocarbon chain (13), lactam group (14, 17), histidine mimetic (15), sulfonamide (16), secondary amine (18) and tertiary amine (19) (FIG. 2A).

Precursor linear peptides were synthesized using classical solid phase peptide synthesis (SPPS) and Fmoc chemistry. In order to build the macrocycles, the Pro3 and His7 residues have been replaced by unnatural amino acids which are part of the linker. For compound 97, allylglycine residues were introduced at both positions to prepare for cyclization using ring closing metathesis (RCM) (FIG. 3A). Compound 13 was obtained from 97 by hydrogenation using 10% Pd/C catalyst (Green et al., 2013). Compounds 14, 17, 46 and 47 were synthesized by macrolactamization according to the protocol described previously (Alcaro et al., 2004) with Dap(Alloc) or Lys(Alloc) in Pro3 position respectively and Asp(OAll) in His7 position. Macrocycles 15, 16, 18, and 19 were also prepared using RCM (FIG. 3B). For this purpose, allylglycine was introduced at the Pro3 position and N^{π}-allyl-histidine (A1h for 15) or NY-allyl-N^{γ}-nosyl-α,γ-diamino-butanoic acid (Alnb for 16, 17, 18) was placed at the His8 position. As described in our previous work (Tran et al., 2018), Pro3→allylglycine mutation was localized between Arg2 and Arg4, which made cyclization of the peptide impossible after the introduction of Arg2, probably due to steric hindrance and catalyst chelation by arginine residues. For this reason, cyclization was carried out before adding the Pyr1-Arg2 moiety (FIG. 3B).

The conversion rate of the RCM step was generally >50%, however, in some cases like compound 14, the yield was around 20-25% at best and longer heating (100° C., 2 h) was required due to the presence of N^{π}-allyl-histidine. The possible explanations are that the imidazole ring of histidine could act as a chelator, poisoning the Hoveyda-Grubbs catalyst.

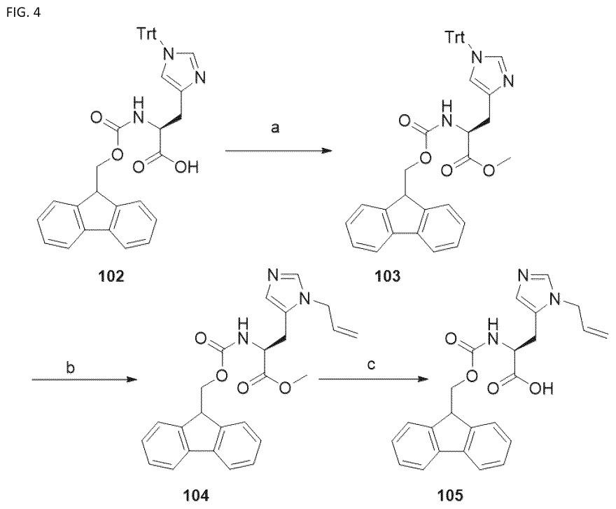

The intermediate Fmoc-L-N^{π}-allyl-histidine-OH (Fmoc-A1h-OH) was prepared in three steps from Fmoc-L-His(Trt)—OH 102 (FIG. 4). The crucial step was to alkylate the N^{π} position of the imidazole ring (103) using allyl triflate generated in situ to provide 104. The sterically hindered trityl (Trt) protecting group remains at the N^{π} position, allowing selective allylation of the N^{α} position. In the next step, the methyl ester 104 was hydrolyzed using HCl 2M in dioxane-water (1:1) under reflux condition to give Fmoc-L-N^{π}-allyl-histidine-OH 105.

Synthesis of analogs 16, 18, 19 requires the residue Fmoc-Alnb-OH (FIG. 3B), which was easily prepared from Fmoc-Dab(Alloc)—OH on the solid phase. The Alloc protecting group was selectively removed using Pd(PPh₃)₄ and PheSiH₃ as scavenger. Nosylation of the γ-amine group was performed with o-nosyl chloride and sym-collidine, followed by allylation using the Fukyama-Mitsunobu reaction to provide the Fmoc-Alnb-containing peptide (FIG. 5).

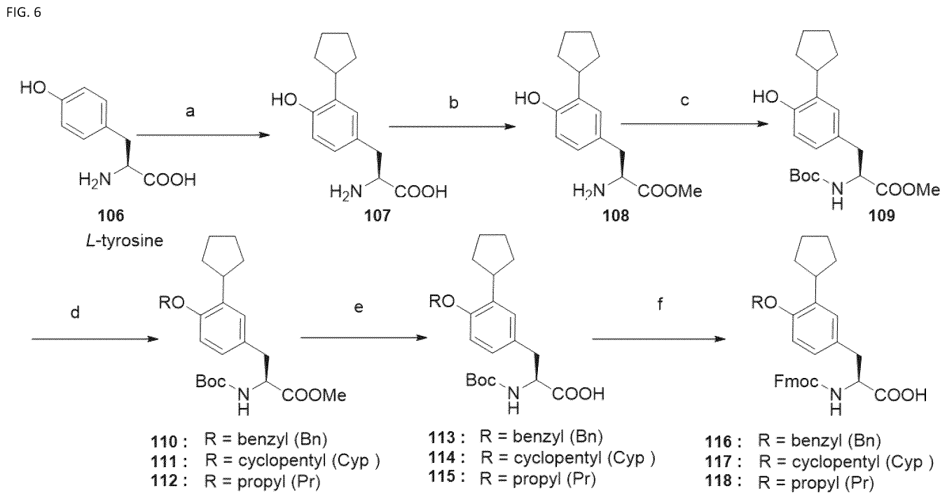

In order to reduce the size of the Ape13 analogs, the N-terminus and C-terminus of the macrocyclic analogs were progressively truncated. The truncated peptides were synthesized using the same protocol as above (FIGS. 3A-B). In the final series, the C-terminal Nle11 residue was substituted by unnatural amino acids. Among the unnatural residues, we have included several Tyr(OBn) analogs, such as cypTyr(OBn), dcypTyr(OBn), cypTyr(OPr), and cypTyr(OCyp) since the incorporation of Tyr(OBn) was previously found to increase the affinity for the binding pocket (Murza et al., 2015). The cyclopentyl group (Cyp) was found to affect the binding and signaling profile of Tyr(OBn) containing peptide in our previous study (Tran et al., 2021). It was introduced on tyrosine (106) using 2 equivalents of cyclopentanol in 85% phosphoric acid under reflux condition to form the precursor 107 (FIG. 6). This intermediate followed a series of transformation (esterification of 107, Boc protection of 108, alkylation of 109, ester hydrolysis of 110-112, Boc removal of 113-115 and Fmoc protection) to provide Fmoc-cypTyr(OBn)—OH (116), Fmoc-cypTyr(OCyp)—OH (117) and Fmoc-cypTyr(OPr)—OH (118), which are suitable for SPPS.

Compounds 20-45, 48-59 were prepared using a method similar to that used for the synthesis of 97. Briefly, the first amino acid was loaded into the Wang resin using Mitsunobu reaction (loading 0.3 mmol/g). The linear peptide was synthesized using Fmoc-based chemistry and the macrocyclization was carried out with Hoveyda-Grubbs catalyst II (120° C., 10 min). The unnatural amino acids bearing terminal alkene such as Lys(N-butenyl),Lys(N-All), Orn(N-butenyl), Dab(N-butenyl) were prepared on resin from Lys(Aloc), Orn(Aloc) and Dab(Aloc) using similar chemistry as the synthesis of Fmoc-Alnb-OH mentioned above.

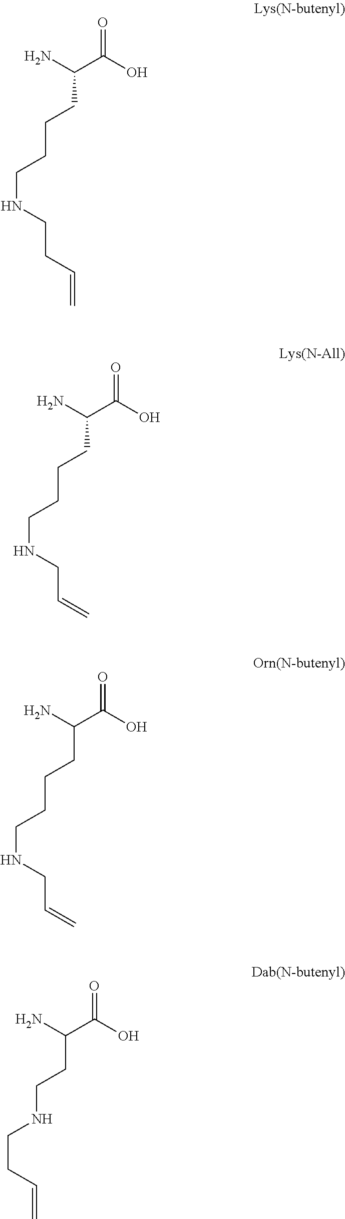

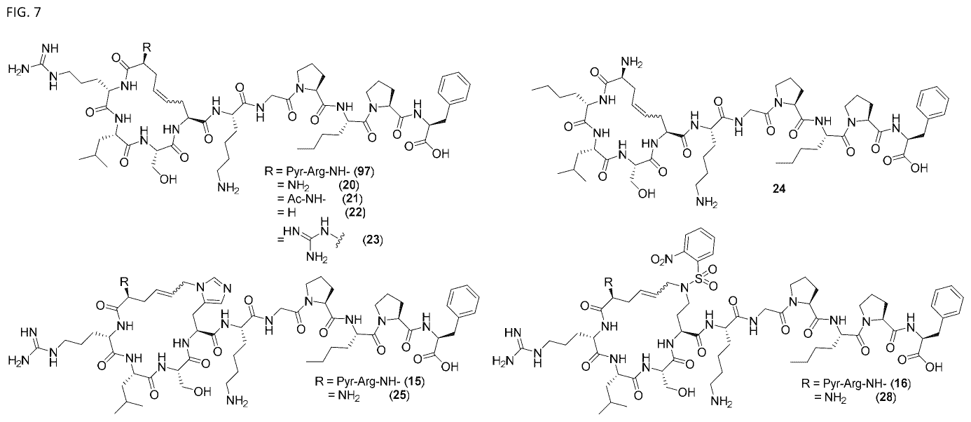

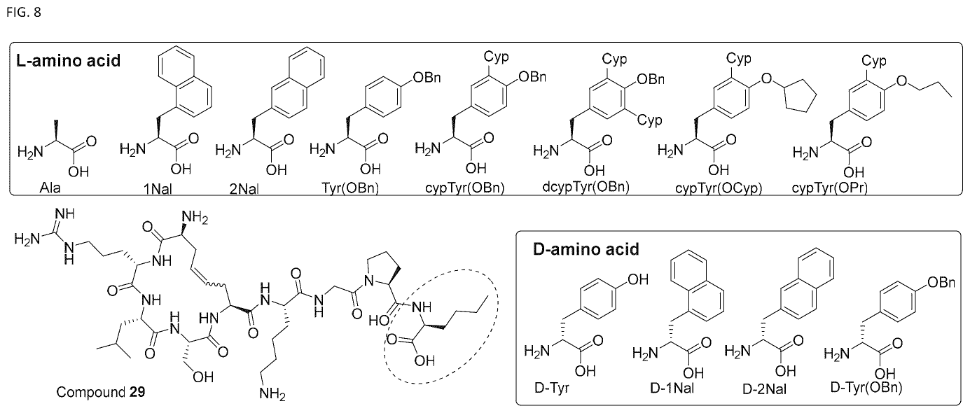

For the modification at the N-terminal end, as in compounds 21, 23, 4, 47, 49, the Fmoc protecting group was removed after the cyclization, the free amino group was derivatized by either acetylation or guanidinylation (using 1H-Pyrazole-1-carboxamidine hydrochloride, CAS: 4023-02-3). See FIGS. 7-8 for compounds 15, 16, 20, 28-29 and 34-45.

### Example 3: Synthesis of Apelin 17 Analog of Table II

Step 1: Loading into resin 2-chlorotrityl 400 mg (loading 0.35 mmol/g).

First, resin was swollen and washed with DCM. Amino acid and DIPEA 2.5 eq. were dissolved in 4 mL DCM and this solution was poured on the resin. The mixture was mixed overnight. Unreacted 2-chlorotrityl chloride was capped using 5 mL of mixture DCM-MeOH-DIPEA (7:2:1). The resin was washed with DMF-DCM-iPrOH-DCM-iPrOH-DCM, 3 min for each solvent after every reaction (capping, Fmoc deprotection, amide coupling).

Step 2: Amino acid Coupling

Fmoc was removed by treating the resin with piperidine 20% in DMF for 10 min. The resin was drained and the deprotection step was repeated one more time. The next amino acid was added by reacting the free N-terminal amine with 5 equiv. of the corresponding Fmoc-protected amino acid, 5 equiv. HATU and 5 equiv. DIPEA. Glu(OAll) and Lys(Alloc) were incorporated to their corresponding position on the peptide sequence.

Step 3: Deprotection of Alloc and Allyl Dry resin was transferred into a 10-mL microwave tube and swelled in 5 mL DCM. The mixture was closed with a cap and bubbled under argon for 10 min before adding PheSiH3.

Tetrakis(triphenylphosphine) palladium (Pd(Ph3)4) was added to the reaction mixture when slightly opening the cap and increasing the argon flow. The mixture was bubbled with argon for 2 min and stirred for 30 min at room temperature. The resin was washed with the washing sequence: DMF-DCM-MeOH-DCM-MeOH-DCM (3 min for 5 mL each solvent).

Step 4: Peptide cyclisation

To a solution of 5 equiv. DEPBT in 4 mL DMF, DIPEA was added. This solution was transferred into the reactor containing the resin and the mixture was shaken overnight.

Ac-Lys(Boc)-Phe-Arg(Pbf)-Arg(Pbf)-Gln(Trt)-Arg(Pbf)-Pro-Arg(Pbf)-Leu-c[Glu-HIs(Trt)-Lys(Boc)-Lys]c-Pro-Nle-Pro-cypTyr(OBn)-Resin (precursor of analog 76).

Step 5: Final cleavage, deprotection and purification

A 5 mL cleavage cocktail of TFA-TIPS-H2O (95:2.5:2.5) was prepared and well mixed. The resin was transferred in a 20-mL vials and the cleavage cocktail was added. This mixture was stirred for 5 h. The resin was filtered out and the filtrate was added dropwise in precooled TBME to precipitate the peptide. The suspension was centrifuged to pull down the solid (3000 rpm×10 min at 4° C.). The supernatant was removed, and residual ether was evaporated under a weak airflow for 30 min. The obtained solid was solubilized in 1900 μL acetic acid 10% in water and filtered through a PTFE 0.22 um filter, into a LC-MS prep vials (3 mL max). The peptide was purified on preparative HPLC-MS using a gradient 10-25% ACN (+0.1% formic) in 15 min. Pure fractions were lyophilized to provide 3 mg of a white powder (analogue 76).

### Example 4: Synthesis of Elabela Analog of Table III

### Materials

Fmoc-protected (L)-amino acids, 2-chlorotrityl chloride resin and [O-(7-azabenzotriazol-1-yl)-1,1,3,3-tetramethyluronium hexafluorophosphate] (HATU) were purchased from Matrix Innovation (Canada). N,N-diisopropylethylamine (DIPEA) and unnatural amino acids were purchased from Chem Impex (USA). Piperidine was purchased from ACP (Canada). All other solvents were purchased from Sigma-Aldrich (Canada) or Fisher Scientific (USA) and were of the highest commercially available purity. All reagents and starting materials were used as received. The peptide elongation was performed with a Symphony™ X peptide synthesizer from Gyros Protein Technology (USA).

### Step 1: Loading of the 2-Chlorotrityl Chloride Resin

To load the first amino acid of the sequence, 2-chlorotrityl chloride resin (0.25 mmol/g, 400 mg) was treated with Fmoc-protected amino acid (1 equiv.), N,N-diisopropylethylamine (DIPEA, 2 equiv.), in dichloromethane (DCM, 4 mL). The mixture was shaken for 2 h on an orbital shaker at room temperature, then the resin was sequentially washed for 3-min periods with DCM (2×5 mL), 2-propanol (1×5 mL), DCM (1×5 mL), 2-propanol (1×5 mL), DCM (2×5 mL). A capping solution of DCM/MeOH/DIPEA (7/2/1, 5 mL) was then added and the mixture shaken for 1 h at room temperature and washed with the above solvent sequence.

### Step 2: Peptide Elongation

The peptide synthesis was carried out with the typical Fmoc solid phase peptide synthesis (SPPS) procedure. 2-chlorotrityl chloride resin (0.25 mmol/g, 400 mg, loaded with the first amino acid of the sequence) was placed in a peptide synthesizer reactor and swell with N,N-dimethylformamide (DMF) (3×6 min, 4.5 mL). To be noted that the resin during coupling, deprotection and washing steps is mixed via N₂ bubbling. The Fmoc group was then deprotected with 20% piperidine/DMF (2×5 min, 4.5 mL), then the subsequent Fmoc-protected amino acid (5 equiv.) was attached in the presence of HATU (5 equiv.), DIPEA (10 equiv.) in DMF/NMP (4.5 mL) and the reaction proceeded for 30 min. Then piperidine (20% in DMF) was used to deprotect the Fmoc group at every step. The resin was washed after each coupling and Fmoc deprotection step with DMF (4 x 1 min 30 s, 4.5 mL).

Step 3: Allyl/Aloc Deptrotection In a typical procedure, after coupling the last amino acid of the sequence, the Allyl/Aloc protecting groups were selectively deprotected with Tetrakis(triphenylphosphine)palladium (Pd(PPh₃)4) (0.2 equiv.) and Phenylsilane (PhSiH₃) (20 equiv.) in Argon degassed DCM (5 mL) and the reaction proceeded during 30 min). The resin was then washed with DMF (3×1 min 30 s, 4.5 mL) and DCM (5×6 min, 4.5 mL).

### Step 4: Macro-Lactamization—Cleavage/Deprotections

Then, the macro-lactamization were carried out with 3-(diethoxyphosphoryloxy)-1,2,3-benzotriazin-4(3H)-one (DEPBT) (5 equiv.) and DIPEA (5 equiv.) in DMF (5 mL) during 16 h. After resin washings (DMF, 5×1 min 30 s, 4.5 mL), macrocycles were cleaved from the resin and the protecting groups were removed with a mixture of TFA (trifluoroacetic acid)/H₂O/TIPS (triisopropylsilane) 95/2.5/2.5, v/v (2 mL/0.2 g of resin) for 4 h at room temperature. The crude was either precipitated in tert-butyl methyl ether (TBME) at 0° C., centrifuged and the supernatant removed, or the crude was directly evaporated under vacuum. The crude was then re-dissolved in 7:3 H₂O/acetonitrile (ACN) and lyophilized before purification by reverse-phase HPLC.

### Step 4: Ring Closing Metathesis (RCM) - Cleavage/Deprotections

Then, the Ring Closing Metathesis (RCM) were carried out in DCE (4 mL), with the Hoveyda Grubbs 2^{nd} generation catalyst (0.2 equiv.) and benzoquinone (1 equiv.) at 50° C. for 1 h in CEM microwave. The resin was then washed with DCM (3 x), MeOH (3 x) and DCM (3 x) and dried before cleavage step. The resin and the protecting groups were removed with a mixture of TFA (trifluoroacetic acid)/H₂O/TIPS (triisopropylsilane) 95/2.5/2.5, v/v (2 mL/0.2 g of resin) for 4 h at room temperature. The crude was either precipitated in tert-butyl methyl ether (TBME) at 0° C., centrifuged and the supernatant removed, or the crude was directly evaporated under vacuum. The crude was then re-dissolved in 7:3 H₂O/acetonitrile (ACN) and lyophilized before purification by reverse-phase HPLC.

### Step 5: Purification and Characterization

The crude was re-suspended in 7:3 H₂O/acetonitrile (ACN) and purified on a preparative HPLC-MS system from Waters (Milford, USA) (column XSELECT™ CSH™ Prep C18 (19×100 mm) packed with 5 μm particles, UV detector 2998, MS SQ Detector 2, Sample manager 2767 and a binary gradient module) using acetonitrile and water+0.1% formic acid as eluents. Pure fractions were lyophilized to give the final product as a white solid. For purity assessment, compounds were analyzed on an UPLC-MS system from Waters (Milford, USA) (column Acquity UPLC® CSH™ C18 (2.1×50 mm) packed with 1.7 μm particles) with the following gradient: acetonitrile and water with 0.1% HCOOH (0→0.2 min: 5% acetonitrile; 0.2→1.5 min: 5%→95%; 1.5→1.8 min: 95%; 1.8→2.0 min: 95%→5%; 2.0→2.5 min: 5%).

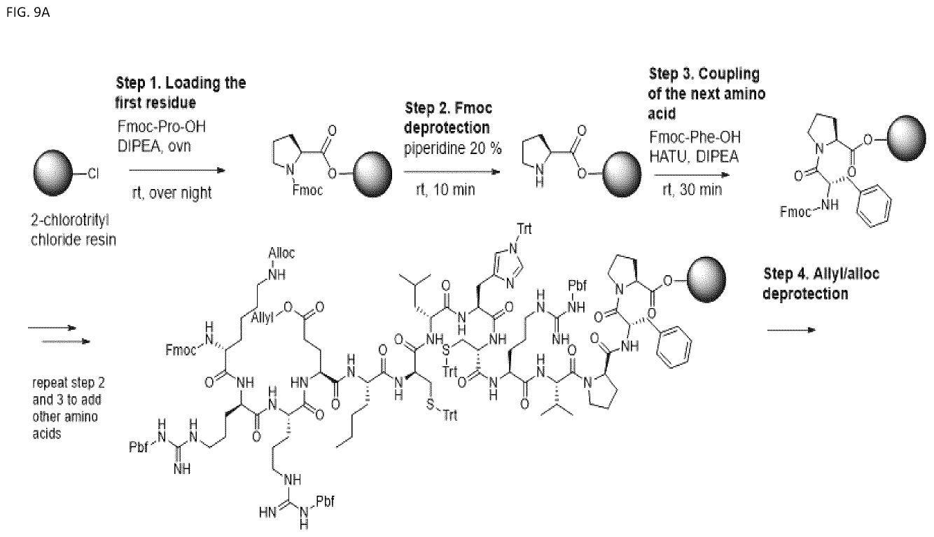

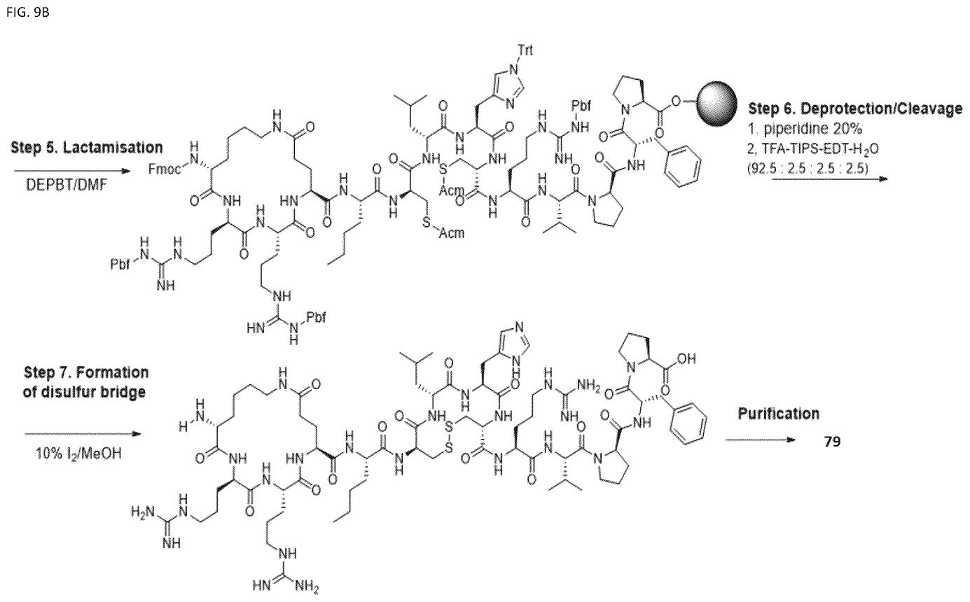

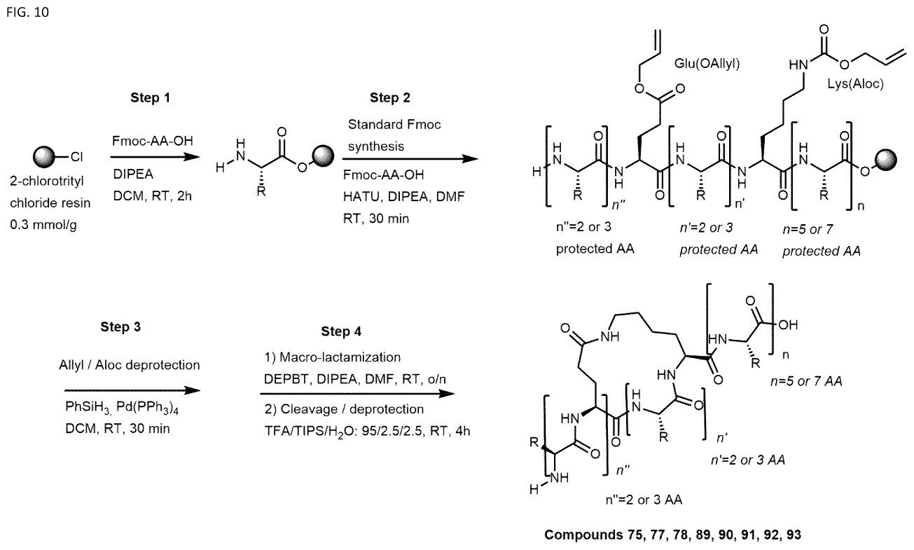

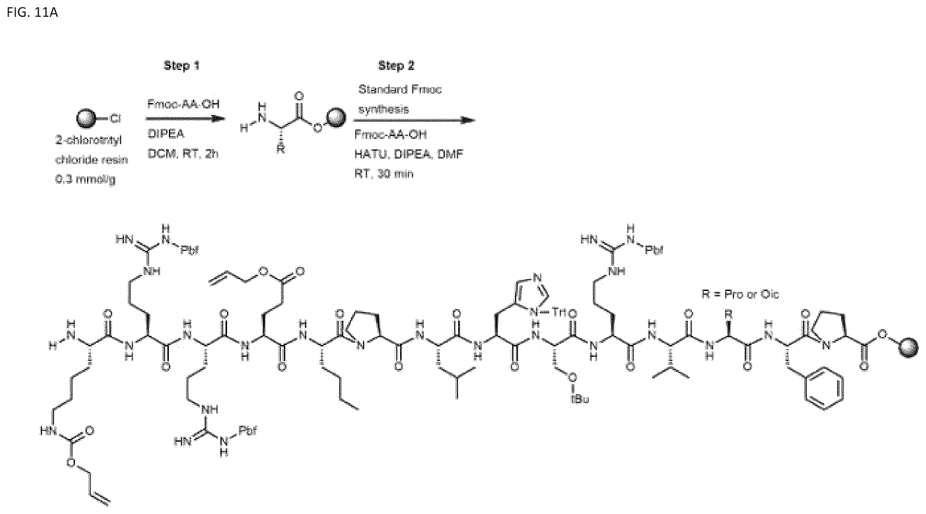

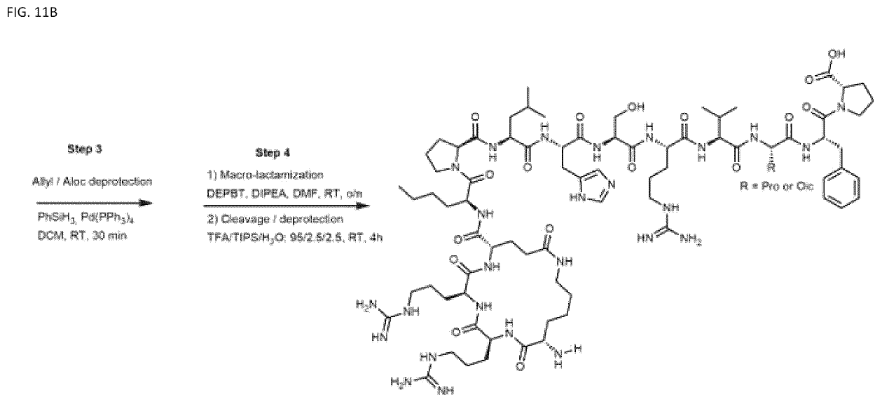

Synthesis schemes for compounds of Table III are also presented in FIGS. 9A-B, 10 and 11A-B.

### Example 5: Characterisation of Apelin 13 Analogues of Table I

| | |
|---|---|
| 3-KT01- | Molecular weight: 1412.619 Da. Chemical formula: |
| 016 | C₆₆H₉₇N₁₉O₁₆. MS (ESI+): 707.62 [M + 2H]²⁺ (calc.: |
| | 706.87). 0.1 mmol scale. Yield: 18.5 mg. Purity: 100%. |
| 4-KT01- | Molecular weight: 1388.637 Da. Chemical formula: |
| 017 | C₆₆H₁₀₁N₁₇O₁₆. MS (ESI+): 695.68 [M + 2H]²⁺ (calc.: |
| | 694.89). 0.1 mmol scale. Yield: 6.1 mg. Purity: 100%. |
| 9-KT02- | Molecular weight: 1477.739 Da. Chemical formula: |
| 98 | C₆₇H₁₀₈N₂₂O₁₆. MS (ESI+): 739.66 [M + 2H]²⁺ (calc.: |
| | 739.43). 0.1 mmol scale. Yield: 4.8 mg. Purity: 100%. |
| 10-KT03- | Molecular weight: 1477.739 Da. Chemical formula: |
| 32 | C₆₇H₁₀₈N₂₂O₁₆. MS (ESI+): 739.65 [M + 2H]²⁺ (calc.: |
| | 739.43). 0.1 mmol scale. Yield: 4.7 mg. Purity: 100%. |
| 11-KT02- | Molecular weight: 1421.6310 Da. Chemical formula: |
| 136 | C₆₃H₁₀₀N₂₂O₁₆. MS (ESI+): 711.47 [M + 2H]²⁺ (calc.: |
| | 711.39). 0.1 mmol scale. Yield: 2 mg. Purity: 92%. |
| 12-KT02- | Molecular weight: 1421.6310 Da. Chemical formula: |
| 137 | C₆₃H₁₀₀N₂₂O₁₆. MS (ESI+): 711.48 [M + 2H]²⁺ (calc.: |
| | 711.39). 0.1 mmol scale. Yield: 2 mg. Purity: 98%. |
| 13-KT01- | Molecular weight: 1449.69 Da. Chemical formula: |
| 125: | C₆₇H₁₀₈N₂₀O₁₆. MS (ESI+): 725.4215 [M + 2H]²⁺ (calc.: |
| | 725.4199). 0.1 mmol scale. Yield: 0.5 mg. Purity: 100%. |
| 14-KT01- | Molecular weight: 1464.67 Da. Chemical formula: |
| 105: | C₆₆H₁₀₅N₂₁O₁₇. MS (ESI+): 732.9084 [M + 2H]²⁺ (calc.: |
| | 732.9071). 0.1 mmol scale. Yield: 3 mg. Purity: 97%. |
| 15-KT01- | Molecular weight: 1527.77 Da. Chemical formula: |
| 98: | C₇₁H₁₁₀N₂₂O₁₆. MS (ESI+): 764.4313 [M + 2H]²⁺ (calc.: |
| | 764.4308). 0.1 mmol scale. Yield: 1.2 mg. Purity: 100%. |
| 16-KT01- | Molecular weight: 1675.9 Da. Chemical formula: |
| 123: | C₇₅H₁₁₄N₂₂O₂₀S. MS (ESI+): 838.4237 [M + 2H]²⁺ (calc.: |
| | 838.4130). 0.1 mmol scale. Yield: 2.1 mg. Purity: 96%. |
| 17-KT01- | Molecular weight: 1506.75 Da. Chemical formula: |
| 106: | C₆₉H₁₁₁N₂₁O₁₇. MS (ESI+): 753.9311 [M + 2H]²⁺ (calc.: |
| | 753.9306). 0.1 mmol scale. Yield: 4 mg. Purity: 100%. |
| 18-KT01- | Molecular weight: 1490.75 Da. Chemical formula: |
| 126: | C₆₉H₁₁₁N₂₁O₁₆. MS (ESI+): 745.9343 [M + 2H]²⁺ (calc.: |
| | 745.9332). 0.1 mmol scale. Yield: 1.8 mg. Purity: 100%. |
| 19-KT01- | Molecular weight: 1504.77 Da. Chemical formula: |
| 122: | C₇₀H₁₁₃N₂₁O₁₆. MS (ESI+): 752.9419 [M + 2H]²⁺ (calc.: |
| | 752.9410). 0.1 mmol scale. Yield: 1.3 mg. Purity: 100%. |
| 20-KT01- | Molecular weight: 1180.4 Da. Chemical formula: |
| 100: | C₅₆H₈₉N₁₅O₁₃. MS (ESI+): 590.8461 [M + 2H]²⁺ (calc.: |
| | 590.8455). 0.1 mmol scale. Yield: 3.4 mg. Purity: 99%. |
| 21-KT01- | Molecular weight: 1222.43 Da. Chemical formula: |
| 118: | C₅₈H₉₁N₁₅O₁₄. MS (ESI+): 611.8519 [M + 2H]²⁺ (calc.: |
| | 611.8508). 0.1 mmol scale. Yield: 1.5 mg. Purity: 91%. |
| 22-KT01- | Molecular weight: 1165.38 Da. Chemical formula: |
| 110: | C₅₆H₈₈N₁₄O₁₃. MS (ESI+): 583.3413 [M + 2H]²⁺ (calc.: |
| | 583.3400). 0.1 mmol scale. Yield: 2 mg. Purity: 95%. |
| 23-KT01- | Molecular weight: 1222.43 Da. Chemical formula: |
| 121: | C₅₇H₉₁N₁₇O₁₃. MS (ESI+): 611.8573 [M + 2H]²⁺ (calc.: |
| | 611.8564). 0.1 mmol scale. Yield: 4.3 mg. Purity: 100%. |
| 24-KT01- | Molecular weight: 1137.37 Da. Chemical formula: |
| 133: | C₅₆H₈₈N₁₂O₁₃. MS (ESI+): 1137.6684 [M + H]⁺ (calc.: |
| | 1137.6667). 0.1 mmol scale. Yield: 0.4 mg. Purity: 93%. |
| 25-KT01- | Molecular weight: 1260.48 Da. Chemical formula: |
| 127: | C₆₀H₉₃N₁₇O₁₃. MS (ESI+): 630.8655 [M + 2H]²⁺ (calc.: |
| | 630.8642). 0.1 mmol scale. Yield: 0.7 mg. Purity: 97%. |
| 26-KT01- | Molecular weight: 1123.3680 Da. Chemical formula: |
| 120 | C₅₄H₈₆N₁₄O₁₂. MS (ESI+): 563.06 [M + 2H]²⁺ (calc.: |
| | 562.33). 0.1 mmol scale. Yield: 0.1 mg. Purity: 98%. |
| 27-KT01- | Molecular weight: 1245.4950 Da. Chemical formula: |
| 111 | C₆₀H₉₂N₁₆O₁₃. MS (ESI+): 624.02 [M + 2H]²⁺ (calc.: |
| | 623.36). 0.1 mmol scale. Yield: 0.2 mg. Purity: 100%. |
| 28-KT01- | Molecular weight: 1408.62 Da. Chemical formula: |
| 135: | C₆₄H₉₇N₁₇O₁₇S. MS (ESI+): 704.8574 [M + 2H]²⁺ (calc.: |
| | 704.8557). 0.1 mmol scale. Yield: 2 mg. Purity: 100%. |
| 29-KT01- | Molecular weight: 936.11 Da. Chemical formula: |
| 116: | C₄₂H₇₃N₁₃O₁₁. MS (ESI+): 958.5439 [M + Na]⁺ (calc.: |
| | 958.5445). 0.1 mmol scale. Yield: 9 mg. Purity: 100%. |
| 30-KT01- | Molecular weight: 668.78 Da. Chemical formula: |
| 115: | C₂₉H₅₂N₁₀O₈. MS (ESI+): 669.4048 [M + H]⁺ (calc.: |
| | 669.4042). 0.1 mmol scale. Yield: 2.4 mg. Purity: 100%. |
| 31-KT03- | Molecular weight: 807.93 Da. Chemical formula: |
| 29: | C₃₆H₆₁N₁₁O₁₀. MS (ESI+): 404.7372 [M + 2H]²⁺ (calc.: |
| | 404.7374). 0.1 mmol scale. Yield: 1.6 mg. Purity: 100%. |
| 32-KT03- | Molecular weight: 850.01 Da. Chemical formula: |
| 50: | C₃₉H₆₇N₁₁O₁₀. MS (ESI+): 425.7615 [M + 2H]²⁺ (calc.: |
| | 425.7609). 0.1 mmol scale. Yield: 2 mg. Purity: 96%. |
| 34-KT03- | Molecular weight: 894.03 Da. Chemical formula: |
| 65: | C₃₉H₆₇N₁₃O₁₁. MS (ESI+): 447.7625 [M + 2H]²⁺ (calc.: |
| | 447.7614). 0.1 mmol scale. Yield: 0.8 mg. Purity: 100%. |
| 35-KT03- | Molecular weight: 1020.18 Da. Chemical formula: |
| 57: | C₄₉H₇₃N₁₃O₁₁. MS (ESI+): 510.7860 [M + 2H]²⁺ (calc.: |
| | 510.7849). 0.1 mmol scale. Yield: 0.5 mg. Purity: 93%. |
| 36-KT03- | Molecular weight: 1020.18 Da. Chemical formula: |
| 58: | C₄₉H₇₃N₁₃O₁₁. MS (ESI+): 510.7859 [M + 2H]²⁺ (calc.: |
| | 510.7849). 0.1 mmol scale. Yield: 0.8 mg. Purity: 90%. |
| 37-KT03- | Molecular weight: 1076.24 Da. Chemical formula: |
| 51: | C₅₂H₇₇N₁₃O₁₂. MS (ESI+): 538.7993 [M + 2H]²⁺ (calc.: |
| | 538.7980). 0.1 mmol scale. Yield: 0.6 mg. Purity: 95%. |
| 38-KT03- | Molecular weight: 1144.36 Da. Chemical formula: |
| 67: | C₅₇H₈₅N₁₃O₁₂. MS (ESI+): 572.8315 [M + 2H]²⁺ (calc.: |
| | 572.8293). 0.1 mmol scale. Yield: 2.6 mg. Purity: 100%. |
| 39-KT03- | Molecular weight: 1212.48 Da. Chemical formula: |
| 68: | C₆₂H₉₃N₁₃O₁₂. MS (ESI+): 606.8608 [M + 2H]²⁺ (calc.: |
| | 606.8606). 0.1 mmol scale. Yield: 0.4 mg. Purity: 100%. |
| 40-KT03- | Molecular weight: 1122.36 Da. Chemical formula: |
| 69: | C₅₅H₈₇N₁₃O₁₂. MS (ESI+): 561.8373 [M + 2H]²⁺ (calc.: |
| | 561.8371). 0.1 mmol scale. Yield: 1.8 mg. Purity: 100%. |
| 41-KT03- | Molecular weight: 1096.32 Da. Chemical formula: |
| 70: | C₅₃H₈₅N₁₃O₁₂. MS (ESI+): 548.8301 [M + 2H]²⁺ (calc.: |
| | 548.8293). 0.1 mmol scale. Yield: 2.1 mg. Purity: 100%. |
| 42-KT04- | Molecular weight: 1020.18 Da. Chemical formula: |
| 43: | C₄₉H₇₃N₁₃O₁₁. MS (ESI+): 510.7867 [M + 2H]²⁺ (calc.: |
| | 510.7849). 0.1 mmol scale. Yield: 6 mg. Purity: 100%. |
| 43-KT04- | Molecular weight: 1020.18 Da. Chemical formula: |
| 44: | C₄₉H₇₃N₁₃O₁₁. MS (ESI+): 510.7865 [M + 2H]²⁺ (calc.: |
| | 510.7849). 0.1 mmol scale. Yield: 9.8 mg. Purity: 100%. |
| 44-KT04- | Molecular weight: 1076.24 Da. Chemical formula: |
| 42F1: | C₅₂H₇₇N₁₃O₁₂. MS (ESI+): 538.8000 [M + 2H]²⁺ (calc.: |
| | 538.7980). 0.1 mmol scale. Yield: 2.7 mg. Purity: 100%. |
| 45-KT04- | Molecular weight: 986.12 Da. Chemical formula: |
| 42b: | C₄₅H₇₁N₁₃O₁₂. MS (ESI+): 493.7762 [M + 2H]²⁺ (calc.: |
| | 493.7745). 0.1 mmol scale. Yield: 2.2 mg. Purity: 100%. |
| 46-KT01- | Molecular weight: 2045.3380 Da. Chemical formula: |
| 145 | C₉₅H₁₄₁N₂₇O₂₄. MS (ESI+): 682.50 [M + 3H]³⁺ (calc.: |
| | 682.70). 0.1 mmol scale. Yield: 1 mg. Purity: 100%. |

### Example 6: Characterization of Apelin 17 Analogues of Table II

| | |
|---|---|
| 62-KT02-62: | Molecular weight: 2158.5540 Da. Chemical formula: |
| | C₉₉H₁₅₆N₃₄O₂₁. MS (ESI+): 541.16 [M + 4H]⁴⁺ (calc.: |
| | 540.56). 0.1 mmol scale. Yield: 15.8 mg. Purity: 99%. |
| 63-KT02-76: | Molecular weight: 2431.9740 Da. Chemical formula: |
| | C₁₁₇H₁₈₃N₃₅O₂₂. MS (ESI+): 609.49 [M + 4H]⁴⁺ (calc.: |
| | 608.87). 0.1 mmol scale. Yield: 3 mg. Purity: 100%. |
| 64-KT02-78: | Molecular weight: 2341.8490 Da. Chemical formula: |
| | C₁₁₀H₁₇₇N₃₅O₂₂. MS (ESI+): 586.72 [M + 4H]⁴⁺ (calc.: |
| | 586.35). 0.1 mmol scale. Yield: 2.4 mg. Purity: 94%. |
| 65-KT02-99: | Molecular weight: 2500.0930 Da. Chemical formula: |
| | C₁₂₂H₁₉₁N₃₅O₂₂. MS (ESI+): 625.84 [M + 4H]⁴⁺ (calc.: |
| | 625.88). 0.1 mmol scale. Yield: 5.1 mg. Purity: 95%. |
| 66-KT03-02: | Molecular weight: 2363.8550 Da. Chemical formula: |
| | C₁₁₂H₁₇₅N₃₅O₂₂. MS (ESI+): 592.46 [M + 4H]⁴⁺ (calc.: |
| | 591.84). 0.1 mmol scale. Yield: 0.9 mg. Purity: 96%. |
| 67-KT02-18: | Molecular weight: 2390.8810 Da. Chemical formula: |
| | C₁₁₃H₁₇₆N₃₆O₂₂. MS (ESI+): 598.61 [M + 4H]⁴⁺ (calc.: |
| | 598.60). 0.1 mmol scale. Yield: 3.7 mg. Purity: 100%. |
| 68-KT02-19: | Molecular weight: 2447.9510 Da. Chemical formula: |
| | C₁₁₄H₁₇₅N₃₇O₂₂S. MS (ESI+): 612.85 [M + 4H]⁴⁺ (calc.: |
| | 612.84). 0.1 mmol scale. Yield: 2.8 mg. Purity: 100%. |
| 69-KT02-20: | Molecular weight: 2390.8810 Da. Chemical formula: |
| | C₁₁₃H₁₇₆N₃₆O₂₂. MS (ESI+): 598.61 [M + 4H]⁴⁺ (calc.: |
| | 598.60). 0.1 mmol scale. Yield: 6.7 mg. Purity: 100%. |
| 70-KT02-21: | Molecular weight: 2447.9510 Da. Chemical formula: |
| | C₁₁₄H₁₇₅N₃₇O₂₂S. MS (ESI+): 612.87 [M + 4H]⁴⁺ (calc.: |
| | 612.84). 0.1 mmol scale. Yield: 3.9 mg. Purity: 100%. |

### Example 7: Characterization of Elabela Analogues of Table III

| | |
|---|---|
| 77-ABB01- | Molecular weight: 1712.00 Da. Chemical formula: |
| 106 | C₇₈H₁₂₂N₂₆O₁₈. MS (ESI+): 429.5 [M + 4H]⁴⁺ (calc.: |
| | 429.0), MS (ESI+): 572.1 [M + 3H]³⁺ (calc.: |
| | 571.7). 0.2 mmol scale. Yield: 8.9 mg. Purity: 100%. |
| 89-KT03- | Molecular weight: 1783.90 Da. Chemical formula: |
| 14 | C₈₀H₁₂₀BrN₂₅O₁₇. MS (ESI+): 595.44 [M + 3H]³⁺ (calc.: |
| | 594.96). 0.2 mmol scale. Yield: 5.0 mg. Purity: 100%. |
| 90-KT03- | Molecular weight: 1726.02 Da. Chemical formula: |
| 16 | C₇₉H₁₂₄N₂₆O₁₈. MS (ESI+): 576.03 [M + 3H]³⁺ (calc.: |
| | 575.99). 0.2 mmol scale. Yield: 36 mg. Purity: 95%. |
| 91-KT03- | Molecular weight: 1726.02 Da. Chemical formula: |
| 17 | C₇₉H₁₂₄N₂₆O₁₈. MS (ESI+): 576.15 [M + 3H]³⁺ (calc.: |
| | 575.99). 0.2 mmol scale. Yield: 20 mg. Purity: 98%. |
| 92-KT03- | Molecular weight: 1790.89 Da. Chemical formula: |
| 15 | C₇₈H₁₂₁BrN₂₆O₁₈. MS (ESI+): 597.78 [M + 3H]³⁺ (calc.: |
| | 597.29). 0.2 mmol scale. Yield: 31 mg. Purity: 100%. |
| 93-KT03- | Molecular weight: 1818.12 Da. Chemical formula: |
| 19 | C₈₅H₁₂₈N₂₆O₁₉. MS (ESI+): 606.86 [M + 3H]³⁺ (calc.: |
| | 606.67). 0.2 mmol scale. Yield: 3.4 mg. Purity: 98%. |
| 74-AM03- | Molecular weight: 1712.00 Da. Chemical formula: |
| 38 | C₇₈H₁₂₂N₂₆O₁₈. MS (ESI+): 428.7 [M + 4H]⁴⁺ (calc.: |
| | 429.0), MS (ESI+): 571.4 [M + 3H]³⁺ (calc.: 571.7) |
| | 0.2 mmol scale. Yield: 9.0 mg. Purity: 96.7%. |
| 75-ABB01- | Molecular weight: 1712.00 Da. Chemical formula: |
| 105 | C₇₈H₁₂₂N₂₆O₁₈. MS (ESI+): 429.5 [M + 4H]⁴⁺ (calc.: |
| | 429.0), MS (ESI+): 572.1 [M + 3H]³⁺ (calc.: |
| | 571.7)0.2 mmol scale. Yield: 7.3 mg. Purity: 100%. |
| 76-AM03- | Molecular weight: 1712.00 Da. Chemical formula: |
| 40 | C₇₈H₁₂₂N₂₆O₁₈. MS (ESI+): 428.7 [M + 4H]⁴⁺ (calc.: |
| | 429.0), MS (ESI+): 571.4 [M + 3H]³⁺ (calc.: 571.7) |
| | 0.2 mmol scale. Yield: 10.7 mg. Purity: 100%. |
| 78-AM03- | Molecular weight: 1766.09 Da. Chemical formula: |
| 67 | C₈₂H₁₂₈N₂₆O₁₈. MS (ESI+): 443.1 [M + 4H]⁴⁺ (calc.: |
| | 442.5), MS (ESI+): 590.3 [M + 3H]³⁺ (calc.: |
| | 589.7) 0.2 mmol scale. Yield: 7.2mg. Purity: 100%. |
| 72-AM03- | Molecular weight: 1714.06 Da. Chemical formula: |
| 37 | C₇₉H₁₂₈N₂₆O₁₇. MS (ESI+): 429.3 [M + 4H]⁴⁺ (calc.: |
| | 429.5), MS (ESI+): 572.1 [M + 3H]³⁺ (calc.: 572.4) |
| | 0.2 mmol scale. Yield: 8.6 mg. Purity: 100%. |
| 73-AM03- | Molecular weight: 1768.15 Da. Chemical formula: |
| 66 | C₈₃H₁₃₄N₂₆O₁₇. MS (ESI+): 443.7 [M + 4H]⁴⁺ (calc.: |
| | 443.0), MS (ESI+): 591.0 [M + 3H]³⁺ (calc.: 590.4) |
| | 0.2 mmol scale. Yield: 5.2 mg. Purity: 100%. |
| 80-AM03- | Molecular weight: 1264.54 Da. Chemical formula: |
| 41: | C₆₁H₉₇N₁₅O₁₄. MS (ESI+): 633.1 [M + 2H]²⁺ (calc.: |
| | 633.3), 422.2 [M + 3H]³⁺ (calc.: 422.5) 0.1 mmol |
| | scale. Yield: 8.9 mg. Purity: 98.2%. |
| 81-ABB01- | Molecular weight: 1264.54 Da. Chemical formula: |
| 108: | C₆₁H₉₇N₁₅O₁₄. MS (ESI+): 633.0 [M + 2H]²⁺ (calc.: |
| | 633.3), 422.3 [M + 3H]³⁺ (calc.: 422.5) 0.1 mmol |
| | scale. Yield: 5.6 mg. Purity: 97.0%. |
| 87-AM03- | Molecular weight: 1217.49 Da. Chemical formula: |
| 42: | C₅₉H₉₂N₁₆O₁₂. MS (ESI+): 609.4 [M + 2H]²⁺ (calc.: |
| | 609.7), 406.6 [M + 3H]³⁺ (calc.: 406.8) 0.1 mmol |
| | scale. Yield: 10.1 mg. Purity: 100%. |
| 88-ABB01- | Molecular weight: 1217.49 Da. Chemical formula: |
| 110: | C₅₉H₉₂N₁₆O₁₂. MS (ESI+): 609.4 [M + 2H]²⁺ (calc.: |
| | 609.7), 406.6 [M + 3H]³⁺ (calc.: 406.8) 0.1 mmol |
| | scale. Yield: 8.9 mg. Purity: 99%. |
| 83-AM03- | Molecular weight: 1189.39 Da. Chemical formula: |
| 43: | C₅₆H₈₄N₁₆O₁₃. MS (ESI+): 595.4 [M + 2H]²⁺ (calc.: |
| | 595.7), 397.2 [M + 3H]³⁺ (calc.: 397.5) 0.1 mmol |
| | scale. Yield: 5.1 mg. Purity: 100%. |
| 84-ABB01- | Molecular weight: 1189.39 Da. Chemical formula: |
| 109: | C₅₆H₈₄N₁₆O₁₃. MS (ESI+): 595.5 [M + 2H]²⁺ (calc.: |
| | 595.7), 397.3 [M + 3H]³⁺ (calc.: 397.5) 0.1 mmol |
| | scale. Yield: 7.6 mg. Purity: 98.6%. |
| 85-AM03- | Molecular weight: 1401.68 Da. Chemical formula: |
| 63: | C₆₇H₁₀₄N₁₈O₁₅. MS (ESI+): 702.3 [M + 2H]²⁺ (calc.: |
| | 701.8), 468.8 [M + 3H]³⁺ (calc.: 468.2) 0.1 |
| | mmol scale. Yield: 4.1 mg. Purity: 100%. |
| 86-AM03- | Molecular weight: 1314.60 Da. Chemical formula: |
| 64: | C₆₄H₉₉N₁₇O₁₃. MS (ESI+): 658.8 [M + 2H]²⁺ (calc.: |
| | 658.3), 439.8 [M + 3H]³⁺ (calc.: 439.2) 0.1 mmol |
| | scale. Yield: 7.3 mg. Purity: 100%. |
| 82-AM03- | Molecular weight: 1217.49 Da. Chemical formula: |
| 65: | C₅₉H₉₂N₁₆O₁₂. MS (ESI+): 610.2 [M + 2H]²⁺ (calc.: |
| | 609.7), 407.4 [M + 3H]³⁺ (calc.: 406.8) 0.1 mmol |
| | scale. Yield: 4.3 mg. Purity: 100%. |
| 79-AM03- | Molecular weight: 1734.1250 Da. Chemical formula: |
| 68: | C₇₇H₁₂₄N₂₆O₁₆S₂. Scale 0.1 mmol - obtained 26.5 mg. |
| | Purity 99%. |
| 94-KT04- | Molecular weight: 1862.7980 Da. Chemical formula: |
| 16 | C₈₀H₁₁₉Br₂N₂₅O₁₇. MS (ESI+): 621.77 [M + 3H]³⁺ (calc.: |
| | 621.58), 466.51 [M + 4H]⁴⁺ (calc.: 466.45). Scale |
| | 0.1 mmol - obtained 4.1 mg. Purity 100%. |

### Example 8: Binding Affinities and Other Biological Properties of the Apelinergic Compounds

Various properties of the apelinergic compounds of the present disclosure were determined and are presented in Tables I-III below.

The dissociation constant, Kᵢ, is reflective of the binding affinity (Kᵢ binding (nM)) of a ligand for its receptor and corresponds to the concentration of ligand that displaced 50% of radiolabeled pyr-apelin-13. It was measured on membranes prepared from HEK293 cells stably expressing human APJ (hAPJ) by a competitive binding assay using [¹²⁵I][Nle⁷⁵, Tyr⁷⁷][Pyr¹]-Ape13.

EC₅₀ Gαi1 measurement determines the concentration of a ligand inducing 50% of the maximal response of Gαᵢ₁ activation by BRET-based biosensors in HEK293 cells expressing the hAPJ receptor.

EC₅₀ β-arr2 measurement determines the concentration of a ligand inducing 50% of the maximal response of b-arrestin2 recruitment by BRET-based biosensors in HEK293 cells expressing the hAPJ receptor.

The half-life in vitro data represent proteolytic stability of analogs after incubation in rat plasma for several time points up to 24 h at 37° C.. The percentage of remaining analogue was calculated by doing the ratio between AU of compound and AUC of internal standard. Half-lives were extrapolated from curves.

TABLE I
Apelin 13 analogues

| Compound No. | | | Kᵢ binding | EC₅₀ Gαᵢ₁ | EC₅₀ β-arr2 | Half-life in |
|---|---|---|---|---|---|---|
| (SEQ ID NO:) | Name | Structure | (nM) | (nM) | (nM) | vitro (h) |
| 60 (47) | Ape 13 | Pyr-R-P-R-L-S-H-K-G-P-M-P-F | 0.6 ± 0.1 | 0.8 ± 0.2 | 37 ± 8 | 0.4 ± 0.1 |
| 95 (82) | | Pyr-R-P-R-L-S-H-K-G-P-Nle-P-F | 0.8 ± 0.2 | | | |
| 97 (84) | | Pyr-R-c[X-R-L-S-X]c-K-G-P-Nle-P-F | 3.0 ± 0.2 | 3.7 ± 0.9 | 126 ± 26 | 4.0 ± 1.3 |
| 1 | KT01-138 | Ac-NH-[B₁-P-Nle-P-X] | >10000 | — | — | |
| 2 | KT01-139 | Ac-NH-[B₂-P-Nle-P-dX] | >10000 | — | — | |
| 3 (1) | KT01-16 | Pyr-c[X-P-R-X]c-S-H-K-G-P-Nle-P-F | 1253 ± 284 | — | — | |
| 4 (2) | KT01-17 | Pyr-c[X-P-R-L-S-X]c-K-G-P-Nle-P-F | 148 ± 21 | — | — | |
| 5 (3) | KT01-30 | Pyr-c[X-P-A-X]c-S-H-K-G-P-Nle-P-F | >10000 | — | — | |
| 6 (4) | KT01-31 | Pyr-c[X-P-A-L-X]c-H-K-G-P-Nle-P-F | >10000 | — | — | |
| 7 (5) | KT01-32 | Pyr-c[X-P-A-L-S-X]c-K-G-P-Nle-P-F | >10000 | — | — | |
| 8 (6) | KT01-33 | Pyr-c[X-P-A-L-S-H-X]c-G-P-Nle-P-F | >10000 | — | — | |
| 9 (7) | KT02-98 | Pyr-R-P-R-L-S-H-K-[dX-P-Nle-P-X] | 52 ± 15 | — | — | |
| 10 (8) | KT03-32 | Pyr-R-P-R-L-S-H-K-[X-P-Nle-P-dX] | 187 ± 20 | — | — | |
| 11 (9) | KT02-136 | Pyr-R-P-R-L-S-H-K-G-P-[X-P-X] | 2.8 ± 2.1 | — | — | |
| 12 (10) | KT02-137 | Pyr-R-P-R-L-S-H-K-G-P-[dX-P-X] | 7.5 ± 0.6 | — | — | |
| 13 (11) | KT01-125 | Pyr-R-c*[X-R-L-S-X]c-K-G-P-Nle-P-F | 4.3 ± 0.3 | 3.3 ± 1.0 | 199 ± 65 | 4.7 ± 1.2 |
| 14 (12) | KT01-105 | Pyr-R-c[Dap-R-L-S-Asp]c-K-G-P-Nle-P-F | 86 ± 8 | — | — | 0.2 ± 0.1 |
| 15 (13) | KT01-98 | Pyr-R-c[X-R-L-S-Alh]c-K-G-P-Nle-P-F | 0.9 ± 0.1 | 1.2 ± 0.1 | 33 ± 4 | 4.2 ± 1.3 |
| 16 (14) | KT01-123 | Pyr-R-c[X-R-L-S-Alnb]c-K-G-P-Nle-P-F | 0.2 ± 0.1 | 3.6 ± 1.7 | 99 ± 29 | 4.3 ± 0.4 |
| 17 (15) | KT01-106 | Pyr-R-c[Lys-R-L-S-Asp]c-K-G-P-Nle-P-F | 39 ± 2 | — | — | 0.1 ± 0.0 |
| 18 (16) | KT01-126 | Pyr-R-c[X-R-L-S-Alb]c-K-G-P-Nle-P-F | 2.5 ± 0.3 | 4.8 ± 2.3 | 237 ± 49 | 2.0 ± 0.1 |
| 19 (17) | KT01-122 | Pyr-R-c[X-R-L-S-Almb]c-K-G-P-Nle-P-F | 1.7 ± 0.1 | 2.6 ± 0.7 | 157 ± 12 | 2.2 ± 0.7 |
| 20 (18) | KT01-100 | NH2-c[X-R-L-S-X]c-K-G-P-Nle-P-F | 4.6 ± 0.3 | 6.7 ± 1.2 | 143 ± 33 | 3.0 ± 0.2 |
| 21 (19) | KT01-118 | Ac-NH-c[X-R-L-S-X]c-K-G-P-Nle-P-F | 28 ± 9 | — | — | — |
| 22 (20) | KT01-110 | Ø-c[X-R-L-S-X]c-K-G-P-Nle-P-F | 61 ± 28 | — | — | 2.0 ± 0.2 |
| 23 (21) | KT01-121 | Guanidine-c[X-R-L-S-X]c-K-G-P-Nle-P-F | 1.7 ± 0.1 | 1.4 ± 0.3 | 110 ± 10 | 3.8 ± 0.7 |
| 24 (22) | KT01-133 | NH₂-c[X-Nle-L-S-X]c-K-G-P-Nle-P-F | 343 ± 137 | — | — | 3.8 ± 0.5 |
| 25 (23) | KT01-127 | NH₂-c[X-R-L-S-Alh]c-K-G-P-Nle-P-F | 201 ± 113 | — | — | 2.0 ± 0.3 |
| 26 (24) | KT01-120 | NH-c[Rx-R-L-S-AIH]-K-G-P-Nle-P-F | 382 ± 224 | — | — | — |
| 27 (25) | KT01-111 | Ø-c[X-R-L-S-AIH]-K-G-P-Nle-P-F | 380 ± 35 | — | — | — |
| 28 (26) | KT01-135 | NH₂-c[X-R-L-S-Alnb]c-K-G-P-Nle-P-F | 8.6 ± 0.6 | 10 ± 3 | 403 ± 41 | 3.1 ± 0.3 |
| 29 (27) | KT01-116 | NH₂-c[X-R-L-S-X]c-K-G-P-Nle | 14 ± 4 | 5.4 ± 0.7 | 743 ± 108 | 0.9 ± 0.4 |
| 30 (28) | KT01-115 | NH₂-c[X-R-L-S-X]c-K | >10000 | — | — | — |
| 31 (29) | KT03-29 | NH₂-c[X-R-L-S-X]c-G-P-Nle | >10000 | — | — | — |
| 32 (30) | KT03-50 | NH₂-c[X-R-L-S-X]c-5Ava-P-Nle | >10000 | — | — | — |
| 33 (31) | KT03-30 | NH₂-[X-R-L-S-X]-P-Nle | >10000 | — | — | — |
| 34 (32) | KT03-65 | NH₂-c[X-R-L-S-X]c-K-G-P-Ala | >10000 | — | — | 0.8 ± 0.3 |
| 35 (33) | KT03-57 | NH₂-c[X-R-L-S-X]c-K-G-P-1Nal | 109 ± 7 | — | — | 0.3 ± 0.1 |
| 36 (34) | KT03-58 | NH₂-c[X-R-L-S-X]c-K-G-P-2Nal | 143 ± 45 | — | — | 0.4 ± 0.1 |
| 37 (35) | KT03-51 | NH₂-c[X-R-L-S-X]c-K-G-P-TyrOBn | 2044 ± 3379 | — | — | 0.5 ± 0.1 |
| 38 (36) | KT03-67 | NH₂-c[X-R-L-S-X]c-K-G-P-cypTyr(OBn) | 86 ± 21 | — | — | 11.3 ± 1.0 |
| 39 (37) | KT03-68 | NH₂-c[X-R-L-S-X]c-K-G-P-dcypTyr(OBn) | 2670 ± 744 | — | — | — |
| 40 (38) | KT03-69 | NH₂-c[X-R-L-S-X]c-K-G-P-cypTyr(OCyp) | 396 ± 159 | — | — | — |
| 41 (39) | KT03-70 | NH₂-c[X-R-L-S-X]c-K-G-P-cypTyr(OPr) | 33 ± 1 | — | — | — |
| 42 (40) | KT04-43 | NH₂-c[X-R-L-S-X]c-K-G-P-(D-1Nal) | 0.6 ± 0.1 | 0.8 ± 0.1 | 31 ± 7 | 5.8 ± 1.9 |
| 43 (41) | KT04-44 | NH₂-c[X-R-L-S-X]c-K-G-P-(D-2Nal) | 5.7 ± 0.4 | 5.0 ± 0.6 | 232 ± 4 | >24 h |
| 44 (42) | KT04-42F1 | NH₂-c[X-R-L-S-X]c-K-G-P-(D-TyrOBn) | 5.3 ± 1.6 | 5.3 ± 1.2 | 301 ± 38 | 6.6 ± 1.7 |
| 45 (43) | KT04-42b | NH₂-c[X-R-L-S-X]c-K-G-P-(D-Tyr) | 16 ± 7 | 9.3 ± 0.9 | 867 ± 84 | 2.4 ± 0.9 |
| 46 (44) | KT01-145 | Ac-c[E-N-T-N-(8-aminooctanoic)-R-P-R-L-K]- | 32 ± 3 | — | — | |
| | | H-K-G-P-Nle-P-F | | — | — | |
| 47 (45) | KT01-144 | Ac-c[D-T-D-W-(8-aminooctanoic)-R-P-R-L-S- | >5000 | — | — | |
| | | H-Dap]-G-P-Nle-P-F | | — | — | |
| 48 | KT01-158 | NH₂-c[X-O-X]-Oic-Nle | >10000 | — | — | |
| 49 | KT01-160 | Guanidine-c[X-R-Hyp(Oall)]-Nle | >10000 | — | — | |
| 50 | KT01-181 | NH₂-R-c[K(N-butenyl)-P-X]-Tyr(Obn) | 7177 ± 1924 | — | — | |
| 51 | KT01-182 | NH₂-c[X-R-Orn(N-butenyl)]-P-Tyr(Obn) | >10000 | — | — | |
| 52 | KT01-191b | NH₂-c[K(N-All)-P-X]-Tyr(Obn) | >10000 | — | — | |
| 53 | KT01-192b | NH₂-c[Dab(N-butenyl)-P-X]-Tyr(Obn) | >10000 | — | — | |
| 54 | AMS01-05A | NH₂-R-c[K(N-butenyl)-Oic-X]-Tyr(Obn) | 13349 | — | — | |
| 55 | AMS01-15A | NH₂-R-c[K(N-butenyl)-P-X]-(beta-hPhe) | >10000 | — | — | |
| 56 | AMS01-16 | NH₂-R-c[K(N-butenyl)-P-X]-Bpa | >10000 | — | — | |
| 57 | AMS01-17A | NH₂-R-c[K(N-butenyl)-P-X]-(D-Tyr(Obn)) | >10000 | — | — | |
| 58 (46) | AMS01-04 | NH₂-R-c[K-P-E]-Tyr(Obn) | >10000 | — | — | |
| 59 | AM02-88 | NH₂-(CH2)4-NH-c[X-K-G-P-X]-Tyr(Obn) | >10000 | — | — | |

a(X) = allylglycine, (Rx) represents Nα-allyl-arginine, (Sx) represents Nα-allyl-serine, (dX) represents D-allylglycine, (Nle) represents norleucine, (B1) represents Nγ-nosyl-Nγ-allyl-α-amino-butanoic acid, (B2) represents Nγ-allyl-α-amino-butanoic acid, (Nle) = norleucine, (Dap) = diamino propionic acid, (Alh) = N^{π}-allyl-histidine, (Alnb) = N^{γ}-allyl-N^{γ}-nosyl-α, γ-diamino-butanoic acid, (Almb) = N^{γ}-allyl-N^{γ}-methyl-α, γ-diamino-butanoic acid, (Alb) = N^{γ}-allyl-α, γ-diamino-butanoic acid, c[...] represents the macrocycle position, c*[...] represents the macrocycle whose double bond on linker was hydrogenated. Ø = desamino analog

TABLE II
Apelin 17 analogues

| | | | Kᵢ | EC₅₀ | EC₅₀ |
|---|---|---|---|---|---|
| Compound No. | | | Binding | Gαᵢ₁ | ß-arr2 |
| (SEQ ID NO:) | Name | Sequence | (nM) | (nM) | (nM) |
| 60 (47) | Ape 13 | Pyr-R-P-R-L-S-H-K-G-P-M-P-F | 0.6 ± 0.1 | 0.8 ± 0.2 | 37 ± 8 |
| 99 (86) | Ape17 | K-F-R-R-Q-R-P-R-L-S-H-K-G-P-M-P-F | — | — | — |
| 61 (48) | Ac-Ape17- | Ac-K-F-R-R-Q-R-P-R-L-S-H-K-G-P-M-Nle-F | 0.15 ± 0.02 | 2.876 | 34.86 |
| | Met15Nle | | | | |
| | (KT03-12) | | | | |
| 62 (49) | KT02-62 | Ac-K-F-R-R-Q-R-P-R-L-[E-H-A-K]-P-A-P-F | 11 ± 7 | 33.42 | 1255 |
| 63 (50) | KT02-76 | Ac-K-F-R-R-Q-R-P-R-L-[E-H-K-K]-P-Nle-P- | 0.25 ± 0.01 | 5.475 | 63.55 |
| | | cypTyrOBn | | | |
| 64 (51) | KT02-78 | Ac-K-F-R-R-Q-R-P-R-L-[E-H-K-K]-P-Nle-P- | 0.77 ± 0.19 | 19.37 | |
| | | cypY | | | |
| 65 (52) | KT02-99 | Ac-K-F-R-R-Q-R-P-R-L-[E-H-K-K]-P-Nle-P- | 4.16 ± 0.68 | 72.96 | >10000 |
| | | dcypTyrOBn | | | |
| 66 (53) | KT03-02 | Ac-K-F-R-R-Q-R-P-R-L-[E-H-K-K]-P-Nle-P- | 1.6 ± 0.1 | 21.67 | 333.5 |
| | | TyrOBn | | | |
| 67 (54) | KT02-18 | Ac-K-F-R-R-Q-R-P-R-L-[E-H-K-K]-P-Nle-P-B1 | 12 ± 3 | | |
| 68 (55) | KT02-19 | Ac-K-F-R-R-Q-R-P-R-L-[E-H-K-K]-P-Nle-P-B2 | 4 ± 1 | | |
| 69 (56) | KT02-20 | Ac-K-F-R-R-Q-R-P-R-L-[E-H-K-K]-P-Nle-P-B3 | 3 ± 1 | | |
| 70 (57) | KT02-21 | Ac-K-F-R-R-Q-R-P-R-L-[E-H-K-K]-P-Nle-P-B4 | 14 ± 5 | | |

B1, B2, B3, B4: see FIG. 2B

TABLE III
Elabela analogues

| Compound No. | | | Binding Kᵢ | EC50 Gail | EC50 ß-arr2 | Half-life in |
|---|---|---|---|---|---|---|
| (SEQ ID NO:) | Name | Peptide sequence a | (nM) | (nM) | (nM) | vitro (h) |
| 60 (47) | Ape13 | Pyr-R-P-R-L-S-H-K-G-P-M-P-F | 0.6 ± 0.1 | 0.8 ± 0.2 | 37 ± 8 | 0.4 ± 0.1 |
| 96 (83) | ELA32 | PyrRPVNLTMRRKLRKHNCLQRRCMPLHSRVPFP | 0.19 ± 0.02 | 5.3 ± 2.5 | 45 ± 5 | |
| 98 (85) | ELA | Pyr-R-R-C-M-P-L-H-S-R-V-P-F-P | 0.93 ± 0.25 | 8.6 ± 1.2 | 166 ± 58 | |
| | (19-32) | | | | | |
| 71 (58) | PM01-06 | Pyr-R-R-C-Nle-P-L-H-S-R-V-P-F-P | 0.14 ± 0.04 | 9.2 ± 2.7 | 167 ± 86 | |
| 72 (59) | AM03-37 | c[K-R-R-E]-Nle-P-L-H-S-R-V-P-F-P | 0.87 ± 0.65 | 1.7 ± 0.4 | 47 ± 2 | |
| 73 (60) | AM03-66 | c[K-R-R-E]-Nle-P-L-H-S-R-V-Oic-F-P | 11 ± 1 | 14 ± 5 | 401 ± 110 | |
| 74 (61) | AM03-38 | Pyr-R-R-c[K-Nle-P-E]-H-S-R-V-P-F-P | 261 ± 129 | — | — | |
| 75 (62) | ABB01- | Pyr-R-R-c[E-Nle-P-K]-H-S-R-V-P-F-P | 59 ± 29 | 19 ± 3 | 2017 ± 479 | |
| | 105 | | | | | |
| 76 (63) | AM03-40 | Pyr-R-R-S-c[K-P-L-H-E]-R-V-P-F-P | 119 ± 55 | — | — | |
| 77 (64) | ABB01- | Pyr-R-R-S-c[E-P-L-H-K]-R-V-P-F-P | 6.2 ± 2.3 | 6.1 ± 1.6 | 247 ± 62 | |
| | 106 | | | | | |
| 78 (65) | AM03-67 | Pyr-R-R-S-c[E-P-L-H-K]-R-V-Oic-F-P | 173 ± 21 | 24 ± 12 | 1818 ± 255 | |
| 79 (66) | AM03-68 | c[K-R-R-E]-Nle-c[C-L-H-C]-R-V-P-F-P | 0.61 ± 0.02 | 4.0 ± 0.8 | 51 ± 3 | >17 |
| 80 (67) | AM03-41 | c[K-Nle-P-L-E]-S-R-V-P-F-P | >5000 | | | |
| 81 (68) | ABB01- | c[E-Nle-P-L-K]-S-R-V-P-F-P | >10000 | | | |
| | 108 | | | | | |
| 82 (69) | AM03-65 | Nle-c[E-L-H-K]-R-V-P-F-P | >10000 | | | |
| 83 (70) | AM03-43 | Nle-P-c[K-H-S-R-E]-P-F-P | >5000 | | | |
| 84 (71) | ABB01- | Nle-P-c[E-H-S-R-K]-P-F-P | 274 ± 184 | | | |
| | 109 | | | | | |
| 85 (72) | AM03-63 | Nle-P-L-c[E-H-S-R-K]-V-P-F-P | >10000 | | | |
| 86 (73) | AM03-64 | Nle-P-L-c[E-H-R-K]-V-P-F-P | >10000 | | | |
| 87 (74) | AM03-42 | Nle-P-L-H-c[K-R-V-E]-F-P | >10000 | | | |
| 88 (75) | ABB01- | Nle-P-L-H-c[E-R-V-K]-F-P | >10000 | | | |
| | 110 | | | | | |
| 89 (76) | KT03-14 | 4BrBz-R-R-S-[E-P-L-H-K]-R-V-P-F-P | 2.3 ± 0.2 | 3.3 ± 0.6 | 50 ± 4 | |
| 90 (77) | KT03-16 | Pyr-hR-R-S-[E-P-L-H-K]-R-V-P-F-P | 16 ± 1 | 15 ± 4 | 394 ± 47 | |
| 91 (78) | KT03-17 | Pyr-R-hR-S-[E-P-L-H-K]-R-V-P-F-P | 9.4 ± 0.6 | 4.3 ± 1.1 | 173 ± 20 | |
| 92 (79) | KT03-15 | Pyr-R-R-S-[E-P-L-H-K]-R-V-P-4BrF-P | 15 ± 2 | 16 ± 3 | 439 ± 91 | |
| 93 (80) | KT03-19 | Pyr-R-R-S-[E-P-L-H-K]-R-V-P-F-Hyp(OBn) | 14 ± 7 | 47 ± 3 | 2199 ± 132 | |
| 94 (81) | KT04-16 | 4BrBz-R-R-S-[E-P-L-H-K]-R-V-P-4BrF-P | 0.29 ± 0.03 | | | |

4BrF: 4-bromo-phenylalanine; 4BrBz: 4-bromobenzoyl

### Example 9: Apelin 13 Analogs on Receptor Binding on Mutant APJ Receptors

The extracellular surface of the APJ receptor has several negatively charged residues on its N-terminal tail and extracellular loops, such as E20, D23, D92, D94, D172, E174, D184, and E194 (Ma et al., 2017). To investigate their role in receptor binding of macrocyclic analogs, the affinities of compounds 20 (N-terminal acetylation), 22 (absence of N-terminal amine), 24 (Arg4Nle) was determined on APJ E20A and D23A mutant receptors since these mutations were previously demonstrated as potential binding sites of cationic parts of apelin (Table IV). The results showed that the affinities of 20, 22 and 24 are less affected by these mutations (<2.5-fold change) compared to those of Ape13 (decreasing 2.4- to 7.7-fold), suggesting that the E20 and D23 of APJ may not play a significant role in the binding of those macrocyclic analogs but they may be important to confer a higher affinity to compounds with positive charges in the N-terminal part.

TABLE IV
Binding affinities of compounds 20,
22, 24 on APJ receptor mutants

| | Binding (nM) | | | |
|---|---|---|---|---|
| | Kᵢ APJ | IC₅₀ APJ | IC₅₀ APJ | IC₅₀ APJ |
| Compounds | WT | WT | E20A | D23A |
| [Pyr¹]- | 0.6 ± 0.1 | 2.2 ± 0.2 | 5.4 ± 0.2 | 17 ± 1 |
| apelin-13 | | | | |
| 20 | 4.6 ± 0.3 | 15 ± 1 | 18 ± 1 | 38 ± 10 |
| 22 | 61 ± 28 | 198 ± 90 | 97 ± 4 | 164 ± 1 |
| 24 | 343 ± 137 | 1111 ± 445 | 1382 ± 167 | 2347 ± 604 |

Kᵢ indicates binding affinity, calculated using the Cheng-Prusoff equation from experimental IC₅₀ which corresponds to the concentration of ligand that displaces 50% radiolabeled [¹²⁵I][Nle⁷⁵, Tyr⁷⁷][Pyr¹]-Ape13. Values Kᵢ represent the mean ± SEM of 2-3 experiments, each performed in duplicate.

### Example 10: Functional Activities of Apelin 13 Analogs

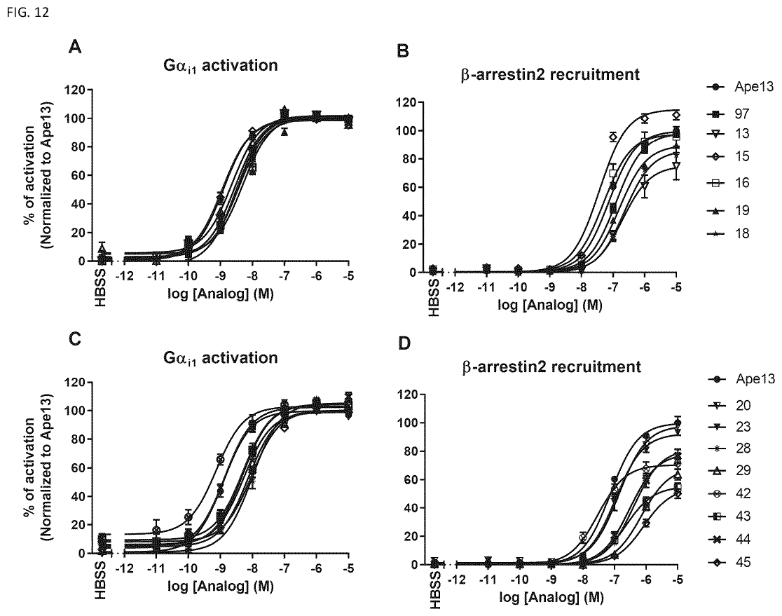

All Apelin 13 analogs with a Kᵢ<20 nM were tested for their ability to activate downstream signaling pathways of the APJ receptor. To this end, BRET-based biosensors were used to monitor G protein- (Gαᵢ₁) activation and β-arrestin2-recruitement. Remarkably, this analysis reveals that some of the macrocycles behave as partial agonists, while others display biased signaling for some of the pathways studied (FIG. 12, panels A-B, Table V).

TABLE V
Affinity, functional activities and plasma stability of Ape13 macrocyclic analogs

| | | | Gαᵢ₁ | ß-arr2 | |
|---|---|---|---|---|---|
| | | Binding | EC₅₀ (nM) | EC₅₀ (nM) | Half-life |
| | Peptide sequence | Kᵢ (nM) | Emax (%) | Emax (%) | t_{1/2} (h) |
| Ape13 | Pyr-R-P-R-L-S-H-K-G-P-M-P-F | 0.6 ± 0.1 | 0.8 ± 0.2 | 37 ± 8 | 0.4 ± 0.1 |
| | | | (100%) | (100%) | |
| 97 | Pyr-R-c[X-R-L-S-X]-K-G-P-Nle-P- | 3.0 ± 0.2 | 3.7 ± 0.9 | 126 ± 26 | 4.0 ± 1.3 |
| | F | | (101%) | (98%) | |
| 13 | Pyr-R-c*[X-R-L-S-X]-K-G-P-Nle-P- | 4.3 ± 0.3 | 3.3 ± 1.0 | 199 ± 65 | 4.7 ± 1.2 |
| | F | | (102%) | (75%) | |
| 15 | Pyr-R-c[X-R-L-S-Alh]-K-G-P-Nle- | 0.9 ± 0.1 | 1.2 ± 0.1 | 33 ± 4 | 4.2 ± 1.3 |
| | P-F | | (99%) | (115%) | |
| 16 | Pyr-R-c[X-R-L-S-Alnb]-K-G-P-Nle- | 0.2 ± 0.1 | 3.6 ± 1.7 | 99 ± 29 | 4.3 ± 0.4 |
| | P-F | | (102%) | (98%) | |
| 18 | Pyr-R-c[X-R-L-S-Alb]-K-G-P-Nle- | 2.5 ± 0.3 | 4.8 ± 2.3 | 237 ± 49 | 2.0 ± 0.1 |
| | P-F | | (100%) | (86%) | |
| 19 | Pyr-R-c[X-R-L-S-Almb]-K-G-P- | 1.7 ± 0.1 | 2.6 ± 0.7 | 157 ± 12 | 2.2 ± 0.7 |
| | Nle-P-F | | (102%) | (90%) | |
| 20 | H₂N-c[X-R-L-S-X]-K-G-P-Nle-P-F | 4.6 ± 0.3 | 6.7 ± 1.2 | 143 ± 33 | 3.0 ± 0.2 |
| | | | (100%) | (98%) | |
| 23 | Guanidine-c[X-R-L-S-X]-K-G-P- | 1.7 ± 0.1 | 1.4 ± 0.3 | 110 ± 10 | 3.8 ± 0.7 |
| | Nle-P-F | | (103%) | (92%) | |
| 28 | H₂N-c[X-R-L-S-Alnb]-K-G-P-Nle- | 8.6 ± 0.6 | 10 ± 3 | 403 ± 41 | 3.1 ± 0.3 |
| | P-F | | (106%) | (82%) | |
| 29 | H₂N-c[X-R-L-S-X]-K-G-P-Nle | 14 ± 4 | 5.4 ± 0.7 | 743 ± 108 | 0.9 ± 0.4 |
| | | | (99%) | (69%) | |
| 42 | H₂N-c[X-R-L-S-X]-K-G-P-(D-1Nal) | 0.6 ± 0.1 | 0.8 ± 0.1 | 31 ± 7 | 5.8 ± 1.9 |
| | | | (103%) | (70%) | |
| 43 | H₂N-c[X-R-L-S-X]-K-G-P-(D-2Nal) | 5.7 ± 0.4 | 5.0 ± 0.6 | 232 ± 4 | >24 |
| | | | (105%) | (55%) | |
| 44 | H₂N-c[X-R-L-S-X]-K-G-P-(D- | 5.3 ± 1.6 | 5.3 ± 1.2 | 301 ± 38 | 6.6 ± 1.7 |
| | TyrOBn) | | (104%) | (78%) | |
| 45 | H₂N-c[X-R-L-S-X]-K-G-P-(D-Tyr) | 1 ± 7 | 9.3 ± 0.9 | 867 ± 84 | 2.4 ± 0.9 |
| | | | (100%) | (54%) | |

### Example 11: In Vitro Plasma Stability

The plasma stability of compounds having an affinity less than 20 nM for the APJ receptor was assessed (Table V above). The first generation of macrocyclic analogs 97, 13, 15, 16, 18 and 19 showed good stability in rat plasma, displaying t_{1/2} ranging from 2 to 5 h, well above Ape13 (t_{1/2} 0.4 h). Peptides with polar groups on the linker (18, 19 t_{1/2}2.0-2.2 h) exhibited lower half-lives (vs 97, 13, 15, 16 t_{1/2}4.0-4.7 h), which suggests that the linker influences the stability of macrocycles.

Truncation of the N-terminal tail (Pyr1-Arg2) slightly reduced plasma stability compared to compound 97, which may be due to exposure of the N-terminal amine, making compounds more vulnerable to aminopeptidases. Nonetheless, analogs 20, 23 and 28 still showed half-lives over 3 h. Macrocyclic analogs were always more stable than their linear analogs. Somewhat surprising is the impact of C-terminal truncation (Pro12-Phe13 removal), which also reduced the peptide stability. Indeed, analog 29 (t_{1/2}0.9 h) having truncated at both C-terminal and N-terminal ends was 3 times less stable than 20, which was truncated only on the N-terminus, and 5 times less stable than full-length analog 13 (t_{1/2}4.7 h). It is known that the C-terminal of Ape13 is cleaved by metalloproteases such as ACE2 and PRCP at the penultimate position (Yang et al., 2017). However, this cleavage site was removed in these truncated analogs.

The peptide stability of analog 29 was improved by the introduction of D-amino acids at the C-terminal Nle11 position. D-amino acids are generally not used by the body and proteases are not evolved for their recognition (Feng et al, 2016) explaining why macrocycles 42, 43, 44, 45 bearing respectively D-1Nal, D-2Nal, D-Tyr(OBn) and D-Tyr substitutions were much more stable than the parent compound 29, with half-lives ranging from 2.4 to >24 h. 43 is the most stable compound of this series, showing a half-life >24 h.

### Example 12: In Vivo Pharmacokinetics

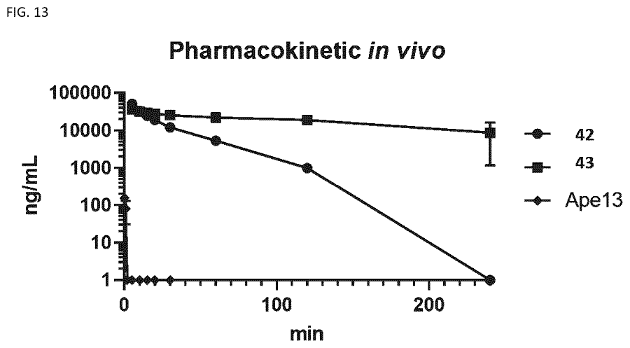

The most potent truncated macrocycle (42) and the most stable analog (43) were selected for in vivo pharmacokinetic profiling. Compounds were administered intravenously to rats via the jugular vein at 3 mg/kg and blood was drawn at 5-, 10-, 15-, 20-, 30-, 60-, 120-, and 240-min post-injection followed by LC/MS-MS analysis (FIG. 13). As expected, 42 and 43 are stable and were detected in rat plasma up to 2 h post-injection while Ape13 completely disappeared after 5 min. Compound 42 displayed a half-life of 24 min and a plasma clearance of 2.29 mL/min/kg (Table VII). In particular, analog 43 had an in vivo t1/2 of 220 min, resulting in a circulating concentration of 8.6 μg/mL at 4 h after injection (compared to 36.1 μg/mL at 5 min). With a long half-life and low plasma clearance (0.34 mL/min/kg), this compound sheds light on the possibility of using Ape13 analogs as cardioprotective drugs with a single bolus injection.

TABLE VII
Pharmacokinetic profile of macrocycles 42 and 43.

| | AUC | | Clearance |
|---|---|---|---|
| Compound | min*(mg/mL) | Half-life (min) | (mL/(min*kg)) |
| Ape13 | 0.0002 ± 0.0001 | <1 min | >100 |
| 42 | 1.33 ± 0.18 | 24 ± 1 | 2.29 ± 0.33 |
| 43 | 8.80 ± 0.41 | 220 ± 1 | 0.34 ± 0.02 |

### Example 13: Effects on Blood Pressure

The inventors assessed the ability of compounds 15, 20, 29, 42, and 43 in rat plasma to modulate blood pressure. Their effect on blood pressure was assessed at two doses of 19.6 and 65 nmol/kg. The dose at 19.6 nmol/kg corresponds to the maximum effect of Ape13 while the higher dose of 65 nmol/kg was chosen to ascertain if a less potent analog can produce an effect.

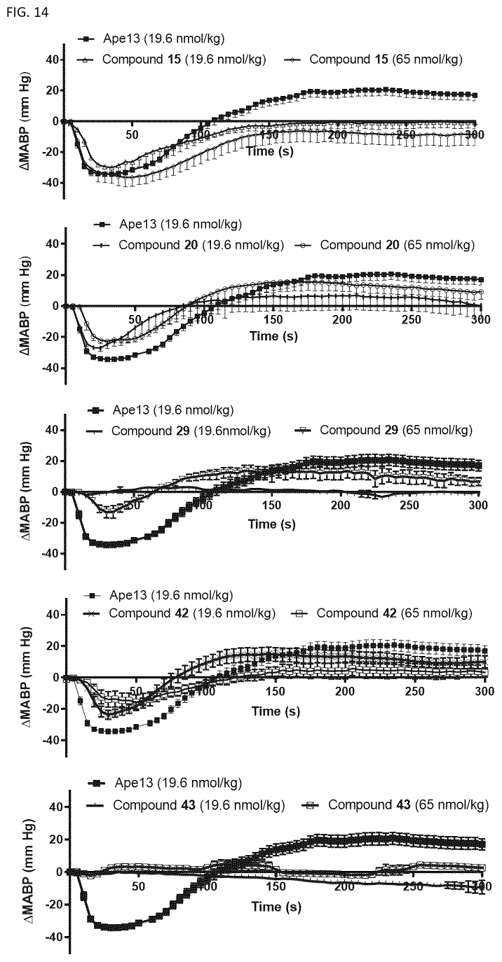

The truncated analogs gradually lost their effect on blood pressure when their size was reduced, along with their ability to recruit β-arrestin2 (Besserer-Offroy et al., 2018). Compound 15 (β-arr2 EC₅₀ 33 nM, Emax 115%) produced a drop in blood pressure (ΔMABP −40 mmHg) similar to Ape13 and the response lasted slightly longer, possibly due to the longer half-life and higher potency in the recruitment of β-arrestin2 (FIG. 14). In contrast, analog 20 (β-arr2 EC₅₀ 143 nM, Eₘₐₓ 98%) having a truncated N-terminal tail, did not reach the same magnitude of response as Ape13 even at the high dose tested (ΔMABP −27 mmHg) (FIG. 14). Similarly, analog 29 (β-arr2 EC₅₀ 743 nM, Emax 69%) having both N-terminal and C-terminal truncation displayed little effect on blood pressure (ΔMABP −13 mmHg) while 43 (β-arr2 EC₅₀ 232 nM, Emax 55%) showed no effect. Compound 42 induced a smaller drop in blood pressure (ΔMABP −24 mmHg) than Ape13 despite a similar potency on the APJ binding (31, Kᵢ 0.6 nM vs Ape13, Kᵢ 0.6 nM) and the recruitment of β-arrestin2 (42, EC₅₀ 31 nM vs Ape13, EC₅₀ 37 nM). The difference could be explained by the lower maximum efficacy of 42 on β-arrestin2 recruitment (Emaₓ 70%), indicative of its partial agonist activity on this pathway. Likewise, compound 43 had only partial efficacy (Eₘₐₓ 55%) and a lower potency (43, EC₅₀ 232 nM) on the b-arrestin2 pathway, which most-likely explains its lack of efficacy on blood pressure.

### Example 14: Effects on Cardiac Performance

Using echocardiography, the inventors studied the cardiac effects of the macrocycles 42 and 43 which exhibit small size, good affinity for APJ as well as improved in vitro and in vivo half-lives.

It should also be reminded that both compounds are full agonists with good potency on Gαᵢ₁ while they are both partial agonists on β-arrestin but that 42 is more potent (by 6.2- to 7.5-fold) than 43 on those pathways.

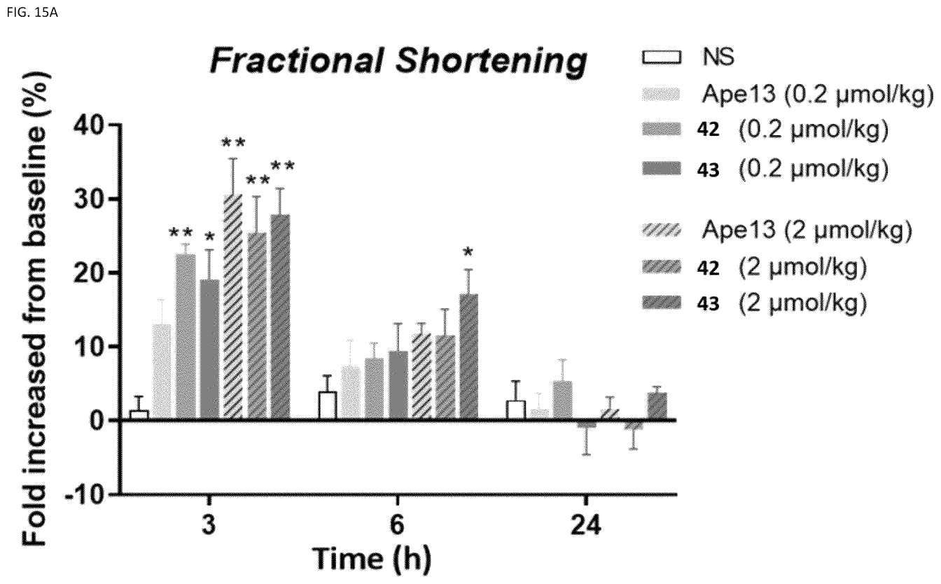

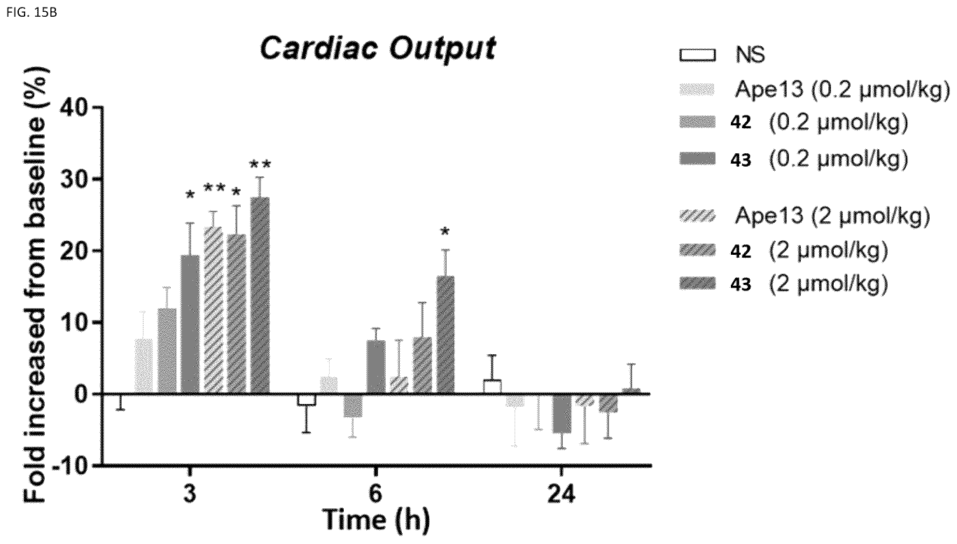

To demonstrate whether peptides with higher stability can maintain the cardiovascular effect following a single bolus administration, Ape13 and the two macrocycles 42 and 43 were administered to rats by subcutaneous injection (s.c.) at two doses, 0.2 μmol/kg (low) and 2 mol/kg (high, nearly 2 mg/kg for the macrocycle). Left ventricular fractional shortening (FS), an indicator of cardiac contraction, as well as cardiac output (CO), which shows overall cardiac performance, were monitored (FIGS. 15A-B). Three hours after injection, no observable effect was detected for Ape13 at 0.2 μmol/kg while 42 showed a significant increase only in FS and 43 showed a significant increase in both FS and CO, consistent with their longer half-life.

At the highest dose tested of 2 μmol/kg, Ape13, 42 and 43 all showed a significant improvement in FS and CO up to 22-27% from baseline 3 h post-injection. Accordingly, a previous study demonstrated that a very high dose of Ape13 (50 mg/kg or approximately 32 μmol/kg, s.c.) can help overcome its short half-life and ensure therapeutically effective concentrations (Onorato et al., 2019). However, most of the compounds tested lost their efficacy at 6 h post-injection due to metabolism and elimination. Only 43 at the highest dose (2 μmol/kg) still showed significant effects on FS (17% increase) and CO (16% increase from baseline) at 6 h. These results are consistent with the pharmacokinetic profile of 42 (t_{1/2} in vivo 24 min) and 43 (t_{1/2} in vivo 220 min). Even at higher doses (3 mg/kg or around 3 μmol/kg), 42 was completely cleared from plasma at 4 h (Table VII), while 43 is expected to drop less than 3 half-lives at 6 h and maintain sufficient concentration to produce an observable effect. Thus, a longer half-live in vivo results in a longer cardiac effect, notably for macrocycle 43.

The scope of the claims should not be limited by the embodiments set forth in the examples, but should be given the broadest interpretation consistent with the description as a whole.

### REFERENCES

Ahn, M.; Murugan, R. N.; Jacob, B.; Hyun, J.-K.; Cheong, C.; Hwang, E.; Park, H.-N.; Seo, J.-H.; Srinivasrao, G.; Lee, K. S.; Shin, S. Y.; Bang, J. K. Discovery of Novel Histidine-Derived Lipo-Amino Acids: Applied in the Synthesis of Ultra-Short Antimicrobial Peptidomimetics Having Potent Antimicrobial Activity, Salt Resistance and Protease Stability. Eur. J. Med. Chem. 2013, 68, 10-18.
Alcaro, M. C.; Sabatino, G.; Uziel, J.; Chelli, M.; Ginanneschi, M.; Rovero, P.; Papini, A. M. On-Resin Head-to-Tail Cyclization of Cyclotetrapeptides: Optimization of Crucial Parameters. J. Pept. Sci. 2004, 10, 218-228.
Angers, S.; Salahpour, A.; Joly, E.; Hilairet, S.; Chelsky, D.; Dennis, M.; Bouvier, M. Detection of B2-Adrenergic Receptor Dimerization in Living Cells Using Bioluminescence Resonance Energy Transfer (BRET). Proc. Natl. Acad. Sci. 2000, 97, 3684-3689.
Besserer-Offroy, É.; Bérubé, P.; Côté, J.; Murza, A.; Longpré, J.-M.; Dumaine, R.; Lesur, O.;
Auger-Messier, M.; Leduc, R.; Marsault, É.; Sarret, P. The Hypotensive Effect of Activated Apelin Receptor Is Correlated with β-Arrestin Recruitment. Pharmacol. Res. 2018, 131, 7-16.
Black, J. W.; Leff, P. Operational Models of Pharmacological Agonism. Proc. R. Soc. Lond. B Biol. Sci. 1983, 220, 141-162.
Chng, S. C.; Ho, L.; Tian, J.; Reversade, B. ELABELA: A Hormone Essential for Heart Development Signals via the Apelin Receptor. Dev. Cell 2013, 27, 672-680.
Galés, C.; Van Durm, J. J. J.; Schaak, S.; Pontier, S.; Percherancier, Y.; Audet, M.; Paris, H.; Bouvier, M. Probing the Activation-Promoted Structural Rearrangements in Preassembled Receptor-G Protein Complexes. Nat. Struct. Mol. Biol. 2006, 13, 778-786.
Green, R.; Peed, J.; Taylor, J. E.; Blackburn, R. A. R.; Bull, S. D. Solid-Phase Asymmetric Synthesis Using a Polymer-Supported Chiral Evans'-Type Oxazolidin-2—One. Nat. Protoc. 2013, 8, 1890-1906.
Gurevich, V. V.; Gurevich, E. V. GPCR Signaling Regulation: The Role of GRKs and Arrestins. Front. Pharmacol. 2019, 10, 125.
Habata, Y.; Fujii, R.; Hosoya, M.; Fukusumi, S.; Kawamata, Y.; Hinuma, S.; Kitada, C.; Nishizawa, N.; Murosaki, S.; Kurokawa, T.; Onda, H.; Tatemoto, K.; Fujino, M. Apelin, the Natural Ligand of the Orphan Receptor APJ, Is Abundantly Secreted in the Colostrum. Biochim. Biophys. Acta BBA—Mol. Cell Res. 1999, 1452, 25-35.
Ma, Y.; Yue, Y.; Ma, Y.; Zhang, Q.; Zhou, Q.; Song, Y.; Shen, Y.; Li, X.; Ma, X.; Li, C.; Hanson, M. A.; Han, G. W.; Sickmier, E. A.; Swaminath, G.; Zhao, S.; Stevens, R. C.; Hu, L. A.; Zhong, W.; Zhang, M.; Xu, F. Structural Basis for Apelin Control of the Human Apelin Receptor. Structure 2017, 25, 858-866.e4.
Macaluso, N. J. M.; Glen, R. C. Exploring the ‘RPRL’ Motif of Apelin-13 through Molecular Simulation and Biological Evaluation of Cyclic Peptide Analogues. Chem Med Chem 2010, 5, 1247-1253.
Maguire, J. J.; Kleinz, M. J.; Pitkin, S. L.; Davenport, A. P. [Pyr1] Apelin-13 Identified as the Predominant Apelin Isoform in the Human Heart Vasoactive Mechanisms and Inotropic Action in Disease. Hypertension 2009, 54, 598-604.
Masri, B.; Morin, N.; Pedebernade, L.; Knibiehler, B.; Audigier, Y. The Apelin Receptor Is Coupled to Gi1 or Gi2 Protein and Is Differentially Desensitized by Apelin Fragments. J. Biol. Chem. 2006, 281, 18317-18326.
Medhurst, A. D.; Jennings, C. A.; Robbins, M. J.; Davis, R. P.; Ellis, C.; Winborn, K. Y.; Lawrie, K. W. M.; Hervieu, G.; Riley, G.; Bolaky, J. E.; Herrity, N. C.; Murdock, P.; Darker, J. G. Pharmacological and Immunohistochemical Characterization of the APJ Receptor and Its Endogenous Ligand Apelin. J. Neurochem. 2003, 84, 1162-1172.
Murza, A.; Besserer-Offroy, É.; Côté, J.; Bérubé, P.; Longpré, J.-M.; Dumaine, R.; Lesur, O.; Auger-Messier, M.; Leduc, R.; Sarret, P.; Marsault, É. C-Terminal Modifications of Apelin-13 Significantly Change Ligand Binding, Receptor Signaling, and Hypotensive Action. J. Med. Chem. 2015, 58, 2431-2440.
Murza, A.; Sainsily, X.; Coquerel, D.; Côté, J.; Marx, P.; Besserer-Offroy, É.; Longpré, J.-M.; Lainé, J.; Reversade, B.; Salvail, D.; Leduc, R.; Dumaine, R.; Lesur, O.; Auger-Messier, M.; Sarret, P.; Marsault, E. Discovery and Structure-Activity Relationship of a Bioactive Fragment of ELABELA That Modulates Vascular and Cardiac Functions. J. Med. Chem. 2016, 59, 2962-2972.
Namkung, Y.; Le Gouill, C.; Lukashova, V.; Kobayashi, H.; Hogue, M.; Khoury, E.; Song, M.; Bouvier, M.; Laporte, S. A. Monitoring G Protein-Coupled Receptor and β-Arrestin Trafficking in Live Cells Using Enhanced Bystander BRET. Nat. Commun. 2016, 7, 12178.
Nyimanu, D.; Kay, R. G.; Sulentic, P.; Kuc, R. E.; Ambery, P.; Jermutus, L.; Reimann, F.; Gribble, F. M.; Cheriyan, J.; Maguire, J. J.; Davenport, A. P. Development and Validation of an LC-MS/MS Method for Detection and Quantification of in Vivo Derived Metabolites of [Pyr1] Apelin-13 in Humans. Sci. Rep. 2019, 9, 19934.
O'Dowd, B. F.; Heiber, M.; Chan, A.; Heng, H. H. Q.; Tsui, L.—C.; Kennedy, J. L.; Shi, X.; Petronis, A.; George, S. R.; Nguyen, T. A Human Gene That Shows Identity with the Gene Encoding the Angiotensin Receptor Is Located on Chromosome 11. Gene 1993, 136, 355-360.
Pauli, A.; Norris, M. L.; Valen, E.; Chew, G.-L.; Gagnon, J. A.; Zimmerman, S.; Mitchell, A.; Ma, J.; Dubrulle, J.; Reyon, D.; Tsai, S. Q.; Joung, J. K.; Saghatelian, A.; Schier, A. F. Toddler: An Embryonic Signal That Promotes Cell Movement via Apelin Receptors. Science 2014, 343, 1248636.
Read, C.; Nyimanu, D.; Williams, T. L.; Huggins, D. J.; Sulentic, P.; Macrae, R. G. C.; Yang, P.; Glen, R. C.; Maguire, J. J.; Davenport, A. P. International Union of Basic and Clinical Pharmacology. CVII. Structure and Pharmacology of the Apelin Receptor with a Recommendation That Elabela/Toddler Is a Second Endogenous Peptide Ligand. Pharmacol. Rev. 2019, 71, 467-502.
Reiter, E.; Ahn, S.; Shukla, A. K.; Lefkowitz, R. J. Molecular Mechanism of β-Arrestin-Biased Agonism at Seven-Transmembrane Receptors. Annu. Rev. Pharmacol. Toxicol. 2012, 52, 179-197.
Tatemoto, K.; Hosoya, M.; Habata, Y.; Fujii, R.; Kakegawa, T.; Zou, M.-X.; Kawamata, Y.; Fukusumi, S.; Hinuma, S.; Kitada, C.; Kurokawa, T.; Onda, H.; Fujino, M. Isolation and Characterization of a Novel Endogenous Peptide Ligand for the Human APJ Receptor. Biochem. Biophys. Res. Commun. 1998, 251, 471-476.
Trân, K.; Murza, A.; Sainsily, X.; Coquerel, D.; Côté, J.; Belleville, K.; Haroune, L.; Longpré, J.-M.; Dumaine, R.; Salvail, D.; Lesur, O.; Auger-Messier, M.; Sarret, P.; Marsault, É. A Systematic Exploration of Macrocyclization in Apelin-13: Impact on Binding, Signaling, Stability, and Cardiovascular Effects. J. Med. Chem. 2018, 61, 2266-2277.
Trân, K.; Van Den Hauwe, R.; Sainsily, X.; Couvineau, P.; Côté, J.; Simard, L.; Echevarria, M.; Murza, A.; Serre, A.; Thdroux, L.; Saibi, S.; Haroune, L.; Longpré, J.-M.; Lesur, O.; Auger-Messier, M.; Spino, C.; Bouvier, M.; Sarret, P.; Ballet, S.; Marsault, É. Constraining the Side Chain of C-Terminal Amino Acids in Apelin-13 Greatly Increases Affinity, Modulates Signaling, and Improves the Pharmacokinetic Profile. J. Med. Chem. 2021, 64, 5345-5364.
Yang, P.; Kuc, R. E.; Brame, A. L.; Dyson, A.; Singer, M.; Glen, R. C.; Cheriyan, J.; Wilkinson, I. B.; Davenport, A. P.; Maguire, J. J. [Pyr¹] Apelin-13(1-12) Is a Biologically Active ACE2 Metabolite of the Endogenous Cardiovascular Peptide [Pyr¹] Apelin-13. Front. Neurosci. 2017, 11, 1-14.
Yung-Chi, C.; Prusoff, W. H. Relationship between the Inhibition Constant (KI) and the Concentration of Inhibitor Which Causes 50 percent Inhibition (I50) of an Enzymatic Reaction. Biochem. Pharmacol. 1973, 22, 3099-3108.
Zhen, E. Y.; Higgs, R. E.; Gutierrez, J. A. Pyroglutamyl Apelin-13 Identified as the Major Apelin Isoform in Human Plasma. Anal. Biochem. 2013, 442, 1-9.
Zimmerman, B.; Beautrait, A.; Aguila, B.; Charles, R.; Escher, E.; Claing, A.; Bouvier, M.; Laporte, S. A. Differential β-Arrestin-Dependent Conformational Signaling and Cellular Responses Revealed by Angiotensin Analogs. Sci. Signal. 2012, 5, ra33-ra33.

1. A compound of formula (II): 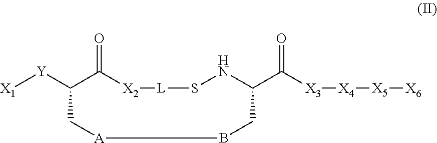
wherein:
X₁ is absent, or is X₇-X₈, wherein
X₇ is —(CH₂)q-CH₃ or —(CF₂)q-CF₃ wherein q is 0 to 11, a natural amino acid, a synthetic amino acid, the side chain of which is H, a —(C1-C12)alkyl, —(CF₂)q-CF₃ wherein q is 0 to 11, —(C3-C8)heteroalkyl, a —(CH₂)p-(C3-C8)aryl, —(CH₂)p-(C3-C8)heteroaryl, —(CH₂)p-(C3-C8)cycloalkyl, or —(CH₂)p-(C3-C8)heterocycloalkyl, wherein p is 0 to 5, wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the alkyl, heteroaryl, aryl, cycloalkyl and heterocycloalkyl is optionally substituted with one or more substituents, wherein each substituent is independently e.g., an halogen, amine, —OH, S, —(C1-C6)alkyl, —O—(C1-C6)alkyl, —(CH₂)p-(C3-C8)aryl, —O—(CH₂)p-(C3-C8)aryl, —(C3-C8)cycloalkyl, or —O—(C3-C8)cycloalkyl, and wherein the heteroatom in the heteroalkyl, heteroaryl or heterocycloalkyl is a N, O or S; and
X₈ is absent, or is a natural or synthetic amino acid, the side chain of which is —CH₂—(CH₂)p-NH2, —CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S;
Y is absent, NH₂—, Ac—NH—, guanidine, or H;
A is —(CH₂)n-; —(CH₂)nNH═C(NH₂)N—CH₂—CH═CH— (preferably allyl-glycine or Nα-allyl-arginine), wherein n is 2, 3 or 4; or —CH═CH—(CH₂)m-, wherein m is 0, 1 or 2;
B is absent or 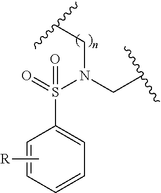
wherein R is O, P, m-alkyl, halogen or nitro and n is 1, 2, or 3; 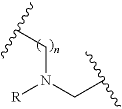
wherein R is H, C3-C7 alkyl, benzyl or arylalkyle and n is 1, 2 or 3; 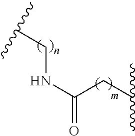
wherein n is 1, 2, 3 or 4 and m is 0 or 1; or 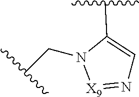
wherein X₉ is CH or N;
X₂ and X₃ are each independently absent, or a natural or synthetic amino acid, the side chain of which is —CH₂—(CH₂)p-NH₂, CH₂—(CH₂)p-guanidine, —(CH₂)p-(C3-C8)cycloalkyl, —(CH₂)p-(C3-C8)heterocycloalkyl, —(CH₂)p-(C3-C8)aryl, or —(CH₂)p-(C3-C8)heteroaryl, wherein p is 0 to 5, wherein the cycloalkyl, heterocycloalkyl, aryl, or heteroaryl is optionally substituted with at least one amino or guanidino group; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S;
X₄ is a natural or non-natural amino acid having a positively charged or uncharged sidechain;
X₅ is Gly, Phe, Leu, Ile, Ser, Aib, Pro, Sar, Oic, βAla, Hyp or Hyp(OBn); and
X₆ is X₁₀-X₁₁-X₁₂, wherein
X₁₀ is any natural amino acid; or a synthetic amino acid, the side chain of which is H, —(CH₂)p-(C3-C8)alkyl, —(CH₂)p-(C3-C8)heteroalkyl, —(CH₂)p-(C3-C8)cycloalkyl, —(CH₂)p-(C3-C8)heterocycloalkyl, —(CH₂)p-(C3-C8)aryl, —(CH₂)p-(C3-C8)heteroaryl, —CH₂—(CH₂)p-NH₂, —CH₂—(CH₂)p-guanidine, wherein p is 0 to 5, wherein the cycloalkyl, heterocycloalkyl, aryl, or heteroaryl is substituted with one or more substituents, wherein each substituent is independently e.g., an halogen, amino group, guanidino group, —OH, S, —(C1-C6)alkyl, —O—(C1-C6)alkyl, —(CH₂)p′—(C3-C8)aryl, —O—(CH₂)p′—(C3-C8)aryl, —(C3-C8)cycloalkyl, or —O—(C3-C8)cycloalkyl, wherein p′ is 0 to 5; wherein the cycloalkyl, heterocycloalkyl, aryl or heteroaryl is optionally fused with one or two (C3-C8)aryl, (C3-C8)heteroaryl, (C3-C8)cycloalkyl or —(C3-C8)heterocycloalkyl; and wherein the heteroatom in the heteroalkyl, heteroaryl or heterocycloalkyl is 1, 2 or 3 N, O or S. In a specific embodiment, X₁₀ is an amino acid, the side chain of which is —(CH₂)p-(C3-C8)alkyl, or —(CH₂)p-(C3-C8)aryl, wherein p is 0 to 5, wherein the aryl is optionally fused with one or two (C3-C8)aryl, and wherein the aryl is optionally substituted with one or more substituents, wherein each substituent is independently O—(C1-C6)alkyl, —(CH₂)p-(C3-C8)aryl, —O—(CH₂)p-(C3-C8)aryl, -(C3-C8)cycloalkyl, or —O—(C3-C8)cycloalkyl, wherein p is 0 to 5. In a specific embodiment, it is not Ala. In a more specific embodiment, X₁₀ is Nle, alpha-methylleucine, cycloleucine, tert-leucine, cyclohexylalanine (e.g., (3-cyclohexyl-L-alanine), alpha-methylphenylalanine, Phe, Tic ((S)—N-Fmoc-tetrahydroisoquinoline-3-carboxylic acid), Tyr, 1Nal, 2Nal, TyrOBn, cypTyr(OBn), dcypTyr(OBn), cypTyr(OCyp), cypTyr(OPr), D-1Nal, D-2Nal, D-TyrOBn, or D-Tyr;
X₁₁ is absent or Gly, Phe, Leu, Ile, Ser, Aib, Pro, Sar, Oic, βAla, Hyp or Hyp(OBn). In a specific embodiment, it is absent or Pro; and
X₁₂ is absent or Phe,
or a stereoisomer or a mixture thereof, or a pharmaceutically acceptable salt, ester or solvate thereof.

2. The compound of claim 1, wherein:
X₂ and X₃ are each independently an amino acid, the side chain of which is —CH₂—(CH₂)p-guanidine, —CH₂—(CH₂)p-NH₂, or —(CH₂)p-imidazole, preferably —CH₂—(CH₂)p-guanidine, or —CH₂—(CH₂)p-NH₂, wherein p is 0 to 4; and/or
X₁₀ is an amino acid, the side chain of which is —(CH₂)p-(C3-C8)alkyl, or —(CH₂)p-(C3-C8)aryl, wherein p is 0 to 5, wherein the aryl is optionally fused with one or two (C3-C8)aryl, and wherein the aryl is optionally substituted with one or more substituents, wherein each substituent is independently —OH, —O—(C1-C6)alkyl, —(CH₂)p′—(C3-C8)aryl, —O—(CH₂)p′—(C3-C8)aryl, —(C3-C8)cycloalkyl, or —O—(C3-C8)cycloalkyl, wherein p′ is 0 to 5,
or a stereoisomer or a mixture thereof, or a pharmaceutically acceptable salt, ester or solvate thereof.

3. The compound of claim 1, wherein:
X₂ and X₃ are each independently Lys, Orn, Dab (2,4-diaminobutyric acid), Dap (2,3-diaminopropionic acid), Arg, hArg, His, Nle, alpha-methylleucine, cycloleucine, tert-leucine, cyclohexylalanine (e.g., (3-cyclohexyl-L-alanine) or alpha-methylphenylalanine;
X₄ is Gly, Phe, Leu, Ile, Ser, Aib, Pro, Sar, Oic, βAla, Hyp or Hyp(OBn);
X₈ is Gly, Phe, Leu, Ile, Ser, Aib, Pro, Sar, Oic, βAla, Hyp or Hyp(OBn); and/or
X₁₀ is X₁₀ is Nle, alpha-methylleucine, cycloleucine, tert-leucine, cyclohexylalanine (e.g., (3-cyclohexyl-L-alanine), alpha-methylphenylalanine, Phe, Tic ((S)—N-Fmoc-tetrahydroisoquinoline-3-carboxylic acid), Tyr, 1Nal, 2Nal, TyrOBn, cypTyr(OBn), dcypTyr(OBn), cypTyr(OCyp), cypTyr(OPr), D-1Nal, D-2Nal, D-TyrOBn, or D-Tyr,
or a stereoisomer or a mixture thereof, or a pharmaceutically acceptable salt, ester or solvate thereof.

4. The compound of claim 3, wherein:
X₂ and X₃ are each independently Lys, Arg, hArg, Nle, Leu, Phe, or Cha;
X₄ is Gly; and/or
X₈ is Pro,
or a stereoisomer or a mixture thereof, or a pharmaceutically acceptable salt, ester or solvate thereof.

5. The compound of any one of claims 1 to 4, wherein:
X₁ is absent;
Y is NH₂, Ac—NH—, guanidine, or H;
A is —CH═CH—(CH₂)m-, wherein m is 0, 1 or 2;
B is absent, 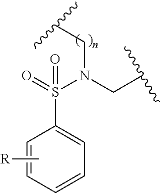
wherein R is O, P, m-alkyl, halogen or nitro and n is 1, 2, or 3, 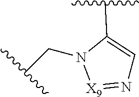
wherein X₉ is CH or N; and/or
X₁₀ is an amino acid, the side chain of which is —(CH₂)p-(C3-C8)alkyl, or —(CH₂)p-(C3-C8)aryl, wherein p is 0 to 5, wherein the aryl is optionally fused with one or two (C3-C8)aryl, and wherein the aryl is optionally substituted with one or more substituents, wherein each substituent is independently —OH, —O—(C1-C6)alkyl, —(CH₂)p′—(C3-C8)aryl, —O—(CH₂)p′—(C3-C8)aryl, —(C3-C8)cycloalkyl, or —O—(C3-C8)cycloalkyl, wherein p′ is 0 to 5,
or a stereoisomer or a mixture thereof, or a pharmaceutically acceptable salt, ester or solvate thereof.

6. The compound of claim 5, wherein X₁₀ is Nle or D-1Nal, or a stereoisomer or a mixture thereof, or a pharmaceutically acceptable salt, ester or solvate thereof.

7. The compound of any one of claims 1 to 4, wherein:
X₁ is X₇-X₈; and/or
Y is absent,
or a stereoisomer or a mixture thereof, or a pharmaceutically acceptable salt, ester or solvate thereof.

8. The compound of claim 7, wherein:
X₁ is X₇-X₈ and X₈ is an amino acid, the side chain of which is —CH₂—(CH₂)p-guanidine, —CH₂—(CH₂)p-NH₂, or —(CH₂)p-imidazole, preferably —CH₂—(CH₂)p-guanidine, or —CH₂—(CH₂)p-NH₂, wherein p is 0 to 4,
or a stereoisomer or a mixture thereof, or a pharmaceutically acceptable salt, ester or solvate thereof.

9. The compound of claim 7 or 8, wherein
A is —(CH₂)n- or —CH═CH—(CH₂)m-, wherein m is 0, 1 or 2,
or a stereoisomer or a mixture thereof, or a pharmaceutically acceptable salt, ester or solvate thereof.

10. A compound of any one of formula (I) to (VIII), or a stereoisomer or a mixture thereof, or a pharmaceutically acceptable salt, ester or solvate thereof.

11. The compound of claim 10, which is any one of compounds 3-4, 9-29, 35-46, 62-70, 72-79, 84, and 89-94, preferably any one of compounds 11, 13, 15-16, 18-20, and 42-44:

| Compound | | |
|---|---|---|
| No. | Name | Structure |
| 3 | KT01-16 | Pyr-c[X-P-R-X]c-S-H-K-G-P-Nle-P-F |
| 4 | KT01-17 | Pyr-c[X-P-R-L-S-X]c-K-G-P-Nle-P-F |
| 9 | KT02-98 | Pyr-R-P-R-L-S-H-K-[dX-P-Nle-P-X] |
| 10 | KT03-32 | Pyr-R-P-R-L-S-H-K-[X-P-Nle-P-dX] |
| 11 | KT02-136 | Pyr-R-P-R-L-S-H-K-G-P-[X-P-X] |
| 12 | KT02-137 | Pyr-R-P-R-L-S-H-K-G-P-[dX-P-X] |
| 13 | KT01-125 | Pyr-R-c*[X-R-L-S-X]c-K-G-P-Nle-P-F |
| 14 | KT01-105 | Pyr-R-c[Dap-R-L-S-Asp]c-K-G-P-Nle-P-F |
| 15 | KT01-98 | Pyr-R-c[X-R-L-S-Alh]c-K-G-P-Nle-P-F |
| 16 | KT01-123 | Pyr-R-c[X-R-L-S-Alnb]c-K-G-P-Nle-P-F |
| 17 | KT01-106 | Pyr-R-c[Lys-R-L-S-Asp]c-K-G-P-Nle-P-F |
| 18 | KT01-126 | Pyr-R-c[X-R-L-S-Alb]c-K-G-P-Nle-P-F |
| 19 | KT01-122 | Pyr-R-c[X-R-L-S-Almb]c-K-G-P-Nle-P-F |
| 20 | KT01-100 | NH₂-c[X-R-L-S-X]c-K-G-P-Nle-P-F |
| 21 | KT01-118 | Ac-NH-c[X-R-L-S-X]c-K-G-P-Nle-P-F |
| 22 | KT01-110 | Ø-c[X-R-L-S-X]c-K-G-P-Nle-P-F |
| 23 | KT01-121 | Guanidine-c[X-R-L-S-X]c-K-G-P-Nle-P-F |
| 24 | KT01-133 | NH₂-c[X-Nle-L-S-X]c-K-G-P-Nle-P-F |
| 25 | KT01-127 | NH₂-c[X-R-L-S-Alh]c-K-G-P-Nle-P-F |
| 26 | KT01-120 | NH-c[Rx-R-L-S-AlH]-K-G-P-Nle-P-F |
| 27 | KT01-111 | Ø-c[X-R-L-S-AlH]-K-G-P-Nle-P-F |
| 28 | KT01-135 | NH₂-c[X-R-L-S-Alnb]c-K-G-P-Nle-P-F |
| 29 | KT01-116 | NH₂-c[X-R-L-S-X]c-K-G-P-Nle |
| 35 | KT03-57 | NH₂-c[X-R-L-S-X]c-K-G-P-1Nal |
| 36 | KT03-58 | NH₂-c[X-R-L-S-X]c-K-G-P-2Nal |
| 37 | KT03-51 | NH₂-c[X-R-L-S-X]c-K-G-P-TyrOBn |
| 38 | KT03-67 | NH₂-c[X-R-L-S-X]c-K-G-P-cypTyr(OBn) |
| 39 | KT03-68 | NH₂-c[X-R-L-S-X]c-K-G-P-dcypTyr(OBn) |
| 40 | KT03-69 | NH₂-c[X-R-L-S-X]c-K-G-P-cypTyr(OCyp) |
| 41 | KT03-70 | NH₂-c[X-R-L-S-X]c-K-G-P-cypTyr(OPr) |
| 42 | KT04-43 | NH₂-c[X-R-L-S-X]c-K-G-P-(D-1Nal) |
| 43 | KT04-44 | NH₂-c[X-R-L-S-X]c-K-G-P-(D-2Nal) |
| 44 | KT04-42F1 | NH₂-c[X-R-L-S-X]c-K-G-P-(D-TyrOBn) |
| 45 | KT04-42b | NH₂-c[X-R-L-S-X]c-K-G-P-(D-Tyr) |
| 46 | KT01-145 | Ac-c[E-N-T-N-(8-aminooctanoic)-R-P-R-L-K]-H-K-G-P-Nle-P-F |
| 62 | KT02-62 | Ac-K-F-R-R-Q-R-P-R-L-[E-H-A-K]-P-A-P-F |
| 63 | KT02-76 | Ac-K-F-R-R-Q-R-P-R-L-[E-H-K-K]-P-Nle-P-cypTyrOBn |
| 64 | KT02-78 | Ac-K-F-R-R-Q-R-P-R-L-[E-H-K-K]-P-Nle-P-cypY |
| 65 | KT02-99 | Ac-K-F-R-R-Q-R-P-R-L-[E-H-K-K]-P-Nle-P-dcypTyrOBn |
| 66 | KT03-02 | Ac-K-F-R-R-Q-R-P-R-L-[E-H-K-K]-P-Nle-P-TyrOBn |
| 67 | KT02-18 | Ac-K-F-R-R-Q-R-P-R-L-[E-H-K-K]-P-Nle-P-B1 |
| 68 | KT02-19 | Ac-K-F-R-R-Q-R-P-R-L-[E-H-K-K]-P-Nle-P-B2 |
| 69 | KT02-20 | Ac-K-F-R-R-Q-R-P-R-L-[E-H-K-K]-P-Nle-P-B3 |
| 70 | KT02-21 | Ac-K-F-R-R-Q-R-P-R-L-[E-H-K-K]-P-Nle-P-B4 |
| 72 | AM03-37 | c[K-R-R-E]-Nle-P-L-H-S-R-V-P-F-P |
| 73 | AM03-66 | c[K-R-R-E]-Nle-P-L-H-S-R-V-Oic-F-P |
| 74 | AM03-38 | Pyr-R-R-c[K-Nle-P-E]-H-S-R-V-P-F-P |
| 75 | ABB01-105 | Pyr-R-R-c[E-Nle-P-K]-H-S-R-V-P-F-P |
| 76 | AM03-40 | Pyr-R-R-S-c[K-P-L-H-E]-R-V-P-F-P |
| 77 | ABB01-106 | Pyr-R-R-S-c[E-P-L-H-K]-R-V-P-F-P |
| 78 | AM03-67 | Pyr-R-R-S-c[E-P-L-H-K]-R-V-Oic-F-P |
| 79 | AM03-68 | c[K-R-R-E]-Nle-c[C-L-H-C]-R-V-P-F-P |
| 84 | ABB01-109 | Nle-P-c[E-H-S-R-K]-P-F-P |
| 89 | KT03-14 | 4BrBz-R-R-S-[E-P-L-H-K]-R-V-P-F-P |
| 90 | KT03-16 | Pyr-hR-R-S-[E-P-L-H-K]-R-V-P-F-P |
| 91 | KT03-17 | Pyr-R-hR-S-[E-P-L-H-K]-R-V-P-F-P |
| 92 | KT03-15 | Pyr-R-R-S-[E-P-L-H-K]-R-V-P-4BrF-P |
| 93 | KT03-19 | Pyr-R-R-S-[E-P-L-H-K]-R-V-P-F-Hyp(OBn) |
| 94 | KT04-16 | 4BrBz-R-R-S-[E-P-L-H-K]-R-V-P-4BrF-P |

wherein X represents allylglycine and dX represents D-allylglycine,
or a stereoisomer or a mixture thereof, or a pharmaceutically acceptable salt, ester or solvate thereof.

12. The compound of claim 11, which is any one of compounds 13-25, 27-29, 35-37 and 42-45, or a stereoisomer or a mixture thereof, or a pharmaceutically acceptable salt, ester or solvate thereof.

13. A pharmaceutical composition comprising the compound, stereoisomer, mixture, pharmaceutically acceptable salt, ester or solvate of any one of claims 1 to 12, and at least one pharmaceutically acceptable carrier or excipient.

14. A method of using a compound of any one of formula (I) to (IV), or a stereoisomer or a mixture thereof, or a pharmaceutically acceptable salt, ester or solvate thereof, for treating a cardiovascular disease in a subject in need thereof, comprising administering an effective amount of the compound to the subject.

15. The method of claim 14, wherein the compound is any one of compounds 3-4, 9-29, and 35-46 as defined in claim 10, or a stereoisomer or a mixture thereof, or a pharmaceutically acceptable salt, ester or solvate thereof.

16. The method of claim 14, wherein the compound is of formula (II) as defined in any one of claims 1 to 8.

17. The method of claim 16, wherein the compound is any one of compounds 13-25, 27-29, 35, 36-37 and 42-45, preferably any one of compounds 13, 15-16, 18-20, 23 and 42-44, as defined in claim 10, or a stereoisomer or a mixture thereof, or a pharmaceutically acceptable salt, ester or solvate thereof.

18. The method of claim 17, wherein the compound is compound 42 or 43, or a stereoisomer or a mixture thereof, or a pharmaceutically acceptable salt, ester or solvate thereof.
